(19) United States
(12) Patent Application Publication
(10) Pub. No.: US 2013/0045975 A1
(43) Pub. Date: Feb. 21, 2013

(54) BICYCLIC HETEROARYL INHIBITORS OF PDE4

(71) Applicants: Steven P. Govek, San Diego, CA (US); Clay Beauregard, Fort Worth, TX (US); Daniel A. Gamache, Arlington, TX (US); Mark R. Hellberg, Arlington, TX (US); Stewart A. Noble, San Diego, CA (US); Andrew K. Shiau, San Diego, CA (US); David J. Thomas, San Diego, CA (US); John M. Yanni, Burleson, TX (US)

(72) Inventors: Steven P. Govek, San Diego, CA (US); Clay Beauregard, Fort Worth, TX (US); Daniel A. Gamache, Arlington, TX (US); Mark R. Hellberg, Arlington, TX (US); Stewart A. Noble, San Diego, CA (US); Andrew K. Shiau, San Diego, CA (US); David J. Thomas, San Diego, CA (US); John M. Yanni, Burleson, TX (US)

(73) Assignee: KALYPSYS, INC., Solana Beach, CA (US)

(21) Appl. No.: 13/370,756

(22) Filed: Feb. 10, 2012

Related U.S. Application Data
(62) Division of application No. 12/544,185, filed on Aug. 19, 2009, now Pat. No. 8,138,205.
Application No. 12/544,185 is a continuation-in-part of application No. 11/774,053, filed on Jul. 6, 2007, now Pat. No. 8,258,154.
(60) Provisional application No. 60/819,509, filed on Jul. 7, 2006.
(60) Provisional application No. 60/886,825, filed on Jan. 26, 2007.

(51) Int. Cl. A61K 31/4709; C07D 401/14; A61K 31/496; C07D 413/14; A61K 31/5377; A61K 31/551; A61P 27/02; A61P 27/14; A61P 27/04; A61P 27/06; A61P 29/00; A61P 9/10; A61P 25/00; A61P 37/08; C07D 401/12

(52) U.S. Cl. 514/235.2; 546/157; 514/312; 544/363; 514/253.07; 544/128; 540/575; 514/218

(57) ABSTRACT

The present invention relates to compounds and methods which may be useful as inhibitors of phosphodiesterase 4 (PDE4) for the treatment or prevention of inflammatory diseases and other diseases involving elevated levels of cytokines and proinflammatory mediators.

This application is a divisional of application Ser. No. 12/544,185, filed Aug. 19, 2009, which is a continuation-in-part of U.S. application Ser. No. 11/774,053, filed Jul. 6, 2007, which claims priority to U.S. Provisional Applications No. 60/819,509, filed Jul. 7, 2006, and No. 60/886,825, filed Jan. 26, 2007, the disclosures of which are hereby incorporated by reference as if written herein in its entirety.

Disclosed herein are new bicyclic heteroaryl compounds and compositions and their application as pharmaceuticals for the treatment of disease. Methods of inhibition of phosphodiesterase 4 (PDE4) activity in a human or animal subject are also provided for the treatment diseases such as inflammatory diseases and other diseases involving elevated levels of cytokines and proinflammatory mediators.

Chronic inflammation is a multi-factorial disease complication characterized by activation of multiple types of inflammatory cells, for example cells of lymphoid lineage (including T lymphocytes) and myeloid lineage (including granulocytes, macrophages, and monocytes). Proinflammatory mediators, including cytokines, such as tumor necrosis factor (TNF) and interleukin-1 (IL-1), are produced by these activated cells. Accordingly, an agent that suppresses the activation of these cells, or their production of proinflammatory cytokines, would be useful in the therapeutic treatment of inflammatory diseases and other diseases involving elevated levels of cytokines.

Cyclic adenosine monophosphate (cAMP) is a second messenger that mediates the biologic responses of cells to a wide range of extracellular stimuli. When the appropriate agonist binds to specific cell surface receptors, adenylate cyclase is activated to convert adenosine triphosphate (ATP) to cAMP. It is theorized that the agonist induced actions of cAMP within the cell are mediated predominately by the action of cAMP-dependent protein kinases. The intracellular actions of cAMP are terminated by either a transport of the nucleotide to the outside of the cell, or by enzymatic cleavage by cyclic nucleotide phosphodiesterases (PDEs), which hydrolyze the 3′-phosphodiester bond to form 5′-adenosine monophosphate (5′-AMP). 5′-AMP is an inactive metabolite.

The superfamily of PDEs is subdivided into two major classes, class I and class II, which have no recognizable sequence similarity. Class I includes all known mammalian PDEs and is comprised of 11 identified families that are products of separate genes. Some PDEs are highly specific for hydrolysis of cAMP (PDE4, PDE7, PDE8), some are highly cGMP-specific (PDE5, PDE6, PDE9), and some have mixed specificity (PDE1, PDE2, PDE3, PDE10, PDE11). All of the characterized mammalian PDEs are dimeric, but the importance of the dimeric structure for function in each of the PDEs is unknown.

The PDE4 subfamily is comprised of 4 members: PDE4A, PDE4B, PDE4C, and PDE4D. These enzymes possess N-terminal regulatory domains that presumably mediate dimerization, which results in optimally regulated PDE activity. In addition, activity is regulated via cAMP-dependent protein kinase phosphorylation sites in this upstream regulatory domain. PDE4 enzymes are broadly expressed and distributed.

Elevated levels of cAMP in human myeloid and lymphoid lineage cells are associated with the suppression of cell activation. The intracellular enzyme family of PDEs, therefore, regulates the level of cAMP in cells. PDE4 is a predominant PDE isotype in these cells, and is a major contributor to cAMP degradation. Accordingly, the inhibition of PDE function would prevent the conversion of cAMP to the inactive metabolite 5′-AMP and, consequently, maintain higher cAMP levels, and, accordingly, suppress cell activation.

PDE4 inhibitors have been shown to inhibit production of TNFα and partially inhibit IL-10 release by monocytes (see Semmler et al., Int. J. Immunopharmacol., 15, pp. 409-413, (1993); Molnar-Kimber et al., Mediators of Inflammation, 1, pp. 411-417, (1992)). PDE4 inhibitors also have been shown to inhibit the production of superoxide radicals from human polymorphonuclear leukocytes (see Verghese et al., J. Mol. Cell. Cardiol., 21 (Suppl. 2), S61 (1989); Nielson et al., J. Allergy Immunol., 86, pp. 801-808, (1990)); to inhibit the release of vasoactive amines and prostanoids from human basophils (see Peachell et al., J. Immunol., 148, pp. 2503-2510, (1992)); to inhibit respiratory bursts in eosinophils (see Dent et al., J. Pharmacol., 103, pp. 1339-1346, (1991)); and to inhibit the activation of human T-lymphocytes (see Robicsek et al., Biochem. Pharmacol., 42, pp. 869-877, (1991)).

Inflammatory cell activation and excessive or unregulated cytokine (e.g., TNFα and IL-1β) production are implicated in allergic, autoimmune, and inflammatory diseases and disorders, such as rheumatoid arthritis, osteoarthritis, gouty arthritis, spondylitis, thyroid associated ophthalmopathy, Behcet's disease, sepsis, septic shock, endotoxic shock, gram negative sepsis, gram positive sepsis, toxic shock syndrome, asthma, chronic bronchitis, adult respiratory distress syndrome, chronic pulmonary inflammatory disease, such as chronic obstructive pulmonary disease, silicosis, pulmonary sarcoidosis, reperfusion injury of the myocardium, brain, and extremities, fibrosis, cystic fibrosis, keloid formation, scar formation, atherosclerosis, transplant rejection disorders, such as graft vs. host reaction and allograft rejection, chronic glomerulonephritis, lupus, inflammatory bowel disease, such as Crohn's disease and ulcerative colitis, proliferative lymphocyte diseases, such as leukemia, ophthalmologic diseases such as dry eye and ocular pain resulting from inflammation or surgery, and inflammatory dermatoses, such as atopic dermatitis, psoriasis, and urticaria.

Other conditions characterized by elevated cytokine levels include brain injury due to moderate trauma, cardiomyopathies, such as congestive heart failure, cachexia, cachexia secondary to infection or malignancy, cachexia secondary to acquired immune deficiency syndrome (AIDS), ARC (AIDS related complex), fever myalgias due to infection, cerebral malaria, osteoporosis and bone resorption diseases, keloid formation, scar tissue formation, and pyrexia.

Additionally, several properties of TNFα, such as stimulation of collagenases, stimulation of angiogenesis in vivo, stimulation of bone resorption, and an ability to increase the adherence of tumor cells to endothelium, are consistent with a role for TNF in the development and metastatic spread of cancer in the host. TNFα recently has been directly implicated in the promotion of growth and metastasis of tumor cells (see Orosz et al., J. Exp. Med., 177, pp. 1391-1398, (1993)).

Investigators have shown considerable interest in the use of PDE4 inhibitors as anti-inflammatory agents. Early evidence indicates that PDE4 inhibition has beneficial effects on a variety of inflammatory cells such as monocytes, macrophages, T-cells of the Th-1 lineage, and granulocytes. The synthesis and/or release of many proinflammatory mediators, such as cytokines, lipid mediators, superoxide, and biogenic amines, such as histamine, have been attenuated in these cells by the action of PDE4 inhibitors. The PDE4 inhibitors also affect other cellular functions including T-cell proliferation, granulocyte transmigration in response to chemotoxic substances, and integrity of endothelial cell junctions within the vasculature.

The design, synthesis, and screening of various PDE4 inhibitors have been reported. Methylxanthines, such as caffeine and theophylline, were the first PDE inhibitors discovered, but these compounds are nonselective with respect to which PDE is inhibited. The drug rolipram, an antidepressant agent, was one of the first reported specific PDE4 inhibitors, with a reported IC₅₀ of about 200 nM with respect to inhibiting recombinant human PDE4.

Investigators have continued to search for PDE4 inhibitors that are more selective with respect to inhibiting PDE4, that have a lower IC₅₀ than rolipram, and that avoid the undesirable central nervous system (CNS) side effects, such as retching, vomiting, and sedation, associated with the administration of rolipram. In addition, several companies are now undertaking clinical trials of other PDE4 inhibitors. However, problems relating to efficacy and adverse side effects, such as emesis and central nervous system disturbances, remain unsolved.

Accordingly, compounds that selectively inhibit PDE4, and that reduce or eliminate the adverse side effects associated with prior PDE4 inhibitors, would be useful in the treatment of allergic and inflammatory diseases, and other diseases associated with excessive or unregulated production of cytokines, such as TNF. In addition, selective PDE4 inhibitors would be useful in the treatment of diseases that would benefit from elevated cAMP levels or reduced PDE4 function in a particular target tissue.

Novel compounds and pharmaceutical compositions, certain of which have been found to inhibit PDE4 have been discovered, together with methods of synthesizing and using the compounds including methods for the treatment of PDE4-mediated diseases in a patient by administering the compounds.

In certain embodiments of the present invention, compounds have structural Formula I:

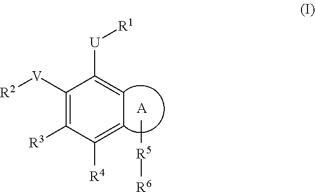

wherein:

A is an optionally substituted saturated or unsaturated hydrocarbon chain or a heteroatom-comprising hydrocarbon chain having from 3 to 5 atoms, forming a five- to seven-membered ring;

U, and V are independently selected from the group consisting of O, N(R⁷) and SO_{q};

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxy, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 0-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted;

R³ and R⁴ are each independently selected from the group consisting of hydrogen, halogen, alkoxy and lower alkyl;

R⁵ is selected from the group consisting of —(CR⁸R⁹)ₘW(CR¹⁰R¹¹)ₙ— and —(CR¹²R¹³)ₚ—;

m, n, and q are each independently 0, 1 or 2;

p is 1 or 2;

W is selected from the group consisting of O, N(R⁷), C(O)N(R⁷), and SO_{q};

R⁶ is selected from the group consisting of carboxy, alkylcarboxy, amido, aryl, heteroaryl, cycloalkyl, heterocycloalkyl, alkyl, heteroalkyl, acyl, and hydroxamic acid, any of which may be optionally substituted;

R⁷, R⁸, R⁹, R¹⁰, R¹¹, R¹², and R¹³ are each independently selected from the group consisting of hydrogen, halogen, hydroxyl, lower alkyl, hydroxyalkyl, haloalkyl, alkoxy, haloalkoxy, amino, aminoalkyl, and aminoalkoxy; and hydrogen and optionally substituted lower alkyl.

Certain compounds disclosed herein may possess useful PDE4 inhibiting activity, and may be used in the treatment or prophylaxis of a disease or condition in which PDE4 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting PDE4. Other embodiments provide methods for treating a PDE4-mediated disorder in a patient in need of such treatment, comprising administering to said patient a therapeutically effective amount of a compound or composition according to the present invention. Related embodiments disclose the use of certain compounds disclosed herein as therapeutic agents, for example, in treating inflammatory diseases and other diseases involving elevated levels of cytokines and proinflammatory mediators. Also provided is the use of certain compounds disclosed herein for use in the manufacture of a medicament for the treatment of a disease or condition ameliorated by the inhibition of PDE4.

In certain embodiments, the compounds have structural Formula II:

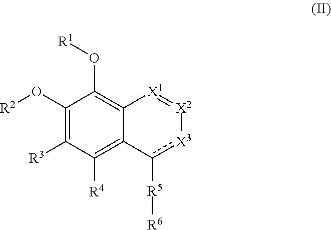

wherein:

X¹ is NR¹⁴

X² is selected from the group consisting of C(O), (CR¹⁵R¹⁶) and null;

X³ is selected from the group consisting of C(O), N(R¹⁷) and (CR¹⁸R¹⁹);

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxy, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 0-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted;

R³ and R⁴ are independently selected from the group consisting of hydrogen, halogen, alkoxy and lower alkyl;

R⁵ is selected from the group consisting of —(CR⁸R⁹)ₘW(CR¹⁰R¹¹)ₙ— and —(CR¹²R¹³)ₚ—;

m, n, and q are each independently 0, 1 or 2;

p is 1 or 2;

W is selected from the group consisting of O, N(R⁷), C(O)N(R⁷), and SO_{q};

R⁶ is selected from the group consisting of carboxy, alkylcarboxy, amido, aryl, heteroaryl, cycloalkyl, heterocycloalkyl, alkyl, heteroalkyl, acyl, and hydroxamic acid, any of which may be optionally substituted;

R⁸, R⁹, R¹⁰, R¹¹, R¹² and R¹³ are each independently selected from the group consisting of hydrogen and optionally substituted lower alkyl;

R¹⁴ and R¹⁷ are each independently selected from the group consisting of hydrogen, halogen, hydroxyl, lower alkyl, hydroxyalkyl, haloalkyl, aminoalkyl, and a bond; and

R¹⁵, R⁶, R¹⁸, and R¹⁹ are each independently selected from the group consisting of hydrogen, halogen, lower alkyl, haloalkyl, alkoxy, haloalkoxy, amino, aminoalkyl, aminoalkoxy, and null.

In certain further embodiments, compounds of the present invention have structural formula III

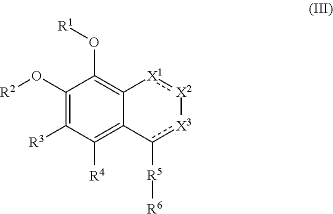

wherein:

X¹ is NR¹⁴

X² is selected from the group consisting of C(O) and null;

X³ is selected from the group consisting of C(O) and (CR¹⁸R¹⁹);

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 1-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted;

R³ and R⁴ are independently selected from the group consisting of hydrogen, halogen, alkoxy and lower alkyl;

R⁵ is selected from the group consisting of —(CR⁸R⁹)ₘW(CR¹⁰R¹¹)ₙ— and —(CR¹²R¹³)ₚ—;

m, n, and q are each independently 0, 1 or 2;

p is 1 or 2;

W is selected from the group consisting of O, N(R⁷), C(O)N(R⁷), and SO_{q};

R⁶ is selected from the group consisting of carboxyl, alkylcarboxy, amido, aryl, heteroaryl, cycloalkyl, heterocycloalkyl, alkyl, heteroalkyl, acyl, and hydroxamic acid, any of which may be optionally substituted;

R⁸, R⁹, R¹⁰, R¹¹, R¹² and R¹³ are each independently selected from the group consisting of hydrogen and optionally substituted lower alkyl;

R⁷ and R¹⁴ are independently selected from the group consisting of hydrogen, halogen, hydroxyl, lower alkyl, hydroxyalkyl, haloalkyl, and aminoalkyl; and

R⁵ and R¹⁶ are each independently selected from the group consisting of hydrogen, halogen, lower alkyl, haloalkyl, alkoxy, haloalkoxy, amino, aminoalkyl, aminoalkoxy, and null.

In certain embodiments, compounds of the present invention have structural Formula IV

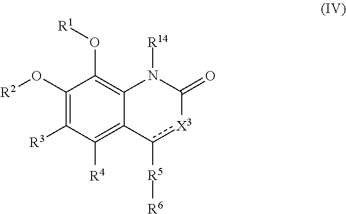

wherein:

X³ is (CR¹⁸R¹⁹);

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 1-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted;

R³ and R⁴ are each independently selected from the group consisting of hydrogen, halogen, alkoxy, and lower alkyl;

R⁵ is selected from the group consisting of —(CR⁸R⁹)ₘW(CR¹⁰R¹¹)ₙ— and —(CR¹²R¹³)ₚ—;

m, n, and q are each independently 0, 1 or 2;

p is 1 or 2;

W is selected from the group consisting of O, N(R⁷), C(O)N(R⁷), and SO_{q};

R⁶ is selected from the group consisting of carboxyl, alkylcarboxy, amido, aryl, heteroaryl, cycloalkyl, heterocycloalkyl, alkyl, heteroalkyl, acyl, and hydroxamic acid, any of which may be optionally substituted;

R⁸, R⁹, R¹⁰, R¹¹, R¹², and R¹³ are each independently selected from the group consisting of hydrogen and optionally substituted lower alkyl;

R⁷ and R¹⁴ are independently selected from the group consisting of hydrogen, halogen, hydroxyl, lower alkyl, hydroxyalkyl, haloalkyl, and aminoalkyl; and

R¹⁸ and R¹⁹ are each independently selected from the group consisting of hydrogen, halogen, lower alkyl, haloalkyl, alkoxy, haloalkoxy, amino, aminoalkyl, aminoalkoxy, and a null.

In further embodiments, compounds of the present invention have structural Formula V

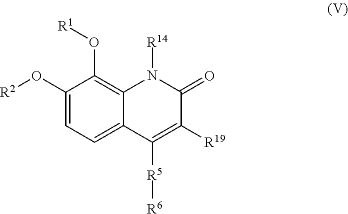

wherein:

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 1-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted;

R⁵ is selected from the group consisting of —(CR⁸R⁹)ₘW(CR¹⁰R¹¹)ₙ— and —(CR¹²R¹³)ₚ—;

m, n, and q are each independently 0, 1 or 2;

p is 1 or 2;

W is selected from the group consisting of O, N(R⁷), C(O)N(R⁷), and SO_{q};

R⁶ is selected from the group consisting of carboxyl, alkylcarboxy, amido, aryl, heteroaryl, cycloalkyl, heterocycloalkyl, alkyl, heteroalkyl, acyl, and hydroxamic acid, any of which may be optionally substituted;

R⁷ and R¹⁴ are independently selected from the group consisting of hydrogen, halogen, hydroxyl, lower alkyl, hydroxyalkyl, haloalkyl, and aminoalkyl; and

R¹⁹ is selected from the group consisting of hydrogen, halogen, lower alkyl and haloalkyl.

In yet further embodiments are provided compounds wherein R⁷ and R¹⁴ are independently selected from the group consisting of hydrogen, halogen and optionally substituted lower alkyl

In yet further embodiments are provided compounds wherein R⁶ is selected from the group consisting of aryl and heteroaryl, either of which may be optionally substituted;

In yet further embodiments are provided compounds wherein R¹⁹ is hydrogen.

In yet further embodiments are provided compounds wherein R⁶ is selected from the group consisting of phenyl, pyridine, pyrimidine, pyridazine, and pyrazine, any of which may be optionally substituted.

In yet further embodiments are provided compounds wherein R⁶ is selected from the group consisting of phenyl, pyridine, and pyrimidine, any of which may be optionally substituted.

In yet further embodiments are provided compounds wherein R¹⁴ is hydrogen.

In yet further embodiments are provided compounds wherein:

R⁵ is —(CR⁸R⁹)ₘW(CR¹⁰R¹¹)ₙ—;

m and n are both 0;

W is N(R⁷); and

R⁷ is hydrogen.

In yet further embodiments are provided compounds wherein R⁶ has the formula

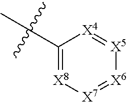

wherein:

X⁴ is CR²⁰ or N;

X⁵ is CR²¹ or N;

X⁶ is CR²² or N;

X⁷ is CR²³ or N;

X⁸ is CR²⁴ or N;

wherein no more than two of X⁴-X⁸ may be N; and

R²⁰, R²¹, R²², R²³ and R²⁴ are independently selected from the group consisting of hydrogen, halogen, hydroxyl, lower alkyl, lower alkoxy, lower haloalkyl, lower haloalkoxy, amino, and carboxyl.

In yet further embodiments are provided compounds wherein R⁶ has a formula selected from the group consisting of

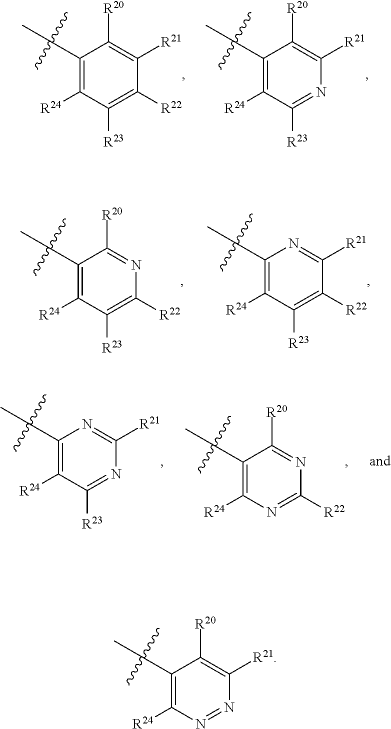

In certain embodiments are provided compounds wherein R² is lower alkyl.

In further embodiments, R² is methyl.

In yet further embodiments are provided compounds wherein:

R¹ is selected from the group consisting of —(CH₂)ₛG¹G²G³, lower alkyl, cycloalkyl, cycloalkylalkyl, and heterocycloalkyl, any of which may be optionally substituted;

s is 1-6;

G¹ is selected from the group consisting of amino, amido, and null;

G² is selected from the group consisting of alkoxy, aryl, halo, heterocycloalkyl, and null, any of which may be optionally substituted; and

G³ is selected from the group consisting of alkyl, carboxyl, and null, any of which may be optionally substituted.

In further embodiments, compounds of the present invention have structural Formula VI

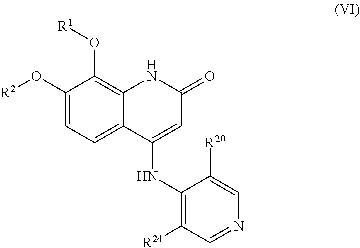

wherein:

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, heteroaryl, heteroaralkyl, hydrogen and hydroxyalkyl, any of which may be optionally substituted;

s is 1-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted; and

R²⁰ and R²⁴ are independently selected from the group consisting of hydrogen, halogen, hydroxyl, lower alkyl, lower alkoxy, lower haloalkyl, lower haloalkoxy, amino, and carboxyl.

In certain embodiments, R²⁰ and R²⁴ are independently selected from the group consisting of hydrogen, halogen, and lower alkyl.

In certain embodiments are provided compounds wherein:

R¹ and R² are independently selected from the group consisting of —(CH₂)ₛG¹G²G³, acyl, acylalkyl, C₁-C₁₀ alkyl, aminoalkyl, alkenyl, alkynyl, carboxyalkyl, heteroalkyl, cycloalkyl, cycloalkylalkyl, heterocycloalkyl, heterocycloalkylalkyl, aralkyl, aryl, heteroaryl, and heteroaralkyl, any of which maybe optionally substituted; and

G² is selected from the group consisting of alkyl, alkoxy, amino, aryl, halo, heterocycloalkyl, heteroaryl, carboxylalkylamino, an amino acid, and null, any of which may be optionally substituted with a substituent selected from the group consisting of lower alkyl, amino, alkylamino, aminoalkylamino, hydroxyalkylamino, cycloalkyl, and heterocycloalkyl

In other embodiments, R²⁰ and R²⁴ are independently selected from the group consisting of halogen and lower alkyl.

In certain embodiments, R² is lower alkyl.

In further embodiments are provided compounds wherein:

R¹ is selected from the group consisting of —(CH₂)ₛG¹G²G³, lower alkyl, cycloalkyl, cycloalkylalkyl, and heterocycloalkyl, any of which may be optionally substituted;

s is 1-6;

G¹ is selected from the group consisting of amino, amido, and null;

G² is selected from the group consisting of alkoxy, amino, aryl, halo, heterocycloalkyl, and null, any of which may be optionally substituted; and

G³ is selected from the group consisting of alkyl, carboxyl, and null, any of which may be optionally substituted.

In further embodiments, R² is methyl.

In yet further embodiments, R¹ is —(CH₂)ₛG².

In certain embodiments, s is 6.

In other embodiments, R²⁰ and R²⁴ are independently selected from the group consisting of halogen and methyl.

In yet other embodiments, R²⁰ and R²⁴ are independently selected from the group consisting of chlorine and methyl.

In further embodiments, G² is selected from the group consisting of amino and heterocycloalkyl.

In further embodiments, G² is selected from the group consisting of a five-, six, and seven-membered heterocycloalkyl containing one or two nitrogens.

In yet further embodiments, G² has a formula selected from the group consisting of

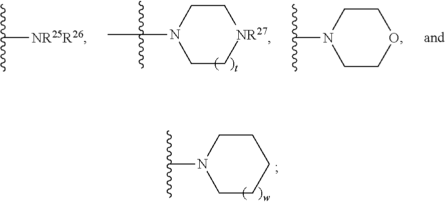

t is 1 or 2;

w is 0 or 1;

R²⁵ and R²⁶ are independently selected from the group consisting of hydrogen, methyl, —(CH₂)ₓOH, and —(CH₂)₃N(CH₃)₂;

x is 2 or 3; and

R²⁷ is selected from the group consisting of hydrogen and methyl.

In yet further embodiments, G² has a formula selected from the group consisting of

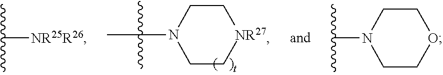

and

R²⁵ and R²⁶ are independently selected from the group consisting of methyl and —(CH₂)ₓOH.

In certain embodiments, R²⁰ and R²⁴ are each chlorine.

In other embodiments, G² has the formula

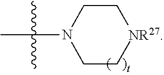

In certain embodiments are provided compounds wherein R²⁷ is methyl.

In other embodiments, t is 1.

In yet other embodiments, t is 2.

In further embodiments, G² has the formula selected from the group consisting of

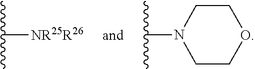

In certain embodiments, G² has the formula

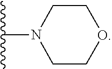 [SMILES: *N1CCOCC1]

In certain embodiments, G² is —NR²⁵R²⁶.

In other embodiments

R²⁵ is methyl; and

R²⁶ is and —(CH₂)ₓOH.

In yet other embodiments, x is 2.

In further embodiments, R²⁵ and R²⁶ are each methyl.

In further embodiments, R²⁰ and R²⁴ are each methyl.

In certain embodiments, compounds of the present invention have structural Formula VII

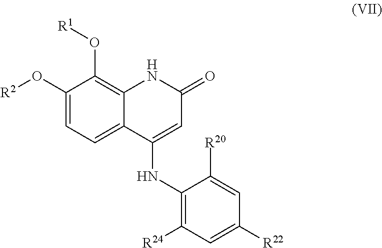

wherein:

R¹ and R² are independently selected from the group consisting of hydrogen, —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, hydrogen and hydroxyalkyl, any of which may be optionally substituted;

s is 1-8;

G¹ is selected from the group consisting of alkoxy, amino, amido, carbonyl, hydroxy, ether, an amino acid, and null;

G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;

G³ is selected from the group consisting of alkyl, alkoxy, amino, hydroxy, ether, carboxyl, hydroxamic acid, an amino acid, phosphonate, phosphoamide, and null, any of which may be optionally substituted; and

R²⁰, R²², and R²⁴ are independently selected from the group consisting of hydrogen, halogen, hydroxyl, lower alkyl, lower alkoxy, lower haloalkyl, lower haloalkoxy, amino, and carboxyl.

Compounds of the present invention may be selective amongst the PDE4 isoforms PDE4A, PDE4B, PDE4C, and PDE4D in various ways. For example, compounds described herein may be selective for PDE4B and PDE4D over the other two isoforms, be a pan-inhibitor of all the isoforms, or be selective for only one isoform. In certain embodiments, compounds of the present invention may be selective for PDE4B over other isoforms.

The present invention also relates to a method of inhibiting at least one PDE4 function comprising the step of contacting the PDE4 with a compound of Formula I, as described herein. The cell phenotype, cell proliferation, activity of PDE4, change in biochemical output produced by active PDE4, expression of PDE4, or binding of PDE4 with a natural binding partner may be monitored. Such methods may be modes of treatment of disease, biological assays, cellular assays, biochemical assays, or the like.

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched-chain hydrocarbon group having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, hydroxyalkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “amino acid”, as used herein, alone or in combination, refers to a —NHCHRC(O)O— group, which may be attached to the parent molecular moiety to give either an N-terminus or C-terminus amino acid, wherein R is independently selected from the group consisting of hydrogen, alkyl, aryl, heteroaryl, heterocycloalkyl, aminoalkyl, amido, amidoalkyl, carboxyl, carboxylalkyl, guanidinealkyl, hydroxyl, thiol, and thioalkyl, any of which themselves may be optionally substituted. The term C-terminus, as used herein, alone or in combination, refers to the parent molecular moiety being bound to the amino acid at the amino group, to give an amide as described herein, with the carboxyl group unbound, resulting in a terminal carboxyl group, or the corresponding carboxylate anion. The term N-terminus, as used herein, alone or in combination, refers to the parent molecular moiety being bound to the amino acid at the carboxyl group, to give an ester as described herein, with the amino group unbound resulting in a terminal secondary amine, or the corresponding ammonium cation. In other words, C-terminus refers to —NHCHRC(O)OH or to —NHCHRC(O)O⁻ and N-terminus refers to H₂NCHRC(O)O— or to H₃N⁺CHRC(O)O—.

The term “aryl”, as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′ group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “guanidine”, as used herein, alone or in combination, refers to —NHC(═NH)NH₂, or the corresponding guanidinium cation.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogen atoms are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, methylpiperazinyl, N-methylpiperazinyl, pyrrolidinyl, tetrahydropyridinyl, piperidinyl, thiomorpholinyl, diazepanyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “hydroxamic acid”, as used herein, alone or in combination, refers to —C(═O)NHOH, wherein the parent molecular moiety is attached to the hydroxamic acid group by means of the carbon atom.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphonate,” as used herein, alone or in combination, refers to a —P(═O)(OR)₂ group, wherein R is selected from the group consisting of alkyl and aryl. The term “phosphonic acid”, as used herein, alone or in combination, refers to a —P(═O)(OH)₂ group.

The term “phosphoramide”, as used herein, alone or in combination, refers to a —P(═O)(NR)₃ group, with R as defined herein.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethoxy” refers to a X₃CO— group where X is a halogen.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. When any one or more of G¹, G², and G³ of —(CH₂)ₛG¹G²G³ is designated to be “null”, said group condenses to either a bond if it occupies an interior position (as with G¹ and G²), or is absent if it occupies a terminal position (as with G³). Thus, for example, if G¹ and G³ are both null, then —(CH₂)ₛG¹G²G³ condenses to —(CH₂)ₛG². If G² and G³ are both null, then —(CH₂)ₛG¹G²G³ condenses to —(CH₂)ₛG¹. Similarly, if G¹ and G² are both null, then —(CH₂)ₛG¹G²G³ condenses to —(CH₂)ₛG³. When s is designated to be 0, then the (CH₂)ₛ portion of —(CH₂)ₛG¹G²G³ collapses to a bond connecting O to G¹G²G³. Each of G¹, G², and G³ are not meant to be null simultaneously and only two of G¹, G², and G³ may be null at once.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PDE4 inhibitor” is used herein to refer to a compound that exhibits an IC₅₀ with respect to PDE4 activity of no more than about 100 μM and more typically not more than about 50 μM, as measured in the PDE4 assay described generally hereinbelow. “IC₅₀” is that concentration of inhibitor which reduces the activity of an enzyme (e.g., PDE4) to half-maximal level. Certain representative compounds of the present invention have been discovered to exhibit inhibition against PDE4. In certain embodiments, compounds will exhibit an IC₅₀ with respect to PDE4 of no more than about 10 μM; in further embodiments, compounds will exhibit an IC₅₀ with respect to PDE4 of no more than about 5 μM; in yet further embodiments, compounds will exhibit an IC₅₀ with respect to PDE4 of not more than about 1 μM, as measured in the PDE4 assay described herein. In yet further embodiments, compounds will exhibit an IC₅₀ with respect to PDE4 of not more than about 200 nM.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations of the present invention may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected on the basis of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol®CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

For ophthalmic, otic, or nasal administration, the formulation may be a solution, a suspension, or a gel. In preferred aspects, the formulations are for topical application to the eye, nose, or ear in aqueous solution in the form of drops. The term “aqueous” typically denotes an aqueous formulation wherein the formulation is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts any preservative from the formulation as it is delivered, such devices being known in the art.

For ophthalmic disorders, components of the invention may be delivered to the eye as a concentrated gel or a similar vehicle, or as dissolvable inserts that are placed beneath the eyelids.

The formulations of the present invention that are adapted for topical administration to the eye are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

In certain ophthalmic embodiments, the compositions of the present invention are formulated with one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain formulations of the present invention may be used with contact lenses or other ophthalmic products.

In certain embodiments, formulations are prepared using a buffering system that maintains the formulation at a pH of about 4.5 to a pH of about 8. A most preferred formulation pH is from 7 to 8.

In certain embodiments, a formulation of the present invention is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof), and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with: H₁ antihistaminics, such as olopatadine hydrochloride and anti-infectives, such as moxifloxacin, gatifloxacin, finafloxacin, ciprofloxacin, lomefloxacin, levofloxacin, and norfloxacin. In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PDE4-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PDE4-mediated disorders.

Compounds of the subject invention may be useful in treating PDE4-mediated disease, disorders and conditions. In certain embodiments, said compounds may find use in treating acute and chronic pain and inflammation. The compounds of the present invention may be useful to treat patients with neuropathy, neuropathic pain, or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds may also be useful in the treatment of pain associated with acute herpes zoster (shingles), postherpetic neuralgia (PHN), and associated pain syndromes such as ocular pain. The compounds may further be useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds may also be useful for the treatment of pain-related disorders such as tactile allodynia and hyperalgesia. The pain may be somatogenic (either nociceptive or neuropathic), acute and/or chronic. The PDE4 inhibitors of the subject invention may also be useful in conditions where NSAIDs, morphine or fentanyl opiates and/or other opioid analgesics would traditionally be administered.

Furthermore, compounds of the subject invention may be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods of the present invention may be useful in the treatment or prevention of drug withdrawal symptoms, for example treatment or prevention of symptoms of withdrawal from opiate, alcohol, or tobacco addiction.

In addition, compounds of the subject invention may be used to treat insulin resistance and other metabolic disorders such as atherosclerosis that are typically associated with an exaggerated inflammatory signaling.

The present invention encompasses therapeutic methods using novel selective PDE4 inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; asthma-related diseases such as airway hyperreactivity and small airway disease; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchiolitis, bronchioectasis, cystic fibrosis, pigeon fancier's disease, farmer's lung, acute respiratory distress syndrome, pneumonia, pneumonitis, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus, hypoxia, dyspnea, hypercapnea, hyperinflation, hypoxemia, and cough. Further, compounds disclosed herein would find use in the treatment of allergic disorders such as delayed type hypersensitivity reaction, allergic contact dermatitis, allergic rhinitis, and chronic sinusitis.

Other disorders or conditions which may be treated by the compounds of the present invention include inflammation and related disorders. The compounds of the present invention may be useful as anti-inflammatory agents with the additional benefit of having significantly less harmful side effects. The compounds may be useful to treat arthritis, including but not limited to rheumatoid arthritis, spondyloarthropathies, gouty arthritis, osteoarthritis, juvenile arthritis, acute rheumatic arthritis, enteropathic arthritis, neuropathic arthritis, psoriatic arthritis, reactive arthritis (Reiter's syndrome), and pyogenic arthritis, and autoimmune diseases, including systemic lupus erythematosus, hemolytic syndromes, autoimmune hepatitis, autoimmune neuropathy, vitiglio (autoimmune thyroiditis), Hashimoto's thyroiditis, anemias, myositis including polymyositis, alopecia greata, Goodpasture's syndrome, hypophytis, and pulmonary fibrosis.

The compounds may also be useful in treating osteoporosis and other related bone disorders.

These compounds may also be used to treat gastrointestinal conditions such as reflux esophagitis, diarrhea, inflammatory bowel disease, Crohn's disease, gastritis, irritable bowel syndrome, Graves' disease (hyperthyroidism), necrotizing enterocolitis, and ulcerative colitis. The compounds may also be used in the treatment of pulmonary inflammation, such as that associated with viral infections and cystic fibrosis.

In addition, compounds of invention may also be useful in organ transplant patients either alone or in combination with conventional immunomodulators. Examples of conditions to be treated in said patients include graft vs. host reaction (i.e., graft vs. host disease), allograft rejections (e.g., acute allograft rejection, and chronic allograft rejection), transplant reperfusion injury, and early transplantation rejection (e.g., acute allograft rejection).

Yet further, the compounds of the invention may be useful in the treatment of pruritis and vitaligo.

The compounds of the present invention may also be useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Langerhans' cell histiocytosis, glomerulonephritis, reperfusion injury, pancreatitis, interstitial cystitis, Behcet's syndrome, polymyositis, gingivitis, periodontis, hypersensitivity, swelling occurring after injury, ischemias including myocardial ischemia, cardiovascular ischemia, and ischemia secondary to cardiac arrest, cirrhosis, septic shock, endotoxic shock, gram negative sepsis, toxic shock syndrome, stroke, ischemia reperfusion injury, multi-organ dysfunction, restenosis including restenosis following coronary bypass surgery, and the like.

The compounds of the subject invention may also be useful for the treatment of certain diseases and disorders of the nervous system. Central nervous system disorders in which PDE4 inhibition may be useful include cortical dementias including Alzheimer's disease and mild cognitive impairment (MCI), central nervous system damage resulting from stroke, ischemias including cerebral ischemia (both focal ischemia, thrombotic stroke and global ischemia (for example, secondary to cardiac arrest), and trauma. Neurodegenerative disorders in which PDE4 inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Parkinson's Disease, amyotrophic lateral sclerosis (ALS), Korsakoff's syndrome, and imbecility relating to a cerebral vessel disorder. Further disorders in which PDE4 inhibition might prove useful include neuropathies of the central and peripheral nervous system (including, for example, IgA neuropathy, membranous neuropathy and idiopathic neuropathy), chronic inflammatory demyelinating polyneuropathy, transverse myelitis, Gullain-Barre disease, encephalitis, and cancers of the nervous system. Disorders of CNS function in which PDE4 inhibitors may find use include sleeping disorders, schizophrenia, depression, depression or other symptoms associated with Premenstrual Syndrome (PMS), and anxiety.

Furthermore, the compounds of the present invention may also be useful in inhibiting PDE4 activity for the amelioration of systemic disorders including systemic hypotension associated with septic and/or toxic hemorrhagic shock induced by a wide variety of agents; as a therapy with cytokines such as TNF, IL-1 and IL-2; and as an adjuvant to short term immunosuppression in transplant therapy.

Still other disorders or conditions which may be treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, leukemia, lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods may also be used to treat the fibrosis which occurs with radiation therapy. The present compounds and methods may be used to treat subjects having adenomatous polyps, including those with familial adenomatous polyposis (FAP). Additionally, the present compounds and methods may be used to prevent polyps from forming in patients at risk of FAP.

The compounds disclosed herein can be used in the treatment of otic diseases and otic allergic disorders, including eustachian tube itching.

The compounds of the subject invention may be used in the treatment of ophthalmic diseases, such as ophthalmic allergic disorders, including allergic conjunctivitis, vernal conjunctivitis, vernal keratoconjunctivitis, and giant papillary conjunctivitis, dry eye, glaucoma, corneal neovascularization, optic neuritis, Sjogren's syndrome, retinal ganglion degeneration, ocular ischemia, retinitis, retinopathies, uveitis, ocular photophobia, and of inflammation and pain associated with acute injury to the eye tissue. Specifically, the compounds may be used to treat glaucomatous retinopathy and/or diabetic retinopathy. The compounds may also be used to treat post-operative inflammation or pain as from ophthalmic surgery such as cataract surgery and refractive surgery. In certain embodiments, the compounds of the present invention are used to treat an allergic eye disease selected from the group consisting of allergic conjunctivitis; vernal conjunctivitis; vernal keratoconjunctivitis; and giant papillary conjunctivitis.

Moreover, compounds of the subject invention may be used in the treatment of menstrual cramps, dysmenorrhea, premature labor, endometriosis, tendonitis, bursitis, skin-related conditions such as psoriasis, eczema, burns, sunburn, dermatitis, pancreatitis, hepatitis, lichen planus, scleritis, scleroderma, dermatomyositis, and the like. Other conditions in which the compounds of the subject invention may be used include diabetes (type I or type II), atherosclerosis, congestive heart failure, myocarditis, atherosclerosis, cerebral ischemia, angiogenesis, pulmonary hypertension, and aortic aneurysm.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Synthetic Methods for Preparing Compounds

The following schemes can be used to practice the present invention.

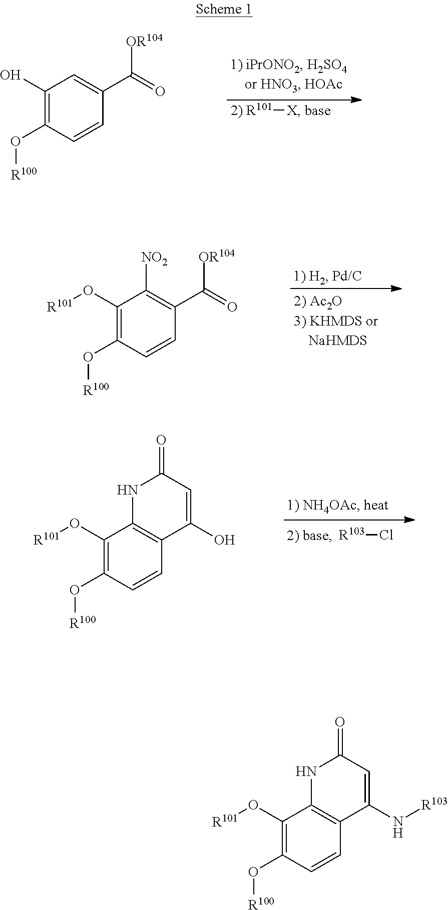

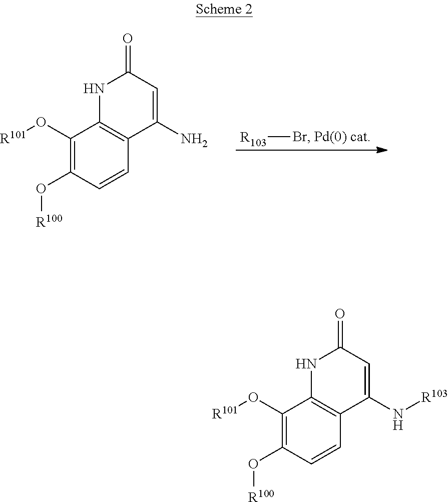

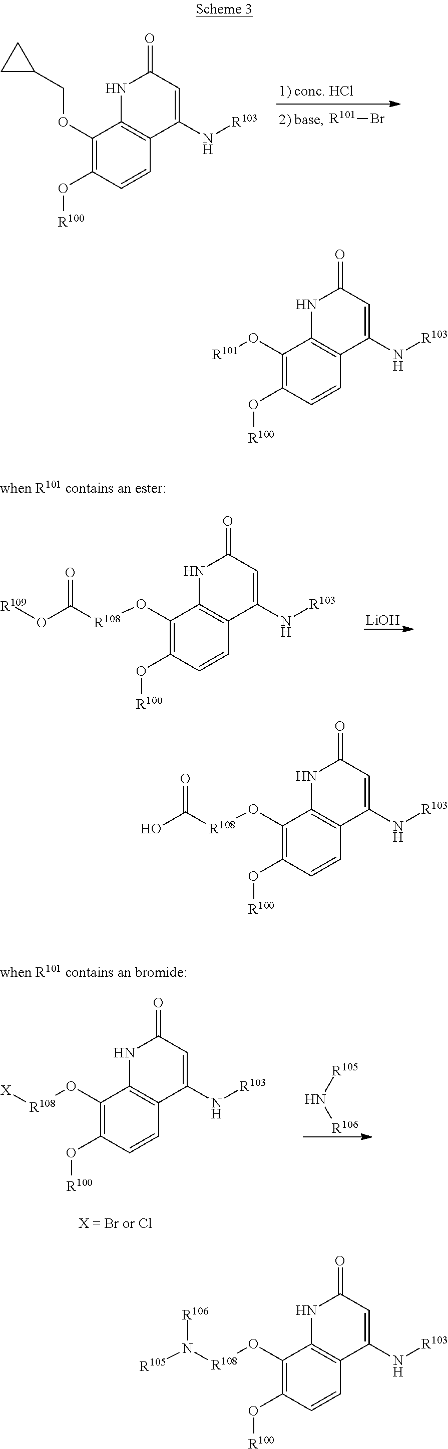

The invention is further illustrated by the following examples.

### EXAMPLE 1

### 8-(Cyclopentyloxy)-4-(3,5-dichloropyridin-4-ylamino)-7-methoxyquinolin-2(1H)-one

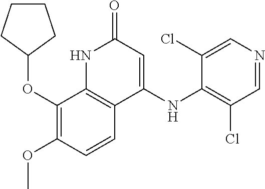 [SMILES: COc1ccc2c(Nc3c(Cl)cncc3Cl)cc(=O)[nH]c2c1OC1CCCC1]

### Step 1

Sulfuric acid (3.0 mL, 56 mmol) was added dropwise over 2 min to a solution of methyl 3-hydroxy-4-methoxybenzoate (3.64 g, 20 mmol), tetrabutylammonium hydrogensulfate (340 mg, 1.0 mmol), isopropyl nitrate (5.0 mL, 50 mmol), and dichloromethane (CH₂Cl₂) (40 mL) at 0° C. The reaction was allowed to warm to room temperature, maintained for 45 min, and then quenched with ice water (100 mL). This mixture was extracted with dichloromethane (250 mL×2). The combined extracts were dried, filtered, concentrated, and purified by silica gel chromatography (4:1→3:7; hexanes:ethyl acetate) to give methyl 3-hydroxy-4-methoxy-2-nitrobenzoate: MS (ESI): 228.4.

### Step 2

A mixture of methyl 3-hydroxy-4-methoxy-2-nitrobenzoate (1.14 g, 5.0 mmol), bromocyclopentane (0.7 mL, 6.5 mmol), potassium carbonate (2.0 g, 14 mmol), and anhydrous acetonitrile (15 mL) was refluxed under N₂. After 19 h, the reaction was allowed to cool to room temperature, concentrated, and purified by silica gel chromatography (4:1→3:2; hexanes:ethyl acetate) to give methyl 3-(cyclopentyloxy)-4-methoxy-2-nitrobenzoate: MS (ESI): 296.5.

### Step 3

A mixture of methyl 3-(cyclopentyloxy)-4-methoxy-2-nitrobenzoate (750 mg, 2.54 mmol), 10% Pd/C (50% H₂O, 500 mg, 0.23 mmol Pd), and methanol (10 mL) was stirred vigorously at 23° C. under an atmosphere of H₂. After 21 h, the mixture was filtered through Celite and concentrated to give methyl 2-amino-3-(cyclopentyloxy)-4-methoxybenzoate: MS (ESI): 266.5.

### Step 4

A solution of methyl 2-amino-3-(cyclopentyloxy)-4-methoxybenzoate (5.54 g, 20.9 mmol), acetic anhydride (21.0 mL, 222 mmol), and dioxane (35 mL) was heated at 40° C. under N₂ for 17 h and then allowed to cool to room temperature. Water (10 mL) was added, and after 15 min of stirring, the reaction was concentrated to give methyl 2-acetamido-3-(cyclopentyloxy)-4-methoxybenzoate: MS (ESI): 307.9.

### Step 5

A solution of methyl 2-acetamido-3-(cyclopentyloxy)-4-methoxybenzoate (6.42 g, 20.9 mmol) and anhydrous tetrahydrofuran (THF) (60 mL) was added via syringe pump (1 mL/min) to a solution of potassium bis(trimethylsilyl)amide (KHMDS) (150 mL, 0.5M PhMe, 75 mmol) at −78° C. under N₂. The reaction was allowed to slowly warm to room temperature as the bath expired overnight. After 17 h, the reaction was poured into ice/water (250 mL), let stir for 10 min, and the layers were separated. The aqueous was washed with ether (150 mL), acidified with conc. HCl (7 mL), and filtered to give 8-(cyclopentyloxy)-4-hydroxy-7-methoxyquinolin-2(1H)-one: MS (ESI): 276.0.

### Step 6

A mixture of 8-(cyclopentyloxy)-4-hydroxy-7-methoxyquinolin-2(1H)-one (350 mg, 1.3 mmol), ammonium acetate (4.0 g, 52 mmol), and anhydrous m-xylene (10 mL) was heated at 150° C. under N₂ for 48 h and then allowed to cool to room temperature. The mixture was concentrated to dryness, diluted with water (25 mL), stirred for 2 min, sonicated for 1 min, and then stirred for 5 min. The free-flowing solid was filtered, washed with water (25 mL), and pumped to dryness to give 4-amino-8-(cyclopentyloxy)-7-methoxyquinolin-2(1H)-one: MS (ESI): 274.9.

### Step 7

4-Amino-8-(cyclopentyloxy)-7-methoxyquinolin-2(1H)-one (340 mg, 1.24 mmol) was added in 3 portions over 5 minutes to a mixture of sodium hydride (NaH) (110 mg, 60%, 2.75 mmol) and DMSO (4 mL) at room temperature under N₂. After 5 min, 3,4,5-trichloropyridine (271 mg, 1.49 mmol) was added. After 19 h, more sodium hydride (100 mg, 60%, 2.5 mmol) was added followed by more 3,4,5-trichloropyridine (250 mg, 1.37 mmol). After an additional 4 h, the reaction was poured into 1M KH₂PO₄ (75 mL) and stirred for 5 min. The solids were filtered, washed with water (25 mL), and then purified by reverse-phase HPLC (3:7→1:0; acetonitrile:water). In some cases, additional purification by silica gel chromatography (1:0→9:1; dichlormethane:methanol) was needed to give 8-(cyclopentyloxy)-4-(3,5-dichloropyridin-4-ylamino)-7-methoxyquinolin-2(1H)-one: ¹H NMR (400 MHz, DMSO-d₆): δ 9.39 (s, 1H), 8.86 (s, 1H), 8.76 (s, 2H), 7.87 (d, 1H), 7.06 (d, 1H), 4.97 (m, 1H), 4.78 (s, 1H), 3.90 (s, 3H), 1.83-1.48 (m, 8H); MS (ESI): 419.7.

### EXAMPLE 2

### 8-(Cyclopentyloxy)-4-(3,5-dichloropyridin-4-yloxy)-7-methoxyquinolin-2(1H)-one

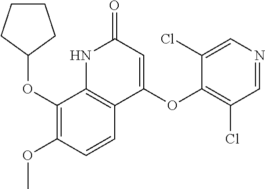 [SMILES: COc1ccc2c(Oc3c(Cl)cncc3Cl)cc(=O)[nH]c2c1OC1CCCC1]

The title compound was isolated from the purification of Example 1, Step 7. ¹H NMR (400 MHz, DMSO-d₆): δ 10.28 (s, 1H), 8.84 (s, 2H), 7.71 (d, 1H), 7.11 (d, 1H), 5.35 (s, 1H), 4.97 (m, 1H), 3.91 (s, 3H), 1.86-1.43 (m, 8H); MS (ESI): 420.7.

### EXAMPLE 3

### 8-(Cyclopropylmethoxy)-4-(3,5-dichloropyridin-4-ylamino)-7-methoxyquinolin-2(1H)-one

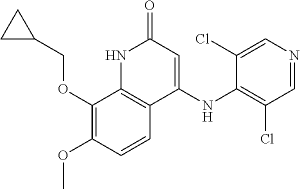 [SMILES: COc1ccc2c(Nc3c(Cl)cncc3Cl)cc(=O)[nH]c2c1OCC1CC1]

The title compound was prepared from methyl 3-hydroxy-4-methoxy-2-nitrobenzoate (Example 1, Step 1) and (bromomethyl)cyclopropane following the procedures outlined in Example 1. ¹H NMR (400 MHz, DMSO-d₆): δ 9.91 (s, 1H), 8.83 (s, 1H), 8.76 (s, 2H), 7.87 (d, 1H), 7.03 (d, 1H), 4.78 (s, 1H), 3.89 (s, 3H), 3.84 (d, 2H), 1.27 (m, 1H), 0.45 (m, 2H), 0.25 (m, 2H); MS (ESI): 405.8.

### EXAMPLE 4

### 8-(Cyclopentyloxy)-4-(3,5-dichloropyridin-4-ylamino)-7-methoxy-1-methylquinolin-2(1H)-one

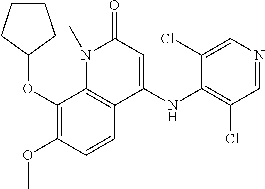 [SMILES: COc1ccc2c(Nc3c(Cl)cncc3Cl)cc(=O)n(C)c2c1OC1CCCC1]

### Step 1

A solution of methyl 2-acetamido-3-(cyclopentyloxy)-4-methoxybenzoate (1.6 g, 5.2 mmol, Example 1, Step 4) and anhydrous tetrahydrofuran (30 mL) was added over 5 min to a mixture of sodium hydride (230 mg, 5.75 mmol) and anhydrous tetrahydrofuran (10 mL) at 0° C. under N₂. The reaction was allowed to warm to room temperature and after 20 min, cooled back to 0° C. A solution of iodomethane (1.06 g, 0.0075 mol) and anhydrous tetrahydrofuran (10 mL) was added over 3 min. The reaction was allowed to warm to room temperature and after 30 min, concentrated. The residue was diluted with ethyl acetate (40 mL) and washed with brine (10 mL×2). The organic layer was dried, filtered, and concentrated to give methyl 3-(cyclopentyloxy)-4-methoxy-2-(N-methylacetamido)benzoate: MS (ESI): 322.1.

### Step 2

The title compound was prepared from methyl 3-(cyclopentyloxy)-4-methoxy-2-(N-methylacetamido)benzoate following the procedures outlined in Example 1. ¹H NMR (400 MHz, DMSO-d₆): δ 8.75 (s, 1H), 8.74 (s, 2H), 7.92 (d, 1H), 7.13 (d, 1H), 4.92 (s, 1H), 4.53 (m, 1H), 3.90 (s, 3H), 3.59 (s, 3H), 1.80-1.48 (m, 8H); MS (ESI): 433.9.

### EXAMPLE 5

### 8-(Cyclopentyloxy)-4-(3,5-dimethylpyridin-4-ylamino)-7-methoxyquinolin-2(1H)-one

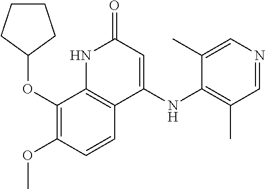 [SMILES: COc1ccc2c(Nc3c(C)cncc3C)cc(=O)[nH]c2c1OC1CCCC1]

The title compound was prepared from 4-bromo-3,5-dimethylpyridine and 4-amino-8-(cyclopentyloxy)-7-methoxyquinolin-2(1H)-one (Example 1, Step 6) following the procedure outlined in Example 8. ¹H NMR (400 MHz, DMSO-d₆): δ 9.08 (s, 1H), 8.44 (s, 1H), 8.40 (s, 2H), 7.88 (d, 1H), 7.03 (d, 1H), 4.96 (m, 1H), 4.51 (s, 1H), 3.90 (s, 3H), 2.13 (s, 6H), 1.80-1.54 (m, 8H); MS (ESI): 380.2.

### EXAMPLE 6

### 1,3-Dibromo-8-(cyclopentyloxy)-4-(3,5-dichloropyridin-4-ylamino)-7-methoxyquinolin-2(1H)-one

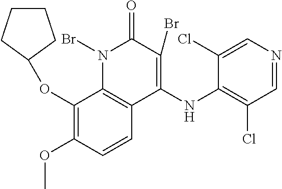 [SMILES: COc1ccc2c(Nc3c(Cl)cncc3Cl)c(Br)c(=O)n(Br)c2c1OC1CCCC1]

N-Bromosuccinimide (0.118 g, 0.61 mmol) was added to a solution of 4-(3,5-dichloropyridin-4-ylamino)-8-(cyclopentyloxy)-7-methoxyquinolin-2(1H)-one (0.100 g, 0.240 mmol, Example 1) and anhydrous dimethylformamide (2 mL) at 0° C. After 2 h at 0° C., the mixture was poured into water, and the precipitate was filtered to give 1,3-dibromo-8-(cyclopentyloxy)-4-(3,5-dichloropyridin-4-ylamino)-7-methoxyquinolin-2(1H)-one: ¹H NMR (400 MHz, DMSO-d₆): δ 10.5 (s, 1H), 8.59 (s, 2H), 6.76 (d, 1H), 6.61 (d, 1H), 4.87 (m, 1H), 3.80 (s, 3H), 1.82-1.51 (m, 8H); MS (ESI): 575.8.

### EXAMPLE 7

### 3-Bromo-8-(cyclopentyloxy)-4-(3,5-dichloropyridin-4-ylamino)-7-methoxyquinolin-2(1H)-one

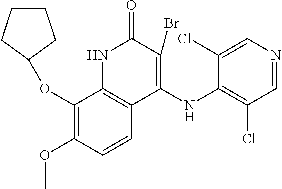 [SMILES: COc1ccc2c(Nc3c(Cl)cncc3Cl)c(Br)c(=O)[nH]c2c1OC1CCCC1]

N-Bromosuccinimide (0.104 g, 0.585 mmol) was added to a solution of 4-(3,5-dichloropyridin-4-ylamino)-8-(cyclopentyloxy)-7-methoxyquinolin-2(1H)-one (0.246 g, 0.585 mmol, Example 1) and anhydrous dimethylformamide (3 mL) at 0° C. After 4 h at 0° C., the mixture was poured into water, and the precipitate was filtered. Saturated sodium thiosulfate solution (1 mL) was added to a solution of the filter cake and methanol (5 mL). After the dibromo intermediate was consumed, as monitored by LCMS, the mixture was diluted with water and extracted with ethyl acetate. The organic extract was dried, filtered, and concentrated to give 3-bromo-8-(cyclopentyloxy)-4-(3,5-dichloropyridin-4-ylamino)-7-methoxyquinolin-2(1H)-one: ¹H NMR (400 MHz, DMSO-d₆): δ 10.56 (s, 1H), 8.88 (br s, 1H), 8.42 (s, 2H), 7.62 (d, 1H), 7.04 (d, 1H), 4.98 (m, 1H), 3.89 (s, 3H), 1.84-1.51 (m, 8H); MS (ESI): 497.8.

### EXAMPLE 8

### 8-(Cyclopropylmethoxy)-4-(3,5-dimethylpyridin-4-ylamino)-7-methoxyquinolin-2(1H)-one

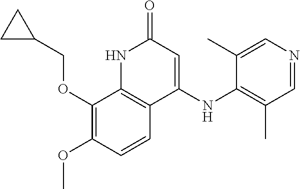 [SMILES: COc1ccc2c(Nc3c(C)cncc3C)cc(=O)[nH]c2c1OCC1CC1]

A solution of 4-bromo-3,5-dimethylpyridine (1.16 g, 6.21 mmol) and degassed toluene (5 mL) was added to a mixture of 4-amino-8-(cyclopropylmethoxy)-7-methoxyquinolin-2(1H)-one (1.94 g, 7.45 mmol, intermediate for Example 3), tris(dibenzylideneacetone)dipalladium(0) (Pd₂ (dba)₃), (0.28 g, 0.31 mmol), 2,2-dicyclohexylphosphorane triisopropylbiphenyl (0.59 g, 1.24 mmol), sodium tert-butoxide (1.20 g, 12.4 mmol), and degassed toluene (20 mL) under N₂. The mixture was heated at 110° C. for 2 h, allowed to cool to room temperature, sonicated until the solids broke up, and then filtered through Celite with ethyl acetate (500 mL). The filtrate was concentrated, purified by silica gel chromatography (0-8% methanol/dichloromethane), and then re-purified by reverse-phase HPLC (25→100% acetonitrile/H₂O) to give 8-(cyclopropylmethoxy)-4-(3,5-dimethylpyridin-4-ylamino)-7-methoxyquinolin-2(1H)-one: ¹H NMR (400 MHz, DMSO-d₆): δ 9.64 (s, 1H), 8.43 (s, 1H), 8.42 (s, 2H), 7.89 (d, 1H), 7.03 (d, 1H), 4.54 (s, 1H), 3.91 (s, 3H), 3.85 (d, 2H), 2.15 (s, 6H), 1.28 (m, 1H), 0.47 (m, 2H), 0.28 (m, 2H); MS (ESI): 365.8.

### EXAMPLE 9

### 8-(Cyclopentyloxy)-4-(3,5-dichloropyridin-4-ylamino)-1-(2-hydroxyethyl)-7-methoxyquinolin-2(1H)-one

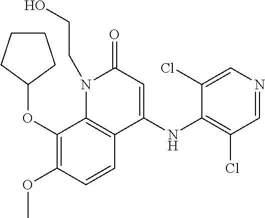 [SMILES: COc1ccc2c(Nc3c(Cl)cncc3Cl)cc(=O)n(CCO)c2c1OC1CCCC1]

### Step 1

Methyl 3-(cyclopentyloxy)-4-methoxy-2-(N-(2-(tetrahydro-2H-pyran-2-yloxy)ethyl)acetamido)benzoate was prepared from methyl 2-acetamido-3-(cyclopentyloxy)-4-methoxybenzoate (Example 1, Step 4) and 2-(2-bromoethoxy)-tetrahydro-2H-pyran following the procedure outlined in Example 4 (with the following exceptions: the solvent used was dimethylformamide, the reaction temperature was 33° C., and the reaction was conducted overnight). MS (ESI): 458.2 (M+Na).

### Step 2

8-(Cyclopentyloxy)-4-(3,5-dichloropyridin-4-ylamino)-7-methoxy-1-(2-(tetrahydro-2H-pyran-2-yloxy)ethyl)quinolin-2(1H)-one was prepared from methyl 3-(cyclopentyloxy)-4-methoxy-2-(N-(2-(tetrahydro-2H-pyran-2-yloxy)ethyl)acetamido)benzoate following the procedures outlined in Example 1. MS (ESI): 570 (M+Na).

### Step 3

para-Toluenesulfonic acid (172 mg, 1.0 mmol) was added to a solution of 8-(cyclopentyloxy)-4-(3,5-dichloropyridin-4-ylamino)-7-methoxy-1-(2-(tetrahydro-2H-pyran-2-yloxy)ethyl)quinolin-2(1H)-one (2.4 g, 4.4 mmol) and methanol (100 mL). After stirring overnight, the reaction was concentrated, diluted with dichloromethane (100 mL), and then washed with saturated NaHCO₃ (20 mL) followed by water (20 mL). The organic layer was dried, filtered, concentrated, and recrystallized from dichloromethane/petroleum ether (1:2) to give 8-(cyclopentyloxy)-4-(3,5-dichloropyridin-4-ylamino)-1-(2-hydroxyethyl)-7-methoxyquinolin-2(1H)-one: ¹H NMR (400 MHz, DMSO-d₆): δ 8.74 (s, 2H), 8.73 (s, 1H), 7.91 (d, 1H), 7.11 (d, 1H), 4.92 (s, 1H), 4.65 (t, 2H), 4.59 (m, 1H), 4.50 (t, 1H), 3.90 (s, 3H), 3.32 (m 2H), 1.80-1.48 (m, 8H); MS (ESI): 463.9.

### EXAMPLE 10

### 8-(Cyclopentyloxy)-4-(3,5-dichloropyridin-4-ylamino)-1-(2-(dimethylamino)ethyl)-7-methoxyquinolin-2(1H)-one

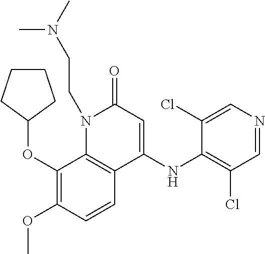 [SMILES: COc1ccc2c(Nc3c(Cl)cncc3Cl)cc(=O)n(CCN(C)C)c2c1OC1CCCC1]

### Step 1

Dess-Martin periodinane (933 mg, 2.2 mmol) was added to a solution of 8-(cyclopentyloxy)-4-(3,5-dichloropyridin-4-ylamino)-1-(2-hydroxyethyl)-7-methoxyquinolin-2(1H)-one (500 mg, 1.08 mmol, Example 9) and CH₂Cl₂ (30 mL) at 0° C. After 30 min, the mixture was allowed to warm to room temperature. When the starting material was consumed, the reaction was diluted with dichloromethane (25 mL), washed with aq. NaOH (20 mL×2), and then washed with water (20 mL). The organic layer was dried, filtered, concentrated, and purified by silica gel chromatography (1:2→1:1; ethyl acetate:petroleum ether) to give 2-(8-(cyclopentyloxy)-4-(3,5-dichloropyridin-4-ylamino)-7-methoxy-2-oxoquinolin-1(2H)-yl)acetaldehyde: MS (ESI): 462.2.

### Step 2

Titanium (IV) isopropoxide (496 mg, 1.74 mmol) and 2-(8-(cyclopentyloxy)-4-(3,5-dichloropyridin-4-ylamino)-7-methoxy-2-oxoquinolin-1(2H)-yl)acetaldehyde (400 mg, 0.87 mmol) were added sequentially to a solution of dimethylamine hydrochloride (144 mg, 1.74 mmol), triethylamine (176 mg, 1.74 mmol), and absolute ethanol (20 mL). After stirring overnight, sodium borohydride (50 mg, 1.3 mmol) was added. After an additional 3 h, the reaction was poured into aq. ammonia (30 mL) and then filtered—washing with dichloromethane. The filtrate was extracted with dichloromethane (50 mL×3). The combined extracts were dried, filtered, concentrated, and purified by reverse-phase HPLC to give 8-(cyclopentyloxy)-4-(3,5-dichloropyridin-4-ylamino)-1-(2-(dimethylamino)ethyl)-7-methoxyquinolin-2(1H)-one: ¹H NMR (400 MHz, DMSO-d₆): δ 8.75 (s, 1H), 8.73 (s, 2H), 7.91 (d, 1H), 7.12 (d, 1H), 4.94 (s, 1H), 4.71 (t, 2H), 4.58 (m, 1H), 3.90 (s, 3H), 2.22 (m, 2H), 2.04 (s, 6H), 1.80-1.50 (m, 8H); MS (ESI): 491.0.

### EXAMPLE 11

### 6-(4-(3,5-Dichloropyridin-4-ylamino)-7-methoxy-2-oxo-1,2-dihydroquinolin-8-yloxy)hexanoic acid

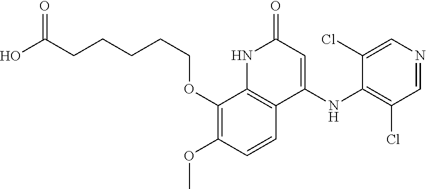 [SMILES: COc1ccc2c(Nc3c(Cl)cncc3Cl)cc(=O)[nH]c2c1OCCCCCC(=O)O]

### Step 1

Concentrated HCl (15 ml) was added to a solution of 8-(cyclopropylmethoxy)-4-(3,5-dichloropyridin-4-ylamino)-7-methoxyquinolin-2(1H)-one (1.2 g, 2.90 mmol, Example 3) and methanol (8 ml). The reaction mixture was heated at 50° C. for 4 h and then concentrated. The residue was neutralized with saturated NaHCO₃, filtered (washed with ethanol and ether), and dried to give 4-(3,5-dichloropyridin-4-ylamino)-8-hydroxy-7-methoxyquinolin-2(1H)-one: MS (ESI): 352.

### Step 2

Sodium hydride (54 mg, 1.35 mmol) was added to a solution of 4-(3,5-dichloropyridin-4-ylamino)-8-hydroxy-7-methoxyquinolin-2(1H)-one (380 mg, 1.08 mmol) and DMSO (20 ml) room temperature under N₂. After 1 h, ethyl 6-bromohexanoate (770 mg, 3.45 mmol) was added, and the mixture was heated at 30° C. for 5 h. The reaction was quenched with drops of water, poured into 0.5 M KH₂PO₄, and extracted with ethyl acetate (100 mL×3). The combined extracts were washed with water and then brine, dried, filtered, concentrated, and purified by silica gel chromatography (ethyl acetate:pet. ether) to give ethyl 6-(4-(3,5-dichloropyridin-4-ylamino)-7-methoxy-2-oxo-1,2-dihydroquinolin-8-yloxy)hexanoate: MS (ESI): 494.0.

### Step 3

Lithium hydroxide (2.0 mL, 1M, 2.0 mmol) was added to a solution of ethyl 6-(4-(3,5-dichloropyridin-4-ylamino)-7-methoxy-2-oxo-1,2-dihydroquinolin-8-yloxy)hexanoate (100 mg, 0.20 mmol), methanol (2 mL), and tetrahydrofuran (8 mL). The mixture was stirred for 24 h, quenched with 1N HCl (30 mL), and extracted with ethyl acetate (40 mL×2). The organic extracts were dried, filtered, and concentrated to give 6-(4-(3,5-dichloropyridin-4-ylamino)-7-methoxy-2-oxo-1,2-dihydroquinolin-8-yloxy)hexanoic acid: ¹H NMR (400 MHz, DMSO-d₆): δ 10.10 (s, 1H), 8.88 (s, 1H), 8.77 (s, 2H), 7.89 (d, 1H), 7.06 (d, 1H), 4.80 (s, 1H), 3.95 (t, 2H), 3.90 (s, 3H), 2.22 (t, 2H), 1.76 (m, 2H), 1.55 (m, 2H), 1.39 (m, 2H); MS (ESI): 465.8.

### EXAMPLE 12

### 4-(3,5-Dimethylpyridin-4-ylamino)-8-isobutoxy-7-methoxyquinolin-2(1H)-one

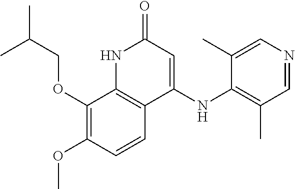 [SMILES: COc1ccc2c(Nc3c(C)cncc3C)cc(=O)[nH]c2c1OCC(C)C]

The title compound was prepared from methyl 3-hydroxy-4-methoxy-2-nitrobenzoate (Example 1, Step 1) and 1-bromo-2-methylpropane following the procedures outlined in Examples 1 & 8. ¹H NMR (400 MHz, DMSO-d₆): δ 9.39 (s, 1H), 8.42 (s, 1H), 8.39 (s, 2H), 7.89 (d, 1H), 7.03 (d, 1H), 4.51 (s, 1H), 3.89 (s, 3H), 3.74 (d, 2H), 2.13 (s, 6H), 2.13 (m, 1H), 0.97 (d, 6H); MS (ESI): 368.2.

### EXAMPLE 13

### 4-(3,5-Dichloropyridin-4-ylamino)-8-(4-(dimethylamino)butoxy)-7-methoxyquinolin-2(1H)-one

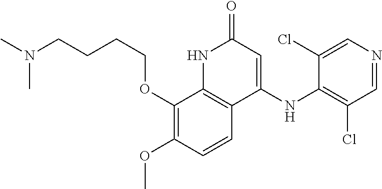 [SMILES: COc1ccc2c(Nc3c(Cl)cncc3Cl)cc(=O)[nH]c2c1OCCCCN(C)C]

### Step 1

8-(4-Bromobutoxy)-4-(3,5-dichloropyridin-4-ylamino)-7-methoxyquinolin-2(1H)-one was prepared from 4-(3,5-dichloropyridin-4-ylamino)-8-hydroxy-7-methoxyquinolin-2(1H)-one (Example 11, Step 1) and 1,4-dibromobutane following the procedure outlined in Example 11, Step 2. MS (ESI): 485.8.

### Step 2

A solution of dimethylamine (1.5 mL, 2M tetrahydrofuran, 3 mmol) was added to a solution of 8-(4-bromobutoxy)-4-(3,5-dichloropyridin-4-ylamino)-7-methoxyquinolin-2(1H)-one (146 mg, 0.3 mmol) and DMSO (3 mL). After 2.5 h, the reaction was poured into 10% K₂CO₃ (30 mL) and extracted with dichloromethane (40 mL×2). The combined extracts were dried, filtered, concentrated, and purified by silical gel chromatography (1:0→4:1; dichloromethane:methanol w/1% conc. NH₄OH solution) and then reverse-phase HPLC (1:9→1:1; acetonitrile:water) to give 4-(3,5-dichloropyridin-4-ylamino)-8-(4-(dimethylamino)butoxy)-7-methoxyquinolin-2(1H)-one: ¹H NMR (400 MHz, DMSO-d₆): δ 10.29 (s, 1H), 8.81 (s, 1H), 8.75 (s, 2H), 7.87 (d, 1H), 7.04 (d, 1H), 4.77 (s, 1H), 3.99 (t, 2H), 3.90 (s, 3H), 2.27 (t, 2H), 2.13 (s, 6H), 1.73 (m, 2H), 1.54 (m, 2H); MS (ESI): 451.0.

### EXAMPLE 14

### 4-(3,5-Dichloropyridin-4-ylamino)-8-(5-(dimethylamino)pentyloxy)-7-methoxyquinolin-2(1H)-one

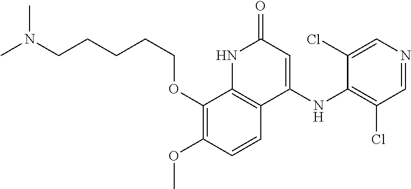 [SMILES: COc1ccc2c(Nc3c(Cl)cncc3Cl)cc(=O)[nH]c2c1OCCCCCN(C)C]

The title compound was prepared from 4-(3,5-dichloropyridin-4-ylamino)-8-hydroxy-7-methoxyquinolin-2(1H)-one (Example 11, Step 1), 1,5-dibromopentane, and dimethylamine following the procedures outlined in Example 13. ¹H NMR (400 MHz, DMSO-d₆): δ 9.91 (s, 1H), 8.83 (s, 1H), 8.75 (s, 2H), 7.87 (d, 1H), 7.04 (d, 1H), 4.77 (s, 1H), 3.96 (t, 2H), 3.90 (s, 3H), 2.19 (t, 2H), 2.10 (s, 6H), 1.76 (m, 2H), 1.48-1.32 (m, 4H) MS (ESI): 465.0.

### Intermediate 1

### Methyl 3-hydroxy-4-methoxy-2-nitrobenzoate

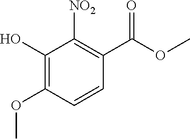 [SMILES: COC(=O)c1ccc(OC)c(O)c1[N+](=O)[O-]]

Nitric acid (95%, 42 g, 0.62 mol) was added dropwise (internal temperature kept below 5° C.) to a solution of methyl 3-hydroxy-4-methoxybenzoate (96.5%, 100 g, 0.53 mol) and acetic acid (250 mL) at 0° C. After 10 min at 0° C., a large amount of precipitate had formed and was collected by filtration. The filter cake was washed with water (100 mL×2), and then the crude product was recrystallized from methanol (40 mL) to give 33 g of methyl 3-hydroxy-4-methoxy-2-nitrobenzoate as a light yellow solid. ¹H NMR (400 MHz, DMSO-d₆): δ 10.64 (s, 1H), 7.49 (d, 1H), 7.22 (d, 1H), 3.93 (s, 3H), 3.76 (s, 3H); MS (ESI): 227.9.

### Intermediate 2

### 4-Amino-8-(cyclopropylmethoxy)-7-methoxyquinolin-2(1H)-one

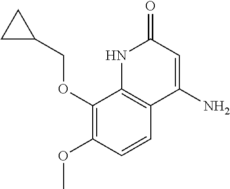 [SMILES: COc1ccc2c(N)cc(=O)[nH]c2c1OCC1CC1]

### Step 1

(Bromomethyl)cyclopropane (136 g, 1.01 mol) and potassium carbonate (173 g, 1.25 mol) were added to a solution of methyl 3-hydroxy-4-methoxy-2-nitrobenzoate (190 g, 837 mmol) and dimethylformamide (1 L). The mixture was stirred at room temperature overnight and then quenched with water/ice (3 L). The resulting solids were collected by filtration, washed with water (500 mL×3), and then dried in an oven to give 220 g of methyl 3-(cyclopropylmethoxy)-4-methoxy-2-nitrobenzoate as a yellow solid. MS (ESI): 281.9.

### Step 2

A mixture of methyl 3-(cyclopropylmethoxy)-4-methoxy-2-nitrobenzoate (128 g, 0.46 mol), 10% Pd/C (110 g, 103 mmol Pd), and methanol (1 L) was stirred overnight at room temperature under an atmosphere of H₂. The resulting mixture was filtered and concentrated to give 110 g of methyl 2-amino-3-(cyclopropylmethoxy)-4-methoxybenzoate as a white solid. MS (ESI): 252.1.

### Step 3

Sulfuric acid (1 mL) was added dropwise to a solution of methyl 2-amino-3-(cyclopropylmethoxy)-4-methoxybenzoate (98%, 187 g, 0.73 mol) and acetic anhydride (372 g, 3.65 mol). The reaction was warmed at 30° C. overnight, quenched with water/ice (1.5 L), and then stirred vigorously for 30 min. The resulting solids were collected by filtration and dried in an oven to give 140 g of methyl 2-acetamido-3-(cyclopropylmethoxy)-4-methoxybenzoate as a white solid. MS (ESI): 293.9.

### Step 4

A solution of methyl 2-acetamido-3-(cyclopropylmethoxy)-4-methoxybenzoate (93%, 55 g, 175 mmol) and tetrahydrofuran (500 mL) was added dropwise (internal temperature kept below −65° C.) to a solution of sodium bis(trimethylsilyl)amide (NaHMDS) (691 mL, 1M toluene, 691 mmol) at −78° C. under N₂. The cooling bath was removed, and the reaction was stirred overnight. The reaction was quenched with water/ice (1 L) and washed with diethyl ether (300 mL×2). The aqueous phase was adjusted to pH=3 with 6 N HCl. The resulting solids were collected by filtration, washed with water (500 mL×2), and dried in an oven to give 38.8 g of 8-(cyclopropylmethoxy)-4-hydroxy-7-methoxyquinolin-2(1H)-one as a yellow solid. MS (ESI): 262.0.

### Step 5

Ammonium acetate (620 g, 8.0 mol) was added to a solution of 8-(cyclopropylmethoxy)-4-hydroxy-7-methoxyquinolin-2(1H)-one (105 g, 0.40 mol), acetic acid (400 mL), and toluene (200 mL). The reaction was refluxed with a Dean-Stark apparatus for 2 h, and then the mixture was concentrated to remove the toluene. The reaction was then refluxed overnight. Water/ice (200 mL) was added, and with external cooling, the pH was adjusted to 14 using 40% KOH. The resulting solids were collected by filtration and then purified by silica gel chromatography (100:1→30:1; dichloromethane/methanol) to give 75 g of 4-amino-8-(cyclopropylmethoxy)-7-methoxyquinolin-2(1H)-one as a light yellow solid. ¹H NMR (400 MHz, DMSO-d₆): δ 9.20 (s, 1H), 7.59 (d, 1H), 6.88 (d, 1H), 6.53 (s, 2H), 5.29 (s, 1H), 3.86 (s, 3H), 3.81 (d, 2H), 1.22 (m, 1H), 0.44 (m, 2H), 0.22 (m, 2H); MS (ESI): 261.1.

### Intermediate 3

### 4-(3,5-Dichloropyridin-4-ylamino)-8-hydroxy-7-methoxyquinolin-2(1H)-one

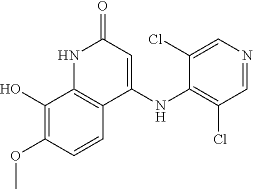 [SMILES: COc1ccc2c(Nc3c(Cl)cncc3Cl)cc(=O)[nH]c2c1O]

### Step 1

Sodium hydride (60%, 14 g, 350 mmol) was added (caution: H₂ gas evolution) to a solution of 4-amino-8-(cyclopropylmethoxy)-7-methoxyquinolin-2(1H)-one (18 g, 69 mmol) and DMSO (200 mL) under N₂. After 1 h at room temperature, 3,4,5-trichloropyridine (12.6 g, 69 mmol) was added portionwise with external cooling (internal temperature kept below 30° C.), and the reaction was warmed at 32° C. overnight. The reaction was quenched with water (40 mL) and adjusted to pH=6 with saturated potassium phosphate (KH₂PO₄). The resulting solids were collected by filtration, purified by silica gel chromatography (100:1; dichloromethane/methanol), and then triturated with ethyl acetate (300 mL) to give 22 g of 8-(cyclopropylmethoxy)-4-(3,5-dichloropyridin-4-ylamino)-7-methoxy-quinolin-2(1H)-one as a straw yellow solid. MS (ESI): 406.1.

### Step 2

A solution of 8-(cyclopropylmethoxy)-4-(3,5-dichloropyridin-4-ylamino)-7-methoxyquinolin-2(1H)-one (99%. 23.8 g, 57.9 mmol) and conc. HCl (150 mL) was warmed at 40° C. for 30 min and then water (0° C., 150 mL) was added. The resulting mixture was stirred vigorously at 0° C. for 1 h, filtered, and then washed with water (0° C., 100 mL). A mixture of the resulting solid and 0.5 M pH 7 phosphate buffer (300 mL) was stirred vigorously at 0° C. for 30 min, filtered, washed with water (0° C., 300 mL), and then dried in an oven to give 19.9 g of 4-(3,5-dichloropyridin-4-ylamino)-8-hydroxy-7-methoxyquinolin-2(1H)-one as a yellow solid. ¹H NMR (400 MHz, DMSO-d₆): δ 9.69 (s, 1H), 9.51 (s, 1H), 8.78 (s, 3H), 7.65 (d, 1H), 7.01 (d, 1H), 4.79 (s, 1H), 3.90 (s, 3H); MS (ESI): 352.2.

### Intermediate 4

### 8-(6-Chlorohexyloxy)-4-(3,5-dichloropyridin-4-ylamino)-7-methoxyquinolin-2(1H)-one

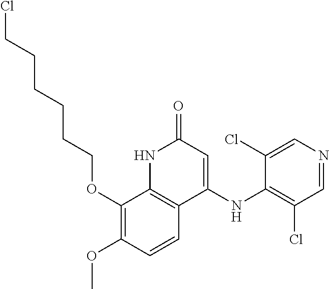 [SMILES: COc1ccc2c(Nc3c(Cl)cncc3Cl)cc(=O)[nH]c2c1OCCCCCCCl]

A mixture of 4-(3,5-dichloropyridin-4-ylamino)-8-hydroxy-7-methoxyquinolin-2(1H)-one (Intermediate 3) (19.9 g, 56.5 mmol), cesium carbonate (36.9 g, 113 mmol), and DMSO (190 mL) was heated at 30° C. for 15 minutes, and then 1-bromo-6-chlorohexane (12.4 g, 62.2 mmol) was added. The reaction was heated at 30° C. for 4.5 h, diluted with ethyl acetate (2 L), washed with 1M pH 7 phosphate buffer (2 L), and then washed with water (2 L×2). The organic layer was dried, filtered, concentrated, and then purified by silica gel chromatography (100% ethyl acetate) to give 19 g of 8-(6-chlorohexyloxy)-4-(3,5-dichloropyridin-4-ylamino)-7-methoxyquinolin-2(1H)-one as a yellow solid. ¹H NMR (400 MHz, DMSO-d₆): δ 10.01 (s, 1H), 8.87 (s, 1H), 8.79 (s, 2H), 7.90 (d, 1H), 7.06 (d, 1H), 4.80 (s, 1H), 3.98 (t, 2H), 3.92 (s, 3H), 3.65 (t, 2H), 1.77 (m, 4H), 1.43 (m, 4H); MS (ESI): 470.3.

### EXAMPLE 15

### 4-(3,5-Dichloropyridin-4-ylamino)-7-methoxy-8-(6-(4-methylpiperazin-1-yl)hexyloxy)quinolin-2(1H)-one

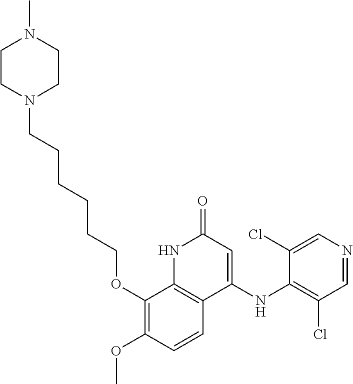 [SMILES: COc1ccc2c(Nc3c(Cl)cncc3Cl)cc(=O)[nH]c2c1OCCCCCCN1CCN(C)CC1]

1-Methylpiperazine (25.5 g, 254 mmol) was added to a solution of Intermediate 4 (10 g, 21 mmol) and DMSO (150 mL), and the solution was warmed to 30° C. After 48 h, the solution was diluted with water (500 mL) and extracted with ethyl acetate (500 mL×5). The combined extracts were dried, filtered, concentrated and then purified by silica gel chromatography (50:1; dichloromethane/methanol). Recrystallization of the product (7.9 g) with 10:1 methyl tert-butyl ether/dichloromethane (200 mL) gave 6.9 g of 4-(3,5-dichloropyridin-4-ylamino)-7-methoxy-8-(6-(4-methylpiperazin-1-yl)hexyloxy)quinolin-2(1H)-one as a white solid. ¹H NMR (400 MHz, DMSO-d₆): δ 9.94 (s, 1H), 8.83 (br, 1H), 8.77 (s, 2H), 7.88 (d, 1H), 7.03 (d, 1H), 4.78 (s, 1H), 3.98 (t, 2H), 3.79 (s, 3H), 2.28 (br, 8H), 2.21 (t, 2H), 2.11 (s, 3H), 1.74 (m, 2H), 1.39 (m, 4H), 1.28 (m, 2H); MS (ESI): 534.3.

### EXAMPLE 16

### 4-(3,5-Dichloropyridin-4-ylamino)-7-methoxy-8-(6-morpholinohexyloxy)quinolin-2(1H)-one

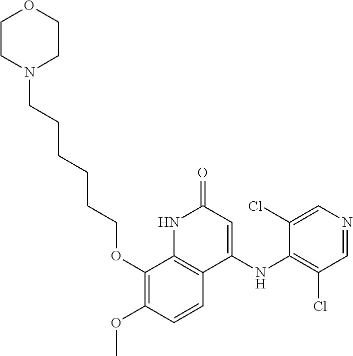 [SMILES: COc1ccc2c(Nc3c(Cl)cncc3Cl)cc(=O)[nH]c2c1OCCCCCCN1CCOCC1]

The title compound was prepared from 8-(6-chlorohexyloxy)-4-(3,5-dichloropyridin-4-ylamino)-7-methoxyquinolin-2(1H)-one (Intermediate 4) and morpholine following the procedure outlined in Example 15. ¹H NMR (400 MHz, DMSO-d₆): δ 9.97 (s, 1H), 8.83 (s, 1H), 8.77 (s, 2H), 7.88 (d, 1H), 7.04 (d, 1H), 4.79 (s, 1H), 3.97 (t, 2H), 3.79 (s, 3H), 3.52 (app t, 4H), 2.29 (br, 4H), 2.22 (t, 2H), 1.74 (m, 2H), 1.39 (m, 4H), 1.30 (m, 2H); MS (ESI): 521.3.

### EXAMPLE 17

### 4-(3,5-Dichloropyridin-4-ylamino)-8-(6-(dimethylamino)hexyloxy)-7-methoxyquinolin-2(1H)-one

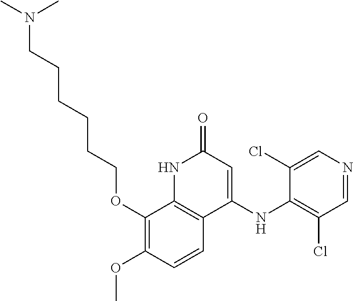 [SMILES: COc1ccc2c(Nc3c(Cl)cncc3Cl)cc(=O)[nH]c2c1OCCCCCCN(C)C]

### Step 1

8-(6-Bromohexyloxy)-4-(3,5-dichloropyridin-4-ylamino)-7-methoxyquinolin-2(1H)-one was prepared from 4-(3,5-dichloropyridin-4-ylamino)-8-hydroxy-7-methoxyquinolin-2(1H)-one (Intermediate 3) and 1,6-dibromohexane following the procedure outlined for Intermediate 4 (modifications: 1.3 equiv NaH and 2.5 equiv of dibromide used; reaction run at room temperature). MS (ESI): 514.0.

### Step 2

8-(6-Bromohexyloxy)-4-(3,5-dichloropyridin-4-ylamino)-7-methoxyquinolin-2(1H)-one (129 mg, 0.25 mmol) was added to a solution of dimethylamine (2.0 mL, 2M tetrahydrofuran, 4 mmol) and DMSO (0.5 mL) at room temperature. After 2.5 h, the reaction was concentrated to remove the excess amine and tetrahydrofuran. The resulting solution was purified by reverse-phase HPLC to give 4-(3,5-dichloropyridin-4-ylamino)-8-(6-(dimethylamino)hexyloxy)-7-methoxyquinolin-2(1H)-one. ¹H NMR (400 MHz, DMSO-d₆, HCl salt): δ 10.32 (s, 1H), 10.27 (br, 1H), 9.14 (s, 1H), 8.78 (s, 2H), 7.97 (d, 1H), 7.12 (d, 1H), 4.90 (s, 1H), 3.99 (t, 2H), 3.92 (s, 3H), 2.99 (m, 2H), 2.70 (d, 6H), 1.77 (m, 2H), 1.65 (m, 2H), 1.43 (m, 2H), 1.33 (m, 2H); MS (ESI): 479.0.

### EXAMPLE 18

### 4-(3,5-dichloropyridin-4-ylamino)-7-methoxy-8-(3-(4-methylpiperazin-1-yl)propoxy)quinolin-2(1H)-one

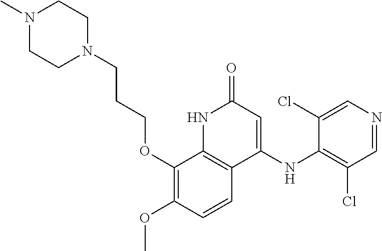 [SMILES: COc1ccc2c(Nc3c(Cl)cncc3Cl)cc(=O)[nH]c2c1OCCCN1CCN(C)CC1]

### Step 1

8-(3-Bromopropoxy)-4-(3,5-dichloropyridin-4-ylamino)-7-methoxyquinolin-2(1H)-one was prepared from 4-(3,5-dichloropyridin-4-ylamino)-8-hydroxy-7-methoxyquinolin-2(1H)-one (Intermediate 3) and 1,3-dibromopropane following the procedure outlined for Intermediate 4 (modifications: 1.0 equiv KHMDS and 5.0 equiv of dibromide used; reaction run at room temperature). MS (ESI): 472.1.

### Step 2

1-Methylpiperazine (0.5 mL, 4.5 mmol) was added to a solution of 8-(3-bromopropoxy)-4-(3,5-dichloropyridin-4-ylamino)-7-methoxyquinolin-2(1H)-one (150 mg, 0.32 mmol) and DMSO (2 mL) at room temperature. After 15 h, the reaction was placed under vacuum (˜0.1 Torr, 24 h) to remove the excess amine. The resulting solution was purified by reverse-phase HPLC to give 4-(3,5-dichloropyridin-4-ylamino)-7-methoxy-8-(3-(4-methylpiperazin-1-yl)propoxy)quinolin-2(1H)-one. ¹H NMR (400 MHz, DMSO-d₆, bis HCl salt): δ 12.01 (br, 2H), 10.80 (br, 1H), 9.21 (s, 1H), 8.79 (s, 2H), 8.01 (d, 1H), 7.15 (d, 1H), 4.94 (s, 1H), 4.04 (t, 2H), 3.95 (s, 3H), 3.90-3.60 (br m, 4H), 3.60-3.34 (br m, 6H), 2.82 (s, 3H), 2.25 (m, 2H); MS (ESI): 492.3.

### EXAMPLE 19

### 4-(3,5-dichloropyridin-4-ylamino)-7-methoxy-8-(3-morpholinopropoxy)quinolin-2(1H)-one

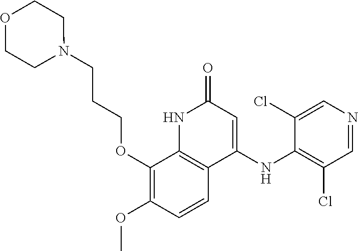 [SMILES: COc1ccc2c(Nc3c(Cl)cncc3Cl)cc(=O)[nH]c2c1OCCCN1CCOCC1]

The title compound was prepared from 8-(3-bromopropoxy)-4-(3,5-dichloropyridin-4-ylamino)-7-methoxyquinolin-2(1H)-one and morpholine following the procedure outlined in Example 18, Step 2. ¹H NMR (400 MHz, DMSO-d₆, HCl salt): δ 10.58 (br, 1H), 10.44 (s, 1H), 8.94 (s, 1H), 8.77 (s, 2H), 7.93 (d, 1H), 7.09 (d, 1H), 4.82 (s, 1H), 4.02 (t, 2H), 3.98 (m, 2H), 3.93 (s, 3H), 3.76 (t, 2H), 3.46 (d, 2H), 3.33 (m, 2H), 3.10 (m, 2H), 2.22 (m, 2H); MS (ESI): 479.3.

### EXAMPLE 20

### 4-(3,5-dichloropyridin-4-ylamino)-7-methoxy-8-(7-(4-methylpiperazin-1-yl)heptyloxy)quinolin-2(1H)-one

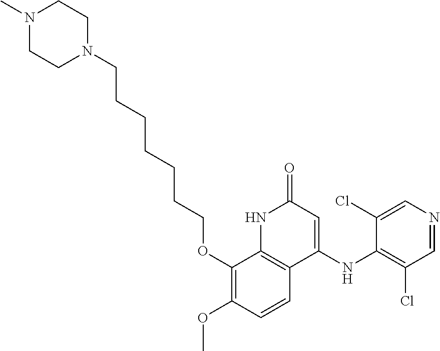 [SMILES: COc1ccc2c(Nc3c(Cl)cncc3Cl)cc(=O)[nH]c2c1OCCCCCCCN1CCN(C)CC1]

The title compound was prepared from 4-(3,5-dichloropyridin-4-ylamino)-8-hydroxy-7-methoxyquinolin-2(1H)-one (Intermediate 3), 1,7-dibromoheptane, and 1-methylpiperazine following the procedures outlined for Intermediate 4 (modifications: NMP (N-methylpyrrolidone) used as solvent and 5.0 equiv of dibromide used) and Example 18, Step 2. ¹H NMR (400 MHz, DMSO-d₆, bis HCl salt): δ 11.87 (br, 2H), 10.46 (s, 1H), 9.22 (s, 1H), 8.81 (s, 2H), 8.01 (d, 1H), 7.16 (d, 1H), 4.95 (s, 1H), 4.01 (t, 2H), 3.94 (s, 3H), 3.84-3.20 (br m, 8H), 3.11 (m, 2H), 2.82 (s, 3H), 1.84-1.68 (m, 4H), 1.48-1.30 (m, 6H); MS (ESI): 548.3.

### EXAMPLE 21

### 4-(3,5-dichloropyridin-4-ylamino)-7-methoxy-8-(8-(4-methylpiperazin-1-yl)octyloxy)quinolin-2(1H)-one

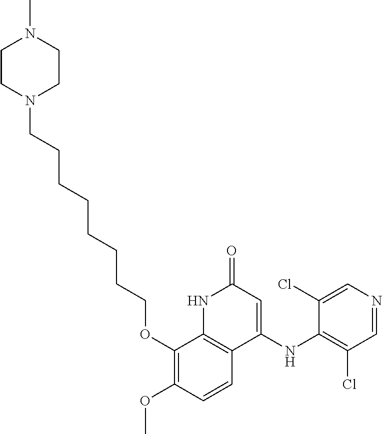 [SMILES: COc1ccc2c(Nc3c(Cl)cncc3Cl)cc(=O)[nH]c2c1OCCCCCCCCN1CCN(C)CC1]

The title compound was prepared from 4-(3,5-dichloropyridin-4-ylamino)-8-hydroxy-7-methoxyquinolin-2(1H)-one (Intermediate 3), 1,8-dibromooctane, and 1-methylpiperazine following the procedures outlined for Intermediate 4 (modifications: NMP used as solvent and 5.0 equiv of dibromide used) and Example 18, Step 2. ¹H NMR (400 MHz, DMSO-d₆, bis HCl salt): δ 11.89 (br, 2H), 10.49 (s, 1H), 9.24 (s, 1H), 8.81 (s, 2H), 8.01 (d, 1H), 7.16 (d, 1H), 4.97 (s, 1H), 4.01 (t, 2H), 3.94 (s, 3H), 3.82-3.20 (br m, 8H), 3.10 (m, 2H), 2.81 (s, 3H), 1.84-1.66 (m, 4H), 1.46-1.26 (m, 8H); MS (ESI): 562.3.

### EXAMPLE 22

### 4-(3,5-Dichloropyridin-4-ylamino)-7-methoxy-8-(6-(piperazin-1-yl)hexyloxy)quinolin-2(1H)-one

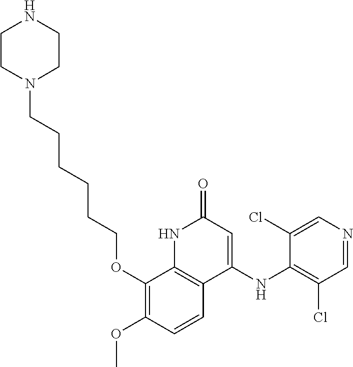 [SMILES: COc1ccc2c(Nc3c(Cl)cncc3Cl)cc(=O)[nH]c2c1OCCCCCCN1CCNCC1]

The title compound was prepared from 8-(6-chlorohexyloxy)-4-(3,5-dichloropyridin-4-ylamino)-7-methoxyquinolin-2(1H)-one (Intermediate 4) and piperazine following the procedure outlined in Example 15 (modifications: 40° C., 8 h, purified by reverse-phase HPLC). ¹H NMR (400 MHz, DMSO-d₆, bis HCl salt): δ 11.79 (br, 1H), 10.51 (s, 1H), 9.81 (br, 1H), 9.73 (br, 1H), 9.25 (s, 1H), 8.82 (s, 2H), 8.02 (d, 1H), 7.17 (d, 1H), 4.97 (s, 1H), 4.01 (t, 2H), 3.95 (s, 3H), 3.68 (br d, 2H), 3.58-3.35 (br m, 4H), 3.26 (br, 2H), 3.13 (m, 2H), 1.84-1.70 (m, 4H), 1.50-1.32 (m, 4H); MS (ESI): 520.4.

### EXAMPLE 23

### 4-(3,5-dichloropyridin-4-ylamino)-8-(6-(4-ethylpiperazin-1-yl)hexyloxy)-7-methoxyquinolin-2(1H)-one

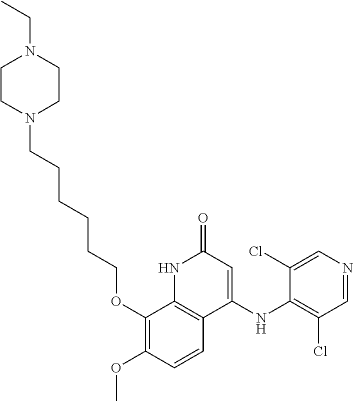 [SMILES: CCN1CCN(CCCCCCOc2c(OC)ccc3c(Nc4c(Cl)cncc4Cl)cc(=O)[nH]c23)CC1]

The title compound was prepared from 8-(6-chlorohexyloxy)-4-(3,5-dichloropyridin-4-ylamino)-7-methoxyquinolin-2(1H)-one (Intermediate 4) and 1-ethylpiperazine following the procedure outlined in Example 18, Step 2 (modifications: 40° C., 24 h). ¹H NMR (400 MHz, DMSO-d₆, bis HCl salt): δ 12.00-11.60 (br, 2H), 10.46 (s, 1H), 9.21 (s, 1H), 8.81 (s, 2H), 8.01 (d, 1H), 7.16 (d, 1H), 4.95 (s, 1H), 4.01 (t, 2H), 3.95 (s, 3H), 3.84-3.35 (br m, 8H), 3.35-3.10 (br m, 4H), 1.84-1.70 (m, 4H), 1.52-1.32 (m, 4H), 1.27 (t, 3H); MS (ESI): 548.5.

### EXAMPLE 24

### 4-(3,5-dichloropyridin-4-ylamino)-8-(6-((2-(dimethylamino)ethyl)(methyl)amino)hexyloxy)-7-methoxyquinolin-2(1H)-one

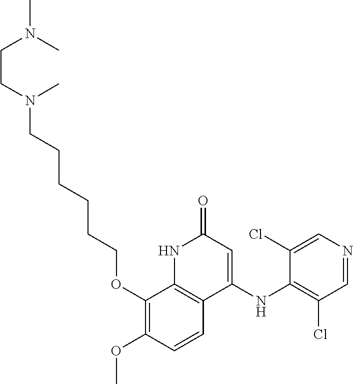 [SMILES: COc1ccc2c(Nc3c(Cl)cncc3Cl)cc(=O)[nH]c2c1OCCCCCCN(C)CCN(C)C]

The title compound was prepared from 8-(6-chlorohexyloxy)-4-(3,5-dichloropyridin-4-ylamino)-7-methoxyquinolin-2(1H)-one (Intermediate 4) and N¹,N¹,N²-trimethylethane-1,2-diamine following the procedure outlined in Example 18, Step 2 (modifications: 40° C., 24 h then 60° C., 4 h). ¹H NMR (400 MHz, DMSO-d₆, bis HCl salt): δ 10.91 (br, 2H), 10.26 (s, 1H), 9.11 (s, 1H), 8.81 (s, 2H), 7.98 (d, 1H), 7.14 (d, 1H), 4.90 (s, 1H), 4.01 (t, 2H), 3.94 (s, 3H), 3.63-3.42 (br m, 4H), 3.25-3.00 (br m, 2H), 2.90-2.78 (m, 9H), 1.85-1.69 (m, 4H), 1.52-1.32 (m, 4H); MS (ESI): 536.4.

### EXAMPLE 25

### 8-(6-(1,4-Diazepan-1-yl)hexyloxy)-4-(3,5-dichloropyridin-4-ylamino)-7-methoxyquinolin-2(1H)-one

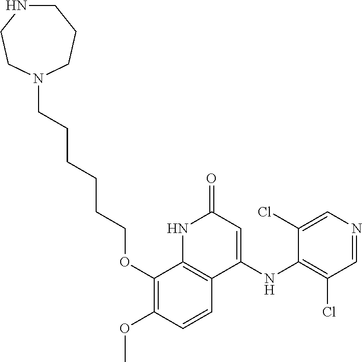 [SMILES: COc1ccc2c(Nc3c(Cl)cncc3Cl)cc(=O)[nH]c2c1OCCCCCCN1CCCNCC1]

The title compound was prepared from 8-(6-chlorohexyloxy)-4-(3,5-dichloropyridin-4-ylamino)-7-methoxyquinolin-2(1H)-one (Intermediate 4) and 1,4-diazepane following the procedure outlined in Example 18, Step 2 (modifications: 40° C., 8 h). ¹H NMR (400 MHz, DMSO-d₆): δ 8.68 (s, 2H), 7.85 (d, 1H), 7.00 (d, 1H), 4.69 (s, 1H), 3.95 (t, 2H), 3.88 (s, 3H), 2.79 (t, 2H), 2.74 (m, 2H), 2.60-2.52 (m, 4H), 2.40 (t, 2H), 1.74 (m, 2H), 1.63 (m, 2H), 1.39 (m, 4H), 1.30 (m, 2H) [NH's not seen]; MS (ESI): 534.3.

### EXAMPLE 26

### 4-(3,5-Dichloropyridin-4-ylamino)-7-methoxy-8-(6-(4-methyl-1,4-diazepan-1-yl)hexyloxy)quinolin-2(1H)-one

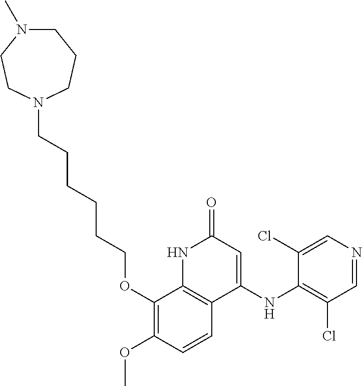 [SMILES: COc1ccc2c(Nc3c(Cl)cncc3Cl)cc(=O)[nH]c2c1OCCCCCCN1CCCN(C)CC1]

The title compound was prepared from 8-(6-chlorohexyloxy)-4-(3,5-dichloropyridin-4-ylamino)-7-methoxyquinolin-2(1H)-one (Intermediate 4) and 1-methyl-1,4-diazepane following the procedure outlined in Example 18, Step 2 (modifications: 40° C., 24 h). ¹H NMR (400 MHz, DMSO-d₆): δ 9.93 (s, 1H), 8.82 (br, 1H), 8.75 (s, 2H), 7.87 (d, 1H), 7.04 (d, 1H), 4.76 (s, 1H), 3.95 (t, 2H), 3.89 (s, 3H), 2.62-2.42 (m, 8H), 2.37 (t, 2H), 2.21 (s, 3H), 1.74 (m, 2H), 1.66 (m, 2H), 1.38 (m, 4H), 1.29 (m, 2H); MS (ESI): 548.3.

### EXAMPLE 27

### 4-(3,5-Dichloropyridin-4-ylamino)-8-(6-((3-(dimethylamino)propyl)(methyl)amino)hexyloxy)-7-methoxyquinolin-2(1H)-one

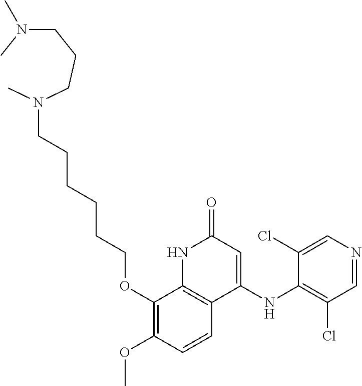 [SMILES: COc1ccc2c(Nc3c(Cl)cncc3Cl)cc(=O)[nH]c2c1OCCCCCCN(C)CCCN(C)C]

The title compound was prepared from 8-(6-chlorohexyloxy)-4-(3,5-dichloropyridin-4-ylamino)-7-methoxyquinolin-2(1H)-one (Intermediate 4) and N¹,N¹,N³-trimethylpropane-1,3-diamine following the procedure outlined in Example 18, Step 2 (modifications: 40° C., 24 h then 60° C., 4 h). ¹H NMR (400 MHz, DMSO-d₆, bis HCl salt): δ 10.78-10.62 (br, 2H), 10.33 (s, 1H), 9.17 (s, 1H), 8.81 (s, 2H), 8.00 (d, 1H), 7.15 (d, 1H), 4.93 (s, 1H), 4.01 (t, 2H), 3.94 (s, 3H), 3.25-2.95 (m, 6H), 2.76-2.70 (m, 9H), 2.13 (m, 2H), 1.80 (m, 2H), 1.73 (m, 2H), 1.47 (m, 2H), 1.37 (m, 2H); MS (ESI): 550.5.

### EXAMPLE 28

### 8-(6-Aminohexyloxy)-4-(3,5-dichloropyridin-4-ylamino)-7-methoxyquinolin-2(1H)-one

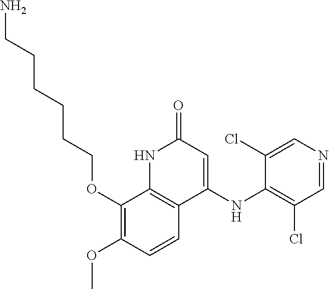 [SMILES: COc1ccc2c(Nc3c(Cl)cncc3Cl)cc(=O)[nH]c2c1OCCCCCCN]

The title compound was prepared from 8-(6-chlorohexyloxy)-4-(3,5-dichloropyridin-4-ylamino)-7-methoxyquinolin-2(1H)-one (Intermediate 4) and 28% ammonium hydroxide following the procedure outlined in Example 18, Step 2 (modifications: 40° C., 24 h, then second aliquot of NH₄OH added, then 60° C., 24 h). ¹H NMR (400 MHz, DMSO-d₆, HCl salt): δ 10.57 (s, 1H), 9.30 (s, 1H), 8.82 (s, 2H), 8.03 (d, 1H), 7.94 (br, 3H), 7.18 (d, 1H), 4.99 (s, 1H), 4.01 (t, 2H), 3.95 (s, 3H), 2.77 (m, 2H), 1.78 (m, 2H), 1.58 (m, 2H), 1.40 (m, 4H); MS (ESI): 451.3.

### EXAMPLE 29

### 4-(3,5-Dichloropyridin-4-ylamino)-8-(6-(2-hydroxyethylamino)hexyloxy)-7-methoxyquinolin-2(1H)-one

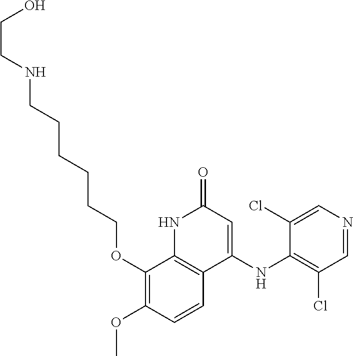 [SMILES: COc1ccc2c(Nc3c(Cl)cncc3Cl)cc(=O)[nH]c2c1OCCCCCCNCCO]

The title compound was prepared from 8-(6-chlorohexyloxy)-4-(3,5-dichloropyridin-4-ylamino)-7-methoxyquinolin-2(1H)-one (Intermediate 4) and 2-aminoethanol following the procedure outlined in Example 18, Step 2 (modifications: 40° C., 24 h). ¹H NMR (400 MHz, DMSO-d₆): δ 8.70 (s, 2H), 7.86 (d, 1H), 7.01 (d, 1H), 4.70 (s, 1H), 4.52 (br, 1H), 3.95 (t, 2H), 3.89 (s, 3H), 3.43 (t, 2H), 2.58 (t, 2H), 2.53 (m, 2H), 1.74 (m, 2H), 1.48-1.28 (m, 6H) [NH's not seen]; MS (ESI): 495.3.

### EXAMPLE 30

### 4-(3,5-Dichloropyridin-4-ylamino)-8-(6-((2-hydroxyethyl)(methyl)amino)hexyloxy)-7-methoxyquinolin-2(1H)-one

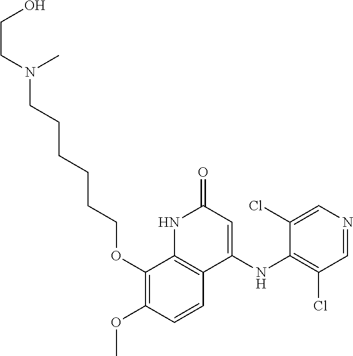 [SMILES: COc1ccc2c(Nc3c(Cl)cncc3Cl)cc(=O)[nH]c2c1OCCCCCCN(C)CCO]

The title compound was prepared from 8-(6-chlorohexyloxy)-4-(3,5-dichloropyridin-4-ylamino)-7-methoxyquinolin-2(1H)-one (Intermediate 4) and 2-(methylamino)ethanol following the procedure outlined in Example 18, Step 2 (modifications: 40° C., 24 h). ¹H NMR (400 MHz, DMSO-d₆): δ 9.94 (s, 1H), 8.83 (s, 1H), 8.75 (s, 2H), 7.87 (d, 1H), 7.04 (d, 1H), 4.76 (s, 1H), 4.32 (br, 1H), 3.95 (t, 2H), 3.89 (s, 3H), 3.43 (m, 2H), 2.36 (t, 2H), 2.29 (t, 2H), 2.13 (s, 3H), 1.74 (m, 2H), 1.39 (m, 4H), 1.29 (m, 2H); MS (ESI): 509.3.

### EXAMPLE 31

### 8-(6-(Bis(2-hydroxyethyl)amino)hexyloxy)-4-(3,5-dichloropyridin-4-ylamino)-7-methoxyquinolin-2(1H)-one

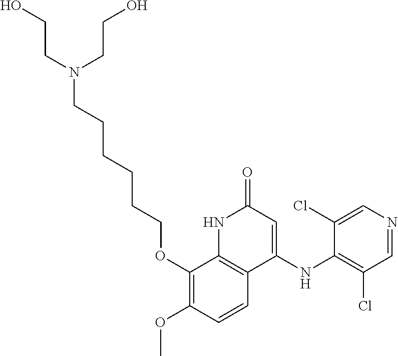 [SMILES: COc1ccc2c(Nc3c(Cl)cncc3Cl)cc(=O)[nH]c2c1OCCCCCCN(CCO)CCO]

The title compound was prepared from 8-(6-chlorohexyloxy)-4-(3,5-dichloropyridin-4-ylamino)-7-methoxyquinolin-2(1H)-one (Intermediate 4) and diethanolamine following the procedure outlined in Example 18, Step 2 (modifications: 40° C., 24 h then 60° C., 24 h). ¹H NMR (400 MHz, DMSO-d₆): δ 9.95 (s, 1H), 8.83 (s, 1H), 8.76 (s, 2H), 7.87 (d, 1H), 7.04 (d, 1H), 4.77 (s, 1H), 4.30 (br, 2H), 3.95 (t, 2H), 3.90 (s, 3H), 3.39 (m, 4H), 2.56-2.36 (m, 6H), 1.74 (m, 2H), 1.38 (m, 4H), 1.28 (m, 2H); MS (ESI): 539.3.

### EXAMPLE 32

### 4-(3,5-Dichloropyridin-4-ylamino)-8-(6-(3-hydroxypropylamino)hexyloxy)-7-methoxyquinolin-2(1H)-one

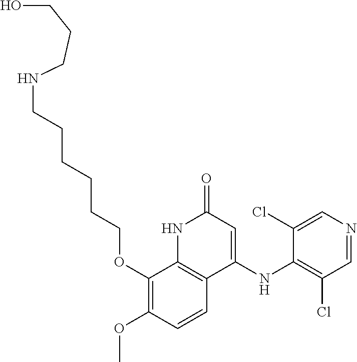 [SMILES: COc1ccc2c(Nc3c(Cl)cncc3Cl)cc(=O)[nH]c2c1OCCCCCCNCCCO]

The title compound was prepared from 8-(6-chlorohexyloxy)-4-(3,5-dichloropyridin-4-ylamino)-7-methoxyquinolin-2(1H)-one (Intermediate 4) and 3-aminopropan-1-ol following the procedure outlined in Example 18, Step 2 (modifications: 40° C., 24 h). ¹H NMR (400 MHz, DMSO-d₆): δ 8.67 (s, 2H), 7.85 (d, 1H), 7.00 (d, 1H), 4.68 (s, 1H), 3.95 (t, 2H), 3.88 (s, 3H), 3.43 (t, 2H), 2.59 (t, 2H), 2.52 (m, 2H), 1.74 (m, 2H), 1.54 (m, 2H), 1.48-1.28 (m, 6H) [NH's & OH not seen]; MS (ESI): 509.3.

### EXAMPLE 33

### 4-(3,5-Dichloropyridin-4-ylamino)-8-(6-((3-hydroxypropyl)(methyl)amino)hexyloxy)-7-methoxyquinolin-2(1H)-one

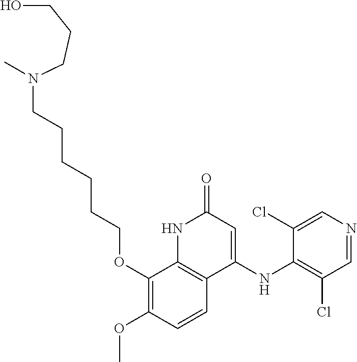 [SMILES: COc1ccc2c(Nc3c(Cl)cncc3Cl)cc(=O)[nH]c2c1OCCCCCCN(C)CCCO]

The title compound was prepared from 8-(6-chlorohexyloxy)-4-(3,5-dichloropyridin-4-ylamino)-7-methoxyquinolin-2(1H)-one (Intermediate 4) and 3-(methylamino)propan-1-ol following the procedure outlined in Example 18, Step 2 (modifications: 40° C., 24 h). ¹H NMR (400 MHz, DMSO-d₆): δ 9.95 (s, 1H), 8.83 (s, 1H), 8.75 (s, 2H), 7.87 (d, 1H), 7.04 (d, 1H), 4.76 (s, 1H), 4.52 (br, 1H), 3.95 (t, 2H), 3.89 (s, 3H), 3.40 (t, 2H), 2.31 (t, 2H), 2.25 (t, 2H), 2.09 (s, 3H), 1.74 (m, 2H), 1.52 (m, 2H), 1.39 (m, 4H), 1.29 (m, 2H); MS (ESI): 523.3.

### Intermediate 5

### 4-(3,5-Dimethylpyridin-4-ylamino)-8-hydroxy-7-methoxyquinolin-2(1H)-one

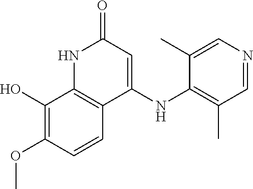 [SMILES: COc1ccc2c(Nc3c(C)cncc3C)cc(=O)[nH]c2c1O]

### Step 1

A mixture of 4-amino-8-(cyclopropylmethoxy)-7-methoxyquinolin-2(1H)-one (500 mg, 1.92 mmol), 4-bromo-3,5-dimethylpyridine (350 mg, 1.88 mmol), Pd₂ (dba)₃ (88 mg, 0.19 mmol Pd), 2-dicyclohexylphosphino-2′,4′,6′-triisopropylbiphenyl (XPhos) (181 mg, 0.38 mmol), sodium tert-butoxide (370 mg, 3.85 mmol), and toluene (20 mL) was heated at 110° C. for 3 h under N₂ and then allowed to cool to room temperature. The reaction was filtered and washed with 5% methanol in dichloromethane (500 mL×3). The filtrate was concentrated and purified by silica gel chromatography (40:1; ethyl acetate/methanol) and then reverse-phase HPLC to give 0.16 g of 8-(cyclopropylmethoxy)-4-(3,5-dimethylpyridin-4-ylamino)-7-methoxyquinolin-2(1H)-one as white solid. MS (ESI): 366.2

### Step 2

A solution of 8-(cyclopropylmethoxy)-4-(3,5-dimethylpyridin-4-ylamino)-7-methoxyquinolin-2(1H)-one (1.1 g, 3.0 mmol) and conc. HCl (6 mL) was maintained at room temperature for 3 h and then cooled to 0° C. This cold solution was poured into a solution of 0.5 M pH 7 phosphate buffer (75 mL) and 10N NaOH (7.5 mL) at 0° C. The resulting mixture was stirred vigorously at 0° C. for 15 min, filtered, washed with water (0° C., 75 mL), and then dried under vacuum to give 0.92 g of 4-(3,5-dimethylpyridin-4-ylamino)-8-hydroxy-7-methoxyquinolin-2(1H)-one. ¹H NMR (400 MHz, DMSO-d₆): δ 9.43 (s, 1H), 9.37 (s, 1H), 8.40 (s, 2H), 8.35 (s, 1H), 7.65 (d, 1H), 6.98 (d, 1H), 4.50 (s, 1H), 3.88 (s, 3H), 2.12 (s, 6H); MS (ESI): 312.6.

### Intermediate 6

### 8-(6-Chlorohexyloxy)-4-(3,5-dimethylpyridin-4-ylamino)-7-methoxyquinolin-2(1H)-one

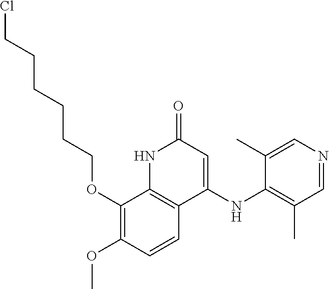 [SMILES: COc1ccc2c(Nc3c(C)cncc3C)cc(=O)[nH]c2c1OCCCCCCCl]

A mixture of 4-(3,5-dimethylpyridin-4-ylamino)-8-hydroxy-7-methoxyquinolin-2(1H)-one (1.46 g, 4.7 mmol), cesium carbonate (1.83 g, 5.6 mmol), and DMSO (15 mL) was stirred at room temperature for 15 min, and then 1-bromo-6-chlorohexane (0.83 mL, 5.6 mmol) was added. After 45 min, the reaction was diluted with ethyl acetate, washed with water (×3), dried, filtered, concentrated, and then triturated with hexane/diethyl ether to remove the excess 1-bromo-6-chlorohexane. The solid residue was purified by silica gel chromatography (49:1→23:2; dichloromethane/methanol) to give 8-(6-chlorohexyloxy)-4-(3,5-dimethylpyridin-4-ylamino)-7-methoxyquinolin-2(1H)-one. ¹H NMR (400 MHz, CDCl₃): δ 8.59 (s, 1H), 8.38 (s, 2H), 7.42 (d, 1H), 6.90 (d, 1H), 6.05 (s, 1H), 5.13 (d, 1H), 4.12 (t, 2H), 3.97 (s, 3H), 3.56 (t, 2H), 2.21 (s, 6H), 1.82 (m, 4H), 1.52 (m, 4H); MS (ESI): 430.3.

### EXAMPLE 34

### 4-(3,5-Dimethylpyridin-4-ylamino)-7-methoxy-8-(6-(4-methylpiperazin-1-yl)hexyloxy)quinolin-2(1H)-one

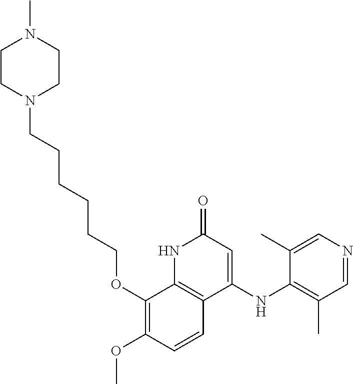 [SMILES: COc1ccc2c(Nc3c(C)cncc3C)cc(=O)[nH]c2c1OCCCCCCN1CCN(C)CC1]

The title compound was prepared from 8-(6-chlorohexyloxy)-4-(3,5-dimethylpyridin-4-ylamino)-7-methoxyquinolin-2(1H)-one (Intermediate 6) and 1-methylpiperazine following the procedure outlined in Example 15 (modifications: 25° C., overnight). ¹H NMR (400 MHz, DMSO-d₆, bis HCl salt): δ 10.09 (s, 1H), 8.82 (s, 1H), 8.59 (s, 2H), 7.88 (d, 1H), 7.06 (d, 1H), 4.99 (s, 1H), 3.98 (t, 2H), 3.91 (s, 3H), 3.75-2.90 (br, 10H), 2.77 (s, 3H), 2.22 (s, 6H), 1.77 (m, 2H), 1.69 (m, 2H), 1.44 (m, 2H), 1.36 (m, 2H); MS (ESI): 494.4.

### EXAMPLE 35

### 4-(3,5-Dimethylpyridin-4-ylamino)-7-methoxy-8-(6-morpholinohexyloxy)quinolin-2(1H)-one

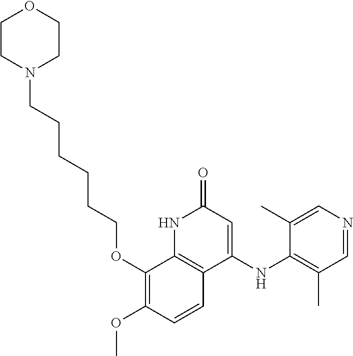 [SMILES: COc1ccc2c(Nc3c(C)cncc3C)cc(=O)[nH]c2c1OCCCCCCN1CCOCC1]

The title compound was prepared from 8-(6-chlorohexyloxy)-4-(3,5-dimethylpyridin-4-ylamino)-7-methoxyquinolin-2(1H)-one (Intermediate 6) and morpholine following the procedure outlined in Example 15 (modifications: 25° C., 16 h; then 40° C., 24 h). ¹H NMR (400 MHz, DMSO-d₆): δ 9.62 (s, 1H), 8.40 (s, 1H), 8.39 (s, 2H), 7.88 (d, 1H), 7.01 (d, 1H), 4.51 (s, 1H), 3.96 (t, 2H), 3.89 (s, 3H), 3.53 (m, 4H), 2.29 (br, 4H), 2.22 (t, 2H), 2.13 (s, 6H), 1.74 (m, 2H), 1.39 (m, 4H), 1.31 (m, 2H); MS (ESI): 481.4.

### EXAMPLE 36

### 4-(3,5-Dimethylpyridin-4-ylamino)-7-methoxy-8-(6-(piperidin-1-yl)hexyloxy)quinolin-2(1H)-one

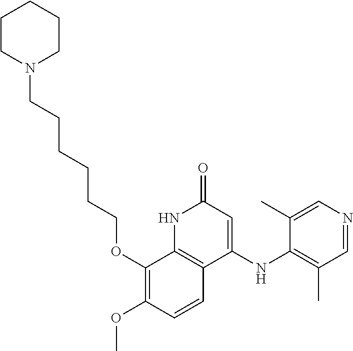 [SMILES: COc1ccc2c(Nc3c(C)cncc3C)cc(=O)[nH]c2c1OCCCCCCN1CCCCC1]

The title compound was prepared from 8-(6-chlorohexyloxy)-4-(3,5-dimethylpyridin-4-ylamino)-7-methoxyquinolin-2(1H)-one (Intermediate 6) and piperidine following the procedure outlined in Example 15 (modifications: 40° C., 18 h). ¹H NMR (400 MHz, DMSO-d₆): δ 9.64 (s, 1H), 8.39 (s, 3H), 7.88 (d, 1H), 7.01 (d, 1H), 4.51 (s, 1H), 3.95 (t, 2H), 3.89 (s, 3H), 2.25 (br, 4H), 2.17 (t, 2H), 2.13 (s, 6H), 1.74 (m, 2H), 1.49-1.23 (m, 12H); MS (ESI): 479.4.

### EXAMPLE 37

### 4-(3,5-Dimethylpyridin-4-ylamino)-7-methoxy-8-(6-(pyrrolidin-1-yl)hexyloxy)quinolin-2(1H)-one

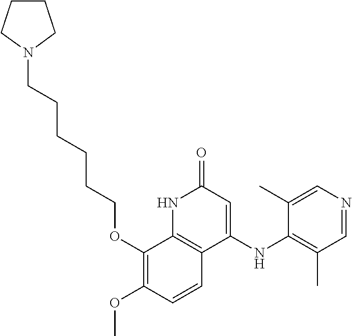 [SMILES: COc1ccc2c(Nc3c(C)cncc3C)cc(=O)[nH]c2c1OCCCCCCN1CCCC1]

The title compound was prepared from 8-(6-chlorohexyloxy)-4-(3,5-dimethylpyridin-4-ylamino)-7-methoxyquinolin-2(1H)-one (Intermediate 6) and pyrrolidine following the procedure outlined in Example 15 (modifications: 40° C., 18 h). ¹H NMR (400 MHz, DMSO-d₆; HCl salt): δ 10.51 (br, 1H), 9.73 (s, 1H), 8.58 (s, 1H), 8.44 (s, 2H), 7.92 (d, 1H), 7.03 (d, 1H), 4.63 (s, 1H), 3.97 (t, 2H), 3.90 (s, 3H), 3.46 (m, 2H), 3.06 (m, 2H), 2.93 (m, 2H), 2.15 (s, 6H), 1.95 (m, 2H), 1.84 (m, 2H), 1.76 (m, 2H), 1.67 (m, 2H), 1.43 (m, 2H), 1.35 (m, 2H); MS (ESI): 465.4.

### EXAMPLE 38

### 8-(6-(Dimethylamino)hexyloxy)-4-(3,5-dimethylpyridin-4-ylamino)-7-methoxyquinolin-2(1H)-one

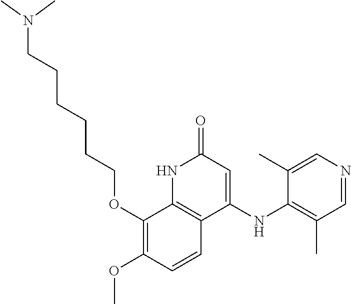 [SMILES: COc1ccc2c(Nc3c(C)cncc3C)cc(=O)[nH]c2c1OCCCCCCN(C)C]

### EXAMPLE 39

### 4-(3,5-Dimethylpyridin-4-ylamino)-8-(6-((3-hydroxypropyl)(methyl)amino)hexyloxy)-7-methoxyquinolin-2(1H)-one

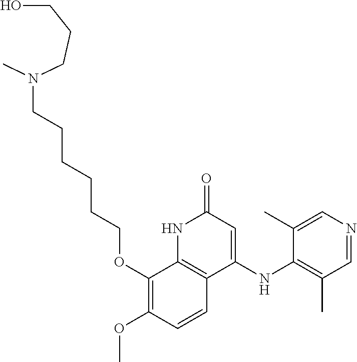 [SMILES: COc1ccc2c(Nc3c(C)cncc3C)cc(=O)[nH]c2c1OCCCCCCN(C)CCCO]

The title compound was prepared from 8-(6-chlorohexyloxy)-4-(3,5-dimethylpyridin-4-ylamino)-7-methoxyquinolin-2(1H)-one (Intermediate 6) and 3-(methylamino)propan-1-ol following the procedure outlined in Example 18, Step 2 (modifications: 22° C., 60 h). ¹H NMR (300 MHz, DMSO-d₆):): δ: 9.64 (s, 1H), 8.41 (s, 3H), 7.90 (d, J=9 Hz, 1H), 7.04 (d, J=9 Hz, 1H), 4.54 (s, 2H), 3.98 (t, J=6.9 Hz, 2H), 3.43 (t, J=6 Hz, 3H), 2.37-2.25 (4H, m), 2.14 (d, J=9.3 Hz, 8H), 1.76 (t, J=6.9 Hz, 2H), 1.37 (m, 6H). MS (ESI): 483.

### EXAMPLE 40

### 4-(3,5-Dimethylpyridin-4-ylamino)-8-(6-((2-hydroxyethyl)(methyl)amino)hexyloxy)-7-methoxyquinolin-2(1H)-one

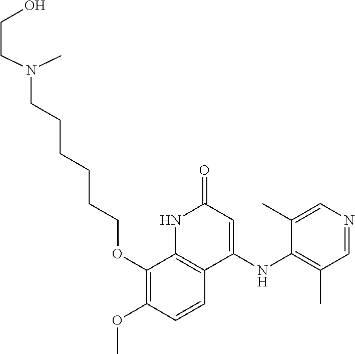 [SMILES: COc1ccc2c(Nc3c(C)cncc3C)cc(=O)[nH]c2c1OCCCCCCN(C)CCO]

The title compound was prepared from 8-(6-chlorohexyloxy)-4-(3,5-dimethylpyridin-4-ylamino)-7-methoxyquinolin-2(1H)-one (Intermediate 6) and 2-(methylamino)ethanol following the procedure outlined in Example 18, Step 2 (modifications: 22° C., 60 h). ¹H NMR (300 MHz, DMSO-d₆):): δ: 9.67 (s, 1H), 8.42 (s, 3H), 7.91 (d, J=9 Hz, 1H), 7.04 (s, J=7 Hz, 1H), 4.55 (s, 1H), 4.31 (s, 1H), 3.98 (t, J=6.6 Hz, 5H), 3.46 (s, 2H), 2.40-2.28 (m, 4H), 2.15 (s, 9H), 1.75 (m, 2H), 1.35 (m, 6H). MS (ESI) 469.

| Example | Name |
|---|---|
| 41 | 4-(3,5-dichloropyridin-4-ylamino)-8-(4-hydroxybutoxy)-7- |
| | methoxyquinolin-2(1H)-one |
| 42 | 4-(3,5-dichloropyridin-4-ylamino)-8-(5-hydroxypentyloxy)-7- |
| | methoxyquinolin-2(1H)-one |
| 43 | 4-(3,5-dichloropyridin-4-ylamino)-8-(6-hydroxyhexyloxy)-7- |
| | methoxyquinolin-2(1H)-one |
| 44 | 8-(4-(dimethylamino)butoxy)-4-(3,5-dimethylpyridin-4- |
| | ylamino)-7-methoxyquinolin-2(1H)-one |
| 45 | 8-(5-(dimethylamino)pentyloxy)-4-(3,5-dimethylpyridin-4- |
| | ylamino)-7-methoxyquinolin-2(1H)-one |
| 46 | 4-(3,5-dichloropyridin-4-ylamino)-7-methoxy-8-(4-(4- |
| | methylpiperazin-1-yl)butoxy)quinolin-2(1H)-one |
| 47 | 4-(3,5-dichloropyridin-4-ylamino)-7-methoxy-8-(5-(4- |
| | methylpiperazin-1-yl)pentyloxy)quinolin-2(1H)-one |
| 48 | 4-(3,5-dichloropyridin-4-ylamino)-7-methoxy-8-(4- |
| | morpholinobutoxy)quinolin-2(1H)-one |
| 49 | 4-(3,5-dichloropyridin-4-ylamino)-7-methoxy-8-(5- |
| | morpholinopentyloxy)quinolin-2(1H)-one |
| 50 | 2-(4-(3,5-dichloropyridin-4-ylamino)-7-methoxy-2-oxo-1,2- |
| | dihydroquinolin-8-yloxy)acetamide |
| 51 | 4-(4-(3,5-dichloropyridin-4-ylamino)-7-methoxy-2-oxo-1,2- |
| | dihydroquinolin-8-yloxy)butanoic acid |
| 52 | 5-(4-(3,5-dichloropyridin-4-ylamino)-7-methoxy-2-oxo-1,2- |
| | dihydroquinolin-8-yloxy)pentanoic acid |
| 53 | 7-(4-(3,5-dichloropyridin-4-ylamino)-7-methoxy-2-oxo-1,2- |
| | dihydroquinolin-8-yloxy)heptanoic acid |
| 54 | 8-(4-(3,5-dichloropyridin-4-ylamino)-7-methoxy-2-oxo-1,2- |
| | dihydroquinolin-8-yloxy)octanoic acid |
| 55 | 9-(4-(3,5-dichloropyridin-4-ylamino)-7-methoxy-2-oxo-1,2- |
| | dihydroquinolin-8-yloxy)nonanoic acid |
| 56 | 4-(4-(3,5-dichloropyridin-4-ylamino)-7-methoxy-2-oxo-1,2- |
| | dihydroquinolin-8-yloxy)butanamide |
| 57 | 5-(4-(3,5-dichloropyridin-4-ylamino)-7-methoxy-2-oxo-1,2- |
| | dihydroquinolin-8-yloxy)pentanamide |
| 58 | 6-(4-(3,5-dichloropyridin-4-ylamino)-7-methoxy-2-oxo-1,2- |
| | dihydroquinolin-8-yloxy)hexanamide |
| 59 | 7-(4-(3,5-dichloropyridin-4-ylamino)-7-methoxy-2-oxo-1,2- |
| | dihydroquinolin-8-yloxy)heptanamide |
| 60 | 8-(4-(3,5-dichloropyridin-4-ylamino)-7-methoxy-2-oxo-1,2- |
| | dihydroquinolin-8-yloxy)octanamide |
| 61 | 4-(3,5-dichloropyridin-4-ylamino)-8-isobutoxy-7- |
| | methoxyquinolin-2(1H)-one |
| 62 | 8-(3-(1H-imidazol-1-yl)propoxy)-4-(3,5-dichloropyridin-4- |
| | ylamino)-7-methoxyquinolin-2(1H)-one |
| 63 | 1-(6-(4-(3,5-dichloropyridin-4-ylamino)-7-methoxy-2-oxo-1,2- |
| | dihydroquinolin-8-yloxy)hexyl)guanidine |
| 64 | (S)-2-(5-(4-(3,5-dichloropyridin-4-ylamino)-7-methoxy-2-oxo- |
| | 1,2-dihydroquinolin-8-yloxy)pentanamido)-3- |
| | hydroxypropanoic acid |
| 65 | 6-(4-(3,5-dimethylpyridin-4-ylamino)-7-methoxy-2-oxo-1,2- |
| | dihydroquinolin-8-yloxy)hexanamide |
| 66 | (S)-2-(5-(4-(3,5-dichloropyridin-4-ylamino)-7-methoxy-2-oxo- |
| | 1,2-dihydroquinolin-8-yloxy)pentanamido)succinic acid |
| 67 | 8-(3-(2H-tetrazol-5-yl)propoxy)-4-(3,5-dichloropyridin-4- |
| | ylamino)-7-methoxyquinolin-2(1H)-one |
| 68 | 8-(4-(2H-tetrazol-5-yl)butoxy)-4-(3,5-dichloropyridin-4- |
| | ylamino)-7-methoxyquinolin-2(1H)-one |
| 69 | 8-(5-(2H-tetrazol-5-yl)pentyloxy)-4-(3,5-dichloropyridin-4- |
| | ylamino)-7-methoxyquinolin-2(1H)-one |
| 70 | 4-(3,5-dichloropyridin-4-ylamino)-7-methoxy-8-(3-(2-(2- |
| | methoxyethoxy)ethoxy)propoxy)quinolin-2(1H)-one |
| 71 | 8-(6-(2H-tetrazol-5-yl)hexyloxy)-4-(3,5-dichloropyridin-4- |
| | ylamino)-7-methoxyquinolin-2(1H)-one |
| 72 | 5-(4-(3,5-dimethylpyridin-4-ylamino)-7-methoxy-2-oxo-1,2- |
| | dihydroquinolin-8-yloxy)pentanoic acid |
| 73 | 6-(4-(3,5-dimethylpyridin-4-ylamino)-7-methoxy-2-oxo-1,2- |
| | dihydroquinolin-8-yloxy)hexanoic acid |
| 74 | 7-(4-(3,5-dimethylpyridin-4-ylamino)-7-methoxy-2-oxo-1,2- |
| | dihydroquinolin-8-yloxy)heptanoic acid |
| 75 | 8-(4-(3,5-dimethylpyridin-4-ylamino)-7-methoxy-2-oxo-1,2- |
| | dihydroquinolin-8-yloxy)octanoic acid |
| 76 | 9-(4-(3,5-dimethylpyridin-4-ylamino)-7-methoxy-2-oxo-1,2- |
| | dihydroquinolin-8-yloxy)nonanoic acid |
| 77 | 8-(3-(1H-imidazol-1-yl)propoxy)-4-(3,5-dimethylpyridin-4- |
| | ylamino)-7-methoxyquinolin-2(1H)-one |
| 78 | 8-(4-(1H-imidazol-1-yl)butoxy)-4-(3,5-dimethylpyridin-4- |
| | ylamino)-7-methoxyquinolin-2(1H)-one |
| 79 | 8-(5-(1H-imidazol-1-yl)pentyloxy)-4-(3,5-dimethylpyridin-4- |
| | ylamino)-7-methoxyquinolin-2(1H)-one |
| 80 | 8-(6-(1H-imidazol-1-yl)hexyloxy)-4-(3,5-dimethylpyridin-4- |
| | ylamino)-7-methoxyquinolin-2(1H)-one |
| 81 | 4-(3,5-dimethylpyridin-4-ylamino)-8-(3-hydroxypropoxy)-7- |
| | methoxyquinolin-2(1H)-one |
| 82 | 4-(3,5-dimethylpyridin-4-ylamino)-8-(4-hydroxybutoxy)-7- |
| | methoxyquinolin-2(1H)-one |
| 83 | 4-(3,5-dimethylpyridin-4-ylamino)-8-(5-hydroxypentyloxy)-7- |
| | methoxyquinolin-2(1H)-one |
| 84 | 4-(3,5-dimethylpyridin-4-ylamino)-8-(6-hydroxyhexyloxy)-7- |
| | methoxyquinolin-2(1H)-one |
| 85 | 8-(3-(2H-tetrazol-5-yl)propoxy)-4-(3,5-dimethylpyridin-4- |
| | ylamino)-7-methoxyquinolin-2(1H)-one |
| 86 | 8-(4-(2H-tetrazol-5-yl)butoxy)-4-(3,5-dimethylpyridin-4- |
| | ylamino)-7-methoxyquinolin-2(1H)-one |
| 87 | 8-(5-(2H-tetrazol-5-yl)pentyloxy)-4-(3,5-dimethylpyridin-4- |
| | ylamino)-7-methoxyquinolin-2(1H)-one |
| 88 | 8-(6-(2H-tetrazol-5-yl)hexyloxy)-4-(3,5-dimethylpyridin-4- |
| | ylamino)-7-methoxyquinolin-2(1H)-one |
| 89 | 4-(4-(3,5-dimethylpyridin-4-ylamino)-7-methoxy-2-oxo-1,2- |
| | dihydroquinolin-8-yloxy)butanamide |
| 90 | 5-(4-(3,5-dimethylpyridin-4-ylamino)-7-methoxy-2-oxo-1,2- |
| | dihydroquinolin-8-yloxy)pentanamide |
| 91 | 7-(4-(3,5-dimethylpyridin-4-ylamino)-7-methoxy-2-oxo-1,2- |
| | dihydroquinolin-8-yloxy)heptanamide |
| 92 | 8-(4-(3,5-dimethylpyridin-4-ylamino)-7-methoxy-2-oxo-1,2- |
| | dihydroquinolin-8-yloxy)octanamide |
| 93 | 9-(4-(3,5-dimethylpyridin-4-ylamino)-7-methoxy-2-oxo-1,2- |
| | dihydroquinolin-8-yloxy)nonanamide |
| 94 | 2-(5-(4-(3,5-dichloropyridin-4-ylamino)-7-methoxy-2-oxo-1,2- |
| | dihydroquinolin-8-yloxy)pentanamido)acetic acid |
| 95 | (S)-2-(5-(4-(3,5-dichloropyridin-4-ylamino)-7-methoxy-2-oxo- |
| | 1,2-dihydroquinolin-8-yloxy)pentanamido)-3-(1H-imidazol-4- |
| | yl)propanoic acid |
| 96 | 4-(3,5-dichloropyridin-4-ylamino)-8-(3-(2-(2-(2- |
| | hydroxyethoxy)ethoxy)ethoxy)propoxy)-7-methoxyquinolin- |
| | 2(1H)-one |
| 97 | 8-(2,5,8,11-tetraoxatetradecan-14-yloxy)-4-(3,5- |
| | dichloropyridin-4-ylamino)-7-methoxyquinolin-2(1H)-one |
| 98 | 4-(3,5-dichloropyridin-4-ylamino)-8-(3-(2-(2- |
| | hydroxyethoxy)ethoxy)propoxy)-7-methoxyquinolin- |
| | 2(1H)-one |

The following compounds can generally be made using the methods described above. It is expected that these compounds when made will have activity similar to those that have been made in the examples above.

The following compounds are represented herein using the Simplified Molecular Input Line Entry System, or SMILES. SMILES is a modem chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf Comput. Sci. 1988, 28, 31-36. All SMILES strings used herein, as well as numerous IUPAC names, were generated using either CambridgeSoft's ChemDraw 10.0 or ChemBioDraw Ultra 11.0.
COc1ccc2c(cc(═O)[nH]c2c1OCCCCC(═O)O)Nc1c(C)cncc1C
COc1ccc2c(cc(═O)[nH]c2c1OCCCCN1CCN(C)CC1)Nc1c(C)cncc1C
COc1ccc2c(cc(═O)[nH]c2c1OCCCCN1CCOCC1)Nc1c(C)cncc1C
COc1ccc2c(cc(═O)[nH]c2c1OCCCCCC(═O)O)Nc1c(C)cncc1C
COc1ccc2c(cc(═O)[nH]c2c1OCC(═O)N)Nc1c(C)cncc1C
COc1ccc2c(cc(═O)[nH]c2c1OCCCCC(═O)N)Nc1c(C)cncc1C
COc1ccc2c(cc(═O)[nH]c2c1OCCCCCC(═O)O)Nc1c(C)cncc1C
COc1ccc2C(═CC(═O)Nc2c1OCCCCN)Nc3c(C)cncc3C
CNCCCCOc1c(OC)ccc2C(═CC(═O)Nc12)Nc3c(C)cncc3C
COc1ccc2C(═CC(═O)Nc2c1OCCCCCN)Nc3c(C)cncc3C
Cc1cncc(C)c1NC2=CC(═O)Nc3c(OCC(═O)N)c(OCF)ccc23
COc1ccc2C(═CC(═O)Nc2c1OCCCC(═O)N)Nc3c(C)cncc3C
COc1ccc2C(═CC(═O)Nc2c1OCCCCN)Nc3c(C)cncc3F
COc1ccc2C(═CC(═O)Nc2c1OCC(═O)N)Nc3c(C)cncc3F
Cc1cncc(F)c1NC2=CC(═O)Nc3c(OCC(═O)N)c(OCF)ccc23
COc1ccc2C(═CC(═O)Nc2c1OCC(═O)N)Nc3c(C)cncc3Cl
COc1ccc2C(═CC(═O)Nc2c1OCCCCN)Nc3c(F)cncc3F
COc1ccc2C(═CC(═O)Nc2c1OCC(═O)N)Nc3c(F)cncc3F
NC(═O)COc1c(OCF)ccc2C(═CC(═O)Nc12)Nc3c(F)cncc3F
COc1ccc2C(═CC(═O)Nc2c1OCC(═O)N)Nc3c(F)cncc3Cl
COc1ccc2C(═CC(═O)Nc2c1OCCCCN)Nc3c(C)cccc3C
Cc1cccc(C)c1NC2=CC(═O)Nc3c(OCCCCN)c(OCF)ccc23
CNCCCCOc1c(OC)ccc2C(═CC(═O)Nc12)Nc3c(C)cccc3C
COc1ccc2C(═CC(═O)Nc2c1OCCCCCN)Nc3c(C)cccc3C
COc1ccc2C(═CC(═O)Nc2c1OCC(═O)N)Nc3c(C)cccc3C
Cc1cccc(C)c1NC2=CC(═O)Nc3c(OCC(═O)N)c(OCF)ccc23
COc1ccc2C(═CC(═O)Nc2c1OCCCC(═O)N)Nc3c(C)cccc3C
COc1ccc2C(═CC(═O)Nc2c1OCCCCN)Nc3c(C)cccc3F
CNCCCCOc1c(OC)ccc2C(═CC(═O)Nc12)Nc3c(C)cccc3F
COc1ccc2C(═CC(═O)Nc2c1OCCCCCN)Nc3c(C)cccc3F
COc1ccc2C(═CC(═O)Nc2c1OCC(═O)N)Nc3c(C)cccc3F
Cc1cccc(F)c1NC2=CC(═O)Nc3c(OCC(═O)N)c(OCF)ccc23
COc1ccc2C(═CC(═O)Nc2c1OCCCC(═O)N)Nc3c(C)cccc3F
COc1ccc2C(═CC(═O)Nc2c1OCC(═O)N)Nc3c(C)cccc3Cl
COc1ccc2C(═CC(═O)Nc2c1OCC(═O)N)Nc3c(C)cc(F)cc3C
COc1ccc2C(═CC(═O)Nc2c1OCC(═O)N)Nc3c(C)cc(F)cc3F
COc1ccc2C(═CC(═O)Nc2c1OCCCCN)Nc3c(C)cc(O)cc3C
COc1ccc2C(═CC(═O)Nc2c1OCC(═O)N)Nc3c(C)cc(O)cc3C
COc1ccc2C(═CC(═O)Nc2c1OCC(═O)N)Nc3c(C)cc(O)cc3F
COc1ccc2C(═CC(═O)Nc2c1OCCCCN)Nc3c(F)cccc3F
COc1ccc2C(═CC(═O)Nc2c1OCC(═O)N)Nc3c(F)cccc3F
NC(═O)COc1c(OCF)ccc2C(═CC(═O)Nc12)Nc3c(F)cccc3F
COc1ccc2C(═CC(═O)Nc2c1OCC(═O)N)Nc3c(F)cccc3Cl
COc1ccc2C(═CC(═O)Nc2c1OCC(═O)N)Nc3c(F)cc(F)cc3F
COc1ccc2C(═CC(═O)Nc2c1OCC(═O)N)Nc3c(F)cc(O)cc3F
CN(C)CCCCOc1c(OC(F)F)ccc2C(═CC(═O)Nc12)Nc3c(C)cccc3C
CCCN(C)CCCCOc1c(OCF)ccc2C(═CC(═O)Nc12)Nc3c(C)cccc3C
Cc1cccc(C)c1NC2=CC(═O)Nc3c(OCCCCn4cccn4)c(OC(F)(F)F)ccc23
COCCOCCOCCOCCCCOc1c(OC)ccc2C(═CC(═O)Nc12)Nc3c(C)cccc3C
Cc1cccc(C)c1NC2=CC(═O)Nc3c(OCCCCCN)c(OC(F)F)ccc23
COc1ccc2C(═CC(═O)Nc2c1OCCCCCn3ccnc3)Nc4c(C)cccc4C
Cc1cccc(C)c1NC2=CC(═O)Nc3c(OCCCCCCNC(═N)N)c(OC(F)(F)F)ccc23
Cc1cccc(C)c1NC2=CC(═O)Nc3c(OCCCCCCN4CCCCC4)c(OC(F)F)ccc23
Cc1cccc(C)c1NC2=CC(═O)Nc3c(OCCCCCCn4ccnn4)c(OCF)ccc23
CC(C)[C@H](NC(═O)COc1c(OCF)ccc2C(═CC(═O)Nc12)Nc3c(C)cccc3C)C(═O)O
Cc1cccc(C)c1NC2=CC(═O)Nc3c(OCC(═O)N[C@@H](Cc4ccccc4)C(═O)O)c(OC(F)F)ccc23
COc1ccc2C(═CC(═O)Nc2c1OCC(═O)N[C@@H](CCSC)C(═O)O)Nc3c(C)cccc3C
Cc1cccc(C)c1NC2=CC(═O)Nc3c(OCC(═O)N[C@@H](CCCNC(═N)N)C(═O)O)c(OC (F)(F)F)ccc23
Cc1cccc(C)c1NC2=CC(═O)Nc3c(OCCCC(═O)NCC(═O)O)c(OC(F)(F)F)ccc23
COc1ccc2C(═CC(═O)Nc2c1OCCCC(═O)N[C@@H](CCCCN)C(═O)O)Nc3c(C)cccc3C
Cc1cccc(C)c1NC2=CC(═O)Nc3c(OCCCCC(═O)N[C@@H](CCCCN)C(═O)O)c(OCF)ccc23
Cc1cccc(C)c1NC2=CC(═O)Nc3c(OCCCCC(═O)N[C@@H](Cc4cnc[nH]4)C(═O)O)c(OC(F)F)ccc23
CN1CCN(CC1)C(═O)CCCCCOc2c(OC(F)(F)F)ccc3C(═CC(═O)Nc23)Nc4c(C)cccc4C
Cc1cccc(C)c1NC2=CC(═O)Nc3c(OCCCCCC(═O)N[C@@H](Cc4ccc(O)cc4)C(═O)O)c(OCF)ccc23
Cc1cccc(C)c1NC2=CC(═O)Nc3c(OCCCCCC(═O)N[C@@H](Cc4ccc(O)cc4)C(═O)O)c(OC(F)F)ccc23
CCNCCCCOc1c(OC)ccc2C(═CC(═O)Nc12)Nc3c(C)cccc3F
CCN(C)CCCCOc1c(OCF)ccc2C(═CC(═O)Nc12)Nc3c(C)cccc3F
COCCN(C)CCCCOc1c(OC(F)(F)F)ccc2C(═CC(═O)Nc12)Nc3c(C)cccc3F
COc1ccc2C(═CC(═O)Nc2c1OCCCCCNC(═N)N)Nc3c(C)cccc3F
Cc1cccc(F)c1NC2=CC(═O)Nc3c(OCCCCCn4cnnc4)c(OC(F)F)ccc23
Cc1cccc(F)c1NC2=CC(═O)Nc3c(OCCCCCOc4ccncc4)c(OCF)ccc23
CCNCCCCCCOc1c(OC(F)(F)F)ccc2C(═CC(═O)Nc12)Nc3c(C)cccc3F
Cc1cccc(F)c1NC2=CC(═O)Nc3c(OCCCCCCN4CCCC(F)C4)c(OCF)ccc23
COc1ccc2C(═CC(═O)Nc2c1OCCCCCCOc3cccnc3)Nc4c(C)cccc4F
Cc1cccc(F)c1NC2=CC(═O)Nc3c(OCC(═O)N)c(OC(F)(F)F)ccc23
CN(C)CCOC(═O)COc1c(OCF)ccc2C(═CC(═O)Nc12)Nc3c(C)cccc3F
Cc1cccc(F)c1NC2=CC(═O)Nc3c(OCC(═O)N[C@@H](CO)C(═O)O)c(OC(F)F)ccc23
Cc1cccc(F)c1NC2=CC(═O)Nc3c(OCCCC(═O)N[C@@H](Cc4c[nH]c5ccccc45)C(═O) O)c(OCF)ccc23
Cc1cccc(F)c1NC2=CC(═O)Nc3c(OCCCC(═O)N[C@@H](Cc4c[nH]c5ccccc45)C(═O) O)c(OC(F)F)ccc23
Cc1cccc(F)c1NC2=CC(═O)Nc3c(OCCCCC(═O)OCC[N+](C)(C)C)c(OC(F)(F)F)ccc2 3
CC(C)[C@H](NC(═O)CCCCOc1c(OC(F)F)ccc2C(═CC(═O)Nc12)Nc3c(C)cccc3F)C(═O)O
COc1ccc2C(═CC(═O)Nc2c1OCCCCC(═O)N[C@@H](CCCCN)C(═O)O)Nc3c(C)ccc c3F
Cc1cccc(F)c1NC2=CC(═O)Nc3c(OCCCCCC(═O)N)c(OCF)ccc23
CN1CCN(CC1)C(═O)CCCCCOc2c(OC(F)F)ccc3C(═CC(═O)Nc23)Nc4c(C)cccc4F
CC[C@H](C)[C@H](NC(═O)CCCCCOc1c(OC)ccc2C(═CC(═O)Nc12)Nc3c(C)cccc3F)C(═O)O
COc1ccc2C(═CC(═O)Nc2c1OCCCCN3CCOCC3)Nc4c(C)cccc4Cl
Cc1cccc(Cl)c1NC2=CC(═O)Nc3c(OCCCCN4CCC(F)CC4)c(OC(F)F)ccc23
Cc1cccc(Cl)c1NC2=CC(═O)Nc3c(OCCCCOc4ccccn4)c(OC(F)(F)F)ccc23
Cc1cccc(Cl)c1NC2=CC(═O)Nc3c(OCCCCCNC(═N)N)c(OCF)ccc23
CCCCCN(C)CCCCCOc1c(OC)ccc2C(═CC(═O)Nc12)Nc3c(C)cccc3Cl
Cc1cccc(Cl)c1NC2=CC(═O)Nc3c(OCCCCCOc4cccnc4)c(OC(F)F)ccc23
COc1ccc2C(═CC(═O)Nc2c1OCCCCCCN)Nc3c(C)cccc3Cl
Cc1cccc(Cl)c1NC2=CC(═O)Nc3c(OCCCCCCN4CCOCC4)c(OC(F)(F)F)ccc23
COCCOCCOCCCCCCOc1c2NC(═O)C═C(Nc3c(C)cccc3Cl)c2ccc10C(F)F
COc1ccc2C(═CC(═O)Nc2c1OCC(═O)N[C@@H](C(C)C)C(═O))Nc3c(C)cccc3Cl
CC(C)C[C@H](NC(═O)CCCOc1c(OC(F)F)ccc2C(═CC(═O)Nc12)Nc3c(C)cccc3Cl)C (═O)O
COc1ccc2C(═CC(═O)Nc2c1OCCCC(═O)N[C@@H](Cc3c[nH]c4ccccc34)C(═O)O)N c5c(C)cccc5Cl
Cc1cccc(Cl)c1NC2=CC(═O)Nc3c(OCCCC(═O)N[C@@H](CCCNC(═N)N)C(═O)O)c (OCF)ccc23
CC(C)[C@H](NC(═O)CCCCOc1c(OC(F)F)ccc2C(═CC(═O)Nc12)Nc3c(C)cccc3Cl)C (═O)O
COc1ccc2C(═CC(═O)Nc2c1OCCCCC(═O)N[C@@H](CCCNC(═N)N)C(═O)O)Nc3c (C)cccc3Cl
CC(C)C[C@H](NC(═O)CCCCCOc1c(OCF)ccc2C(═CC(═O)Nc12)Nc3c(C)cccc3Cl)C (═O)O
Cc1cccc(Br)c1NC2=CC(═O)Nc3c(OCCCCN)c(OC(F)F)ccc23
COCCN(C)CCCCOc1c(OC(F)(F)F)ccc2C(═CC(═O)Nc12)Nc3c(C)cccc3Br
COc1ccc2C(═CC(═O)Nc2c1OCCCCN3CCC(F)C3)Nc4c(C)cccc4Br
Cc1cccc(Br)c1NC2=CC(═O)Nc3c(OCCCCCN4CCCC(F)C4)c(OC(F)F)ccc23
Cc1cccc(Br)c1NC2=CC(═O)Nc3c(OCCCCCn4cccc4)c(OC(F)(F)F)ccc23
CCCNCCCCCCOc1c(OC(F)F)ccc2C(═CC(═O)Nc12)Nc3c(C)cccc3Br
COc1ccc2C(═CC(═O)Nc2c1OCCCCCCn3cnnn3)Nc4c(C)cccc4Br
COCCOCCOCCCCCCOc1c(OC(F)(F)F)ccc2C(═CC(═O)Nc12)Nc3c(C)cccc3Br
COc1ccc2C(═CC(═O)Nc2c1OCC(═O)N[C@@H](CO)C(═O)O)Nc3c(C)cccc3Br
Cc1cccc(Br)c1NC2=CC(═O)Nc3c(OCC(═O)N[C@@H](CCCCN)C(═O)O)c(OC(F)F) ccc23
Cc1cccc(Br)c1NC2=CC(═O)Nc3c(OCC(═O)N[C@@H](CCC(═O)O)C(═O)O)c(OCF) ccc23
CN(C)CCN(C)C(═O)CCCOc1c(OC(F)(F)F)ccc2C(═CC(═O)Nc12)Nc3c(C)cccc3Br
CN1CCN(CC1)C(═O)CCCOc2c(OC(F)F)ccc3C(═CC(═O)Nc23)Nc4c(C)cccc4Br
COc1ccc2C(═CC(═O)Nc2c1OCCCC(═O)N[C@@H](CO)C(═O)O)Nc3c(C)cccc3Br
CN(C)CCCN(C)C(═O)CCCCOc1c(OC(F)F)ccc2C(═CC(═O)Nc12)Nc3c(C)cccc3Br
CN(CCC[N+](C)(C)C)C(═O)CCCCOc1c(OC(F)(F)F)ccc2C(═CC(═O)Nc12)Nc3c(C)c ccc3Br
CN(CCC[N+](C)(C)C)C(═O)CCCCCOc1c(OC(F)F)ccc2C(═CC(═O)Nc12)Nc3c(C)cc cc3Br
COc1ccc2C(═CC(═O)Nc2c1OCCCCCC(═O)N[C@@H](Cc3ccc(O)cc3)C(═O)O)Nc4c (C)cccc4Br
CCCN(C)CCCCOc1c(OC(F)(F)F)ccc2C(═CC(═O)Nc12)Nc3c(C)cc(F)cc3C
COc1ccc2C(═CC(═O)Nc2c1OCCCCN3CCCCC3)Nc4c(C)cc(F)cc4C
Cc1cc(F)cc(C)c1NC2=CC(═O)Nc3c(OCCCCN4CCCC4)c(OCF)ccc23
CCCCCN(C)CCCCCOc1c(OC)ccc2C(═CC(═O)Nc12)Nc3c(C)cc(F)cc3C
Cc1cc(F)cc(C)c1NC2=CC(═O)Nc3c(OCCCCCn4nccn4)c(OC(F)F)ccc23
Cc1cc(F)cc(C)c1NC2=CC(═O)Nc3c(OCCCCCOc4ccncc4)c(OC(F)(F)F)ccc23
Cc1cc(F)cc(C)c1NC2=CC(═O)Nc3c(OCCCCCCn4ccnc4)c(OC(F)F)ccc23
Cc1cc(F)cc(C)c1NC2=CC(═O)Nc3c(OCC(═O)OCC[N+](C)(C)C)c(OCF)ccc23
CN(C)CCCN(C)C(═O)CCCOc1c(OCF)ccc2C(═CC(═O)Nc12)Nc3c(C)cc(F)cc3C
Cc1cc(F)cc(C)c1NC2=CC(═O)Nc3c(OCCCCC(═O)OCC[N+](C)(C)C)c(OC(F)(F)F)c cc23
Cc1cc(F)cc(C)c1NC2=CC(═O)Nc3c(OCCCCC(═O)N4CCC[C@H]4C(═O)O)c(OCF)c cc23
COc1ccc2C(═CC(═O)Nc2c1OCCCCCC(═O)N(C)CCN(C)C)Nc3c(C)cc(F)cc3C
Cc1cc(F)cc(C)c1NC2=CC(═O)Nc3c(OCCCCCC(═O)N4CCC[C@H]4C(═O)O)c(OC(F)(F)F)ccc23
Cc1cc(F)cc(C)c1NC2=CC(═O)Nc3c(OCCCCCC(═O)N[C@@H](Cc4ccccc4)C(═O)O) c(OCF)ccc23
Cc1cc(F)cc(F)c1NC2=CC(═O)Nc3c(OCCCCn4cnnn4)c(OC(F)F)ccc23
Cc1cc(F)cc(F)c1NC2=CC(═O)Nc3c(OCCCCOc4ccncc4)c(OC(F)(F)F)ccc23
COc1ccc2C(═CC(═O)Nc2c1OCCCCO[C@@H]3O[C@H](CO)[C@@H](O)[C@H](O)[C@H]3O)Nc4c(C)cc(F)cc4F
CCCCN(C)CCCCCOc1c(OC(F)F)ccc2C(═CC(═O)Nc12)Nc3c(C)cc(F)cc3F
Cc1cc(F)cc(F)c1NC2=CC(═O)Nc3c(OCCCCCOc4ccncc4)c(OCF)ccc23
COc1ccc2C(═CC(═O)Nc2c1OCCCCCCN)Nc3c(C)cc(F)cc3F
COCCN(C)CCCCCCOc1c(OC(F)(F)F)ccc2C(═CC(═O)Nc12)Nc3c(C)cc(F)cc3F
Cc1cc(F)cc(F)c1NC2=CC(═O)Nc3c(OCCCCCCN4CCCCC4)c(OCF)ccc23
Cc1cc(F)cc(F)c1NC2=CC(═O)Nc3c(OCCCCCCn4nccn4)c(OC(F)F)ccc23
CC(C)C[C@H](NC(═O)COc1c(OC(F)(F)F)ccc2C(═CC(═O)Nc12)Nc3c(C)cc(F)cc3F) C(═O)O
CC(C)[C@H](NC(═O)CCCOc1c(OC(F)(F)F)ccc2C(═CC(═O)Nc12)Nc3c(C)cc(F)cc3F)C(═O)O
COc1ccc2C(═CC(═O)Nc2c1OCCCC(═O)N[C@@H](CCCCN)C(═O)O)Nc3c(C)cc(F) cc3F
CN(C)CCN(C)C(═O)CCCCOc1c(OC(F)F)ccc2C(═CC(═O)Nc12)Nc3c(C)cc(F)cc3F
COc1ccc2C(═CC(═O)Nc2c1OCCCCC(═O)N3CCNCC3)Nc4c(C)cc(F)cc4F
Cc1cc(F)cc(F)c1NC2=CC(═O)Nc3c(OCCCCC(═O)N[C@@H](Cc4ccc(O)cc4)C(═O) O)c(OCF)ccc23
Cc1cc(F)cc(F)c1NC2=CC(═O)Nc3c(OCCCCC(═O)N[C@@H](CCC(═O)N)C(═O)O)c (OC(F)(F)F)ccc23
CC(C)[C@H](NC(═O)CCCCCOc1c(OC(F)F)ccc2C(═CC(═O)Nc12)Nc3c(C)cc(F)cc3F)C(═O)O
Cc1cc(F)cc(F)c1NC2=CC(═O)Nc3c(OCCCCCC(═O)N[C@@H](Cc4ccccc4)C(═O)O) c(OCF)ccc23
CCCCN(C)CCCCOc1c(OCF)ccc2C(═CC(═O)Nc12)Nc3c(C)cc(F)cc3Cl
COc1ccc2C(═CC(═O)Nc2c1OCCCCn3ccnc3)Nc4c(C)cc(F)cc4Cl
Cc1cc(F)cc(Cl)c1NC2=CC(═O)Nc3c(OCCCCOc4cccnc4)c(OC(F)(F)F)ccc23
COCCN(C)CCCCCOc1c(OCF)ccc2C(═CC(═O)Nc12)Nc3c(C)cc(F)cc3Cl
Cc1cc(F)cc(Cl)c1NC2=CC(═O)Nc3c(OCCCCCn4ncnn4)c(OC(F)(F)F)ccc23
COc1ccc2C(═CC(═O)Nc2c1OCCCCCOc3cccnc3)Nc4c(C)cc(F)cc4Cl
Cc1cc(F)cc(Cl)c1NC2=CC(═O)Nc3c(OCCCCCOc4ccncc4)c(OC(F)F)ccc23
CCCCN(C)CCCCCCOc1c(OC)ccc2C(═CC(═O)Nc12)Nc3c(C)cc(F)cc3Cl
Cc1cc(F)cc(Cl)c1NC2=CC(═O)Nc3c(OCCCCCCN4CCC(F)C4)c(OCF)ccc23
Cc1cc(F)cc(Cl)c1NC2=CC(═O)Nc3c(OCCCCCCn4cccc4)c(OC(F)F)ccc23
Cc1cc(F)cc(Cl)c1NC2=CC(═O)Nc3c(OCC(═O)NCC(═O)O)c(OC(F)(F)F)ccc23
Cc1cc(F)cc(Cl)c1NC2=CC(═O)Nc3c(OCC(═O)N[C@@H](CO)C(═O)O)c(OCF)ccc23
CN(CCC[N+](C)(C)C)C(═O)CCCOc1c(OCF)ccc2C(═CC(═O)Nc12)Nc3c(C)cc(F)cc3Cl
COc1ccc2C(═CC(═O)Nc2c1OCCCC(═O)N[C@@H](CCCCN)C(═O)O)Nc3c(C)cc(F) cc3Cl
Cc1cc(F)cc(Cl)c1NC2=CC(═O)Nc3c(OCCCC(═O)N[C@@H](CCCCN)C(═O)O)c(O C(F)F)ccc23
Cc1cc(F)cc(Cl)c1NC2=CC(═O)Nc3c(OCCCCC(═O)N[C@@H](CO)C(═O)O)c(OCF) ccc23
COc1ccc2C(═CC(═O)Nc2c1OCCCCC(═O)N[C@@H]([C@@H](C)O)C(═O)O)Nc3c(C)cc(F)cc3Cl
Cc1cc(F)cc(Cl)c1NC2=CC(═O)Nc3c(OCCCCC(═O)N[C@@H](CCCCN)C(═O)O)c(OC(F)(F)F)ccc23
Cc1cc(F)cc(Cl)c1NC2=CC(═O)Nc3c(OCCCCC(═O)N[C@@H](CCCNC(═N)N)C(═O)O)c(OC(F)F)ccc23
Cc1cc(F)cc(Cl)c1NC2=CC(═O)Nc3c(OCCCCCC(═O)OCC[N+](C)(C)C)c(OCF)ccc23
Cc1cc(F)cc(Cl)c1NC2=CC(═O)Nc3c(OCCCCCC(═O)N[C@@H](CO)C(═O)O)c(OC(F)(F)F)ccc23
COCCN(C)CCCCOc1c(OC(F)(F)F)ccc2C(═CC(═O)Nc12)Nc3c(C)cc(F)cc3Br
Cc1cc(F)cc(Br)c1NC2=CC(═O)Nc3c(OCCCCn4ccnn4)c(OC(F)F)ccc23
CCCCCN(C)CCCCCOc1c(OCF)ccc2C(═CC(═O)Nc12)Nc3c(C)cc(F)cc3Br
COc1ccc2C(═CC(═O)Nc2c1OCCCCCN3CCC(F)CC3)Nc4c(C)cc(F)cc4Br
COCCOCCCCCOc1c(OC(F)F)ccc2C(═CC(═O)Nc12)Nc3c(C)cc(F)cc3Br
Cc1cc(F)cc(Br)c1NC2=CC(═O)Nc3c(OCCCCCCN4CCCC(F)(F)C4)c(OC(F)(F)F)ccc 23
Cc1cc(F)cc(Br)c1NC2=CC(═O)Nc3c(OCCCCCCn4ccnc4)c(OC(F)F)ccc23
Cc1cc(F)cc(Br)c1NC2=CC(═O)Nc3c(OCC(═O)N[C@@H](CCC(═O)O)C(═O)O)c(O C(F)(F)F)ccc23
COc1ccc2C(═CC(═O)Nc2c1OCCCC(═O)N[C@@H](CCC(═O)N)C(═O)O)Nc3c(C)cc (F)cc3Br
Cc1cc(F)cc(Br)c1NC2=CC(═O)Nc3c(OCCCCC(═O)OCC[N+](C)(C)C)c(OCF)ccc23
COc1ccc2C(═CC(═O)Nc2c1OCCCCC(═O)N[C@@H](CCCCN)C(═O)O)Nc3c(C)cc(F)cc3Br
Cc1cc(F)cc(Br)c1NC2=CC(═O)Nc3c(OCCCCC(═O)N[C@@H](CCCCN)C(═O)O)c(OC(F)F)ccc23
Cc1cc(F)cc(Br)c1NC2=CC(═O)Nc3c(OCCCCC(═O)N[C@@H](CCCCN)C(═O)O)c(OC(F)(F)F)ccc23
Cc1cc(F)cc(Br)c1NC2=CC(═O)Nc3c(OCCCCCC(═O)OCC[N+](C)(C)C)c(OCF)ccc2 3
Cc1cc(F)cc(Br)c1NC2=CC(═O)Nc3c(OCCCCCC(═O)N[C@@H](CO)C(═O)O)c(OC(F)F)ccc23
COc1ccc2C(═CC(═O)Nc2c1OCCCCCC(═O)N[C@@H](CCCNC(═N)N)C(═O)O)Nc3c(C)cc(F)cc3Br
CCCNCCCCOc1c(OC(F)F)ccc2C(═CC(═O)Nc12)Nc3c(C)cc(O)cc3C
Cc1cc(O)cc(C)c1NC2=CC(═O)Nc3c(OCCCCN4CCC(F)(F)C4)c(OCF)ccc23
COc1ccc2C(═CC(═O)Nc2c1OCCCCn3cnnc3)Nc4c(C)cc(O)cc4C
CCCNCCCCCOc1c(OC)ccc2C(═CC(═O)Nc12)Nc3c(C)cc(O)cc3C
Cc1cc(O)cc(C)c1NC2=CC(═O)Nc3c(OCCCCCn4cncn4)c(OC(F)(F)F)ccc23
Cc1cc(O)cc(C)c1NC2=CC(═O)Nc3c(OCCCCCn4cnnn4)c(OC(F)F)ccc23
Cc1cc(O)cc(C)c1NC2=CC(═O)Nc3c(OCCCCCOc4ccncc4)c(OCF)ccc23
COCCN(C)CCCCCCOc1c(OCF)ccc2C(═CC(═O)Nc12)Nc3c(C)cc(O)cc3C
Cc1cc(O)cc(C)c1NC2=CC(═O)Nc3c(OCCCCCCN4CCOCC4)c(OC(F)F)ccc23
COc1ccc2C(═CC(═O)Nc2c1OCCCCCCN3CCCCC3)Nc4c(C)cc(O)cc4C
Cc1cc(O)cc(C)c1NC2=CC(═O)Nc3c(OCC(═O)N[C@@H](CC(═O)O)C(═O)O)c(OCF)ccc23
COc1ccc2C(═CC(═O)Nc2c1OCC(═O)N[C@@H](CC(═O)N)C(═O)O)Nc3c(C)cc(O)c c3C
CN(C)CCCN(C)C(═O)CCCOc1c(OCF)ccc2C(═CC(═O)Nc12)Nc3c(C)cc(O)cc3C
CN(CCC[N+](C)(C)C)C(═O)CCCCOc1c(OC(F)(F)F)ccc2C(═CC(═O)Nc12)Nc3c(C)c c(O)cc3C
C[C@H](NC(═O)CCCCOc1c(OC(F)F)ccc2C(═CC(═O)Nc12)Nc3c(C)cc(O)cc3C)C(═O)O
COc1ccc2C(═CC(═O)Nc2c1OCCCCCC(═O)N[C@@H](Cc3ccccc3)C(═O)O)Nc4c(C) cc(O)cc4C
Cc1cc(O)cc(C)c1NC2=CC(═O)Nc3c(OCCCCCC(═O)N[C@@H](CO)C(═O)O)c(OC(F)(F)F)ccc23
Cc1cc(O)cc(F)c1NC2=CC(═O)Nc3c(OCCCCn4cccn4)c(OCF)ccc23
COCCOCCCCOc1c(OC(F)(F)F)ccc2C(═CC(═O)Nc12)Nc3c(C)cc(O)cc3F
CCCN(C)CCCCCOc1c(OCF)ccc2C(═CC(═O)Nc12)Nc3c(C)cc(O)cc3F
Cc1cc(O)cc(F)c1NC2=CC(═O)Nc3c(OCCCCCn4cccn4)c(OC(F)F)ccc23
Cc1cc(O)cc(F)c1NC2=CC(═O)Nc3c(OCCCCCn4ncnn4)c(OC(F)(F)F)ccc23
CNCCCCCCOc1c(OCF)ccc2C(═CC(═O)Nc12)Nc3c(C)cc(O)cc3F
COCCOCCCCCCOc1c(OC)ccc2C(═CC(═O)Nc12)Nc3c(C)cc(O)cc3F
Cc1cc(O)cc(F)c1NC2=CC(═O)Nc3c(OCC(═O)N)c(OC(F)(F)F)ccc23
COc1ccc2C(═CC(═O)Nc2c1OCC(═O)N[C@@H](CC(C)C)C(═O)O)Nc3c(C)cc(O)cc3F
Cc1cc(O)cc(F)c1NC2=CC(═O)Nc3c(OCC(═O)N[C@@H](CO)C(═O)O)c(OCF)ccc23
Cc1cc(O)cc(F)c1NC2=CC(═O)Nc3c(OCCCC(═O)N4CCNCC4)c(OC(F)F)ccc23
CN1CCN(CC1)C(═O)CCCOc2c(OCF)ccc3C(═CC(═O)Nc23)Nc4c(C)cc(O)cc4F
Cc1cc(O)cc(F)c1NC2=CC(═O)Nc3c(OCCCC(═O)N[C@@H](CCCNC(═N)N)C(═O) O)c(OC(F)(F)F)ccc23
COc1ccc2C(═CC(═O)Nc2c1OCCCCC(═O)N3CCNCC3)Nc4c(C)cc(O)cc4F
CC(C)[C@H](NC(═O)CCCCOc1c(OCF)ccc2C(═CC(═O)Nc12)Nc3c(C)cc(O)cc3F)C(═O)O
COc1ccc2C(═CC(═O)Nc2c1OCCCCCC(═O)NCC[N+](C)(C)C)Nc3c(C)cc(O)cc3F
Cc1cc(O)cc(F)c1NC2=CC(═O)Nc3c(OCCCCCC(═O)N[C@@H](Cc4ccc(O)cc4)C(═O)O)c(OC(F)F)ccc23
CCN(CC)CCCCOc1c(OC)ccc2C(═CC(═O)Nc12)Nc3c(C)cc(O)cc3Cl
Cc1cc(O)cc(Cl)c1NC2=CC(═O)Nc3c(OCCCCN4CCCCC4)c(OC(F)(F)F)ccc23
Cc1cc(O)cc(Cl)c1NC2=CC(═O)Nc3c(OCCCCN4CCC(F)C4)c(OC(F)F)ccc23
CCNCCCCCOc1c(OCF)ccc2C(═CC(═O)Nc12)Nc3c(C)cc(O)cc3Cl
CCCN(C)CCCCCOc1c(OC(F)F)ccc2C(═CC(═O)Nc12)Nc3c(C)cc(O)cc3Cl
COc1ccc2C(═CC(═O)Nc2c1OCCCCCn3cnnc3)Nc4c(C)cc(O)cc4Cl
Cc1cc(O)cc(Cl)c1NC2=CC(═O)Nc3c(OCCCCCOCCOCCOCC)c(OC(F)(F)F)ccc23
CCN(CC)CCCCCCOc1c(OC)ccc2C(═CC(═O)Nc12)Nc3c(C)cc(O)cc3Cl
Cc1cc(O)cc(Cl)c1NC2=CC(═O)Nc3c(OCCCCCCn4ccnc4)c(OCF)ccc23
Cc1cc(O)cc(Cl)c1NC2=CC(═O)Nc3c(OCC(═O)OCC[N+](C)(C)C)c(OC(F)F)ccc23
Cc1cc(O)cc(Cl)c1NC2=CC(═O)Nc3c(OCC(═O)N4CCC[C@H]4C(═O)O)c(OCF)ccc2 3
C[C@H](NC(═O)CCCCOc1c(OCF)ccc2C(═CC(═O)Nc12)Nc3c(C)cc(O)cc3Cl)C(═O) O
C[C@@H](O)[C@H](NC(═O)CCCCOc1c(OC(F)F)ccc2C(═CC(═O)Nc12)Nc3c(C)cc(O)cc3Cl)C(═O)O
CN(CCC[N+](C)(C)C)C(═O)CCCCCOc1c(OCF)ccc2C(═CC(═O)Nc12)Nc3c(C)cc(O) cc3Cl
Cc1cc(O)cc(Cl)c1NC2=CC(═O)Nc3c(OCCCCCC(═O)N4CCNCC4)c(OC(F)F)ccc23
Cc1cc(O)cc(Cl)c1NC2=CC(═O)Nc3c(OCCCCCC(═O)N[C@@H](CO)C(═O)O)c(OC(F)(F)F)ccc23
COc1ccc2C(═CC(═O)Nc2c1OCCCCCC(═O)N[C@@H](CC(═O)N)C(═O)O)Nc3c(C) cc(O)cc3Cl
Cc1cc(O)cc(Br)c1NC2=CC(═O)Nc3c(OCCCCn4nccn4)c(OC(F)(F)F)ccc23
COc1ccc2C(═CC(═O)Nc2c1OCCCCOCCOCCOCC)Nc3c(C)cc(O)cc3Br
Cc1cc(O)cc(Br)c1NC2=CC(═O)Nc3c(OCCCCCn4cnnc4)c(OC(F)F)ccc23
Cc1cc(O)cc(Br)c1NC2=CC(═O)Nc3c(OCCCCCOc4ccccn4)c(OC(F)(F)F)ccc23
COc1ccc2C(═CC(═O)Nc2c1OCCCCCOc3ccncc3)Nc4c(C)cc(O)cc4Br
CCN(C)CCCCCCOc1c(OC(F)F)ccc2C(═CC(═O)Nc12)Nc3c(C)cc(O)cc3Br
Cc1cc(O)cc(Br)c1NC2=CC(═O)Nc3c(OCCCCCCn4ncnn4)c(OCF)ccc23
Cc1cc(O)cc(Br)c1NC2=CC(═O)Nc3c(OCCCCCCCCCC)c(OC(F)(F)F)ccc2 3
CC[C@H](C)[C@H](NC(═O)COc1c(OC(F)F)ccc2C(═CC(═O)Nc12)Nc3c(C)cc(O)cc 3Br)C(═O)O
Cc1cc(O)cc(Br)c1NC2=CC(═O)Nc3c(OCC(═O)N[C@@H](Cc4ccc(O)cc4)C(═O)O)c(OCF)ccc23
Cc1cc(O)cc(Br)c1NC2=CC(═O)Nc3c(OCC(═O)N[C@@H](CCCNC(═N)N)C(═O)O)c (OC(F)(F)F)ccc23
Cc1cc(O)cc(Br)c1NC2=CC(═O)Nc3c(OCCCC(═O)N4CCOC[C@H]4C(═O)O)c(OC(F) F)ccc23
CN(C)CCN(C)C(═O)CCCCOc1c(OC(F)(F)F)ccc2C(═CC(═O)Nc12)Nc3c(C)cc(O)cc3Br
COc1ccc2C(═CC(═O)Nc2c1OCCCCC(═O)N[C@@H](CC(═O)O)C(═O)O)Nc3c(C)cc (O)cc3Br
Cc1cc(O)cc(Br)c1NC2=CC(═O)Nc3c(OCCCCCC(═O)N[C@@H](CCCNC(═N)N)C(═O)O)c(OCF)ccc23
Cc1cc(cc(C)c1NC2=CC(═O)Nc3c(OCCCCn4cncn4)c(OC(F)F)ccc23)C(═O)O
Cc1cc(cc(C)c1NC2=CC(═O)Nc3c(OCCCCn4cnnc4)c(OC(F)(F)F)ccc23)C(═O)O
Cc1cc(cc(C)c1NC2=CC(═O)Nc3c(OCCCCOCCO)c(OCF)ccc23)C(═O)O
COCCOCCOCCOCCCCOc1c(OC)ccc2C(═CC(═O)Nc12)Nc3c(C)cc(cc3C)C(═O)O
Cc1cc(cc(C)c1NC2=CC(═O)Nc3c(OCCCCCn4cccn4)c(OC(F)F)ccc23)C(═O)O
Cc1cc(cc(C)c1NC2=CC(═O)Nc3c(OCCCCCn4ncnn4)c(OCF)ccc23)C(═O)O
COc1ccc2C(═CC(═O)Nc2c1OCCCCCOc3ccncc3)Nc4c(C)cc(cc4C)C(═O)O
Cc1cc(cc(C)c1NC2=CC(═O)Nc3c(OCCCCCCN4CCOCC4)c(OC(F)F)ccc23)C(═O)O
Cc1cc(cc(C)c1NC2=CC(═O)Nc3c(OCCCCCCOCCO)c(OC(F)(F)F)ccc23)C(═O)O
CN(C)CCNC(═O)COc1c(OCF)ccc2C(═CC(═O)Nc12)Nc3c(C)cc(cc3C)C(═O)O
COc1ccc2C(═CC(═O)Nc2c1OCC(═O)N(C)CCCN(C)C)Nc3c(C)cc(cc3C)C(═O)O
Cc1cc(cc(C)c1NC2=CC(═O)Nc3c(OCC(═O)N4CCNCC4)c(OC(F)F)ccc23)C(═O)O
Cc1cc(cc(C)c1NC2=CC(═O)Nc3c(OCC(═O)N[C@@H](CO)C(═O)O)c(OC(F)(F)F)cc c23)C(═O)O
Cc1cc(cc(C)c1NC2=CC(═O)Nc3c(OCCCC(═O)N[C@@H](CCCNC(═N)N)C(═O)O)c (OC(F)F)ccc23)C(═O)O
C[C@@H](O)[C@H](NC(═O)CCCCOc1c(OCF)ccc2C(═CC(═O)Nc12)Nc3c(C)cc(cc 3C)C(═O)O)C(═O)O
CN(C)CCOC(═O)CCCCCOc1c(OC(F)(F)F)ccc2C(═CC(═O)Nc12)Nc3c(C)cc(cc3C)C (═O)O
CN(C)CCNC(═O)CCCCCOc1c(OCF)ccc2C(═CC(═O)Nc12)Nc3c(C)cc(cc3C)C(═O)O
Cc1cc(cc(F)c1NC2=CC(═O)Nc3c(OCCCCN4CCCC(F)(F)C4)c(OCF)ccc23)C(═O)O
Cc1cc(cc(F)c1NC2=CC(═O)Nc3c(OCCCCOc4ccccn4)c(OC(F)F)ccc23)C(═O)O
COc1ccc2C(═CC(═O)Nc2c1OCCCCCOc3cccnc3)Nc4c(C)cc(cc4F)C(═O)O
Cc1cc(cc(F)c1NC2=CC(═O)Nc3c(OCCCCCOc4ccncc4)c(OC(F)F)ccc23)C(═O)O
CCCNCCCCCCOc1c(OC(F)F)ccc2C(═CC(═O)Nc12)Nc3c(C)cc(cc3F)C(═O)O
Cc1cc(cc(F)c1NC2=CC(═O)Nc3c(OCCCCCCn4cnnc4)c(OC(F)(F)F)ccc23)C(═O)O
Cc1cc(cc(F)c1NC2=CC(═O)Nc3c(OCCCCCCOc4ccccn4)c(OCF)ccc23)C(═O)O
COc1ccc2C(═CC(═O)Nc2c1OCCCCCCOc3ccncc3)Nc4c(C)cc(cc4F)C(═O)O
Cc1cc(cc(F)c1NC2=CC(═O)Nc3c(OCC(═O)N)c(OC(F)F)ccc23)C(═O)O
Cc1cc(cc(F)c1NC2=CC(═O)Nc3c(OCC(═O)N[C@@H](Cc4ccc(O)cc4)C(═O)O)c(OC F)ccc23)C(═O)O
CN(CC[N+](C)(C)C)C(═O)CCCOc1c(OC(F)(F)F)ccc2C(═CC(═O)Nc12)Nc3c(C)cc(c c3F)C(═O)O
COc1ccc2C(═CC(═O)Nc2c1OCCCCC(═O)N[C@@H](CC(C)C)C(═O)O)Nc3c(C)cc(c c3F)C(═O)O
Cc1cc(cc(F)c1NC2=CC(═O)Nc3c(OCCCCC(═O)N4CCC[C@H]4C(═O)O)c(OC(F)(F) F)ccc23)C(═O)O
CN(CC[N+](C)(C)C)C(═O)CCCCCOc1c(OC(F)F)ccc2C(═CC(═O)Nc12)Nc3c(C)cc(c c3F)C(═O)O
CN(CC[N+](C)(C)C)C(═O)CCCCCOc1c(OC(F)(F)F)ccc2C(═CC(═O)Nc12)Nc3c(C)c c(cc3F)C(═O)O
CC[C@H](C)[C@H](NC(═O)CCCCCOc1c(OCF)ccc2C(═CC(═O)Nc12)Nc3c(C)cc(cc 3F)C(═O)O)C(═O)O
CCCN(C)CCCCOc1c(OC(F)(F)F)ccc2C(═CC(═O)Nc12)Nc3c(C)cc(cc3Cl)C(═O)O
CCCCCN(C)CCCCOc1c(OC(F)F)ccc2C(═CC(═O)Nc12)Nc3c(C)cc(cc3Cl)C(═O)O
COc1ccc2C(═CC(═O)Nc2c1OCCCCN3CCCC3)Nc4c(C)cc(cc4Cl)C(═O)O
Cc1cc(cc(Cl)c1NC2=CC(═O)Nc3c(OCCCCn4cnnc4)c(OCF)ccc23)C(═O)O
Cc1cc(cc(Cl)c1NC2=CC(═O)Nc3c(OCCCCCn4cccc4)c(OCF)ccc23)C(═O)O
Cc1cc(cc(Cl)c1NC2=CC(═O)Nc3c(OCCCCCn4cncn4)c(OC(F)(F)F)ccc23)C(═O)O
COc1ccc2C(═CC(═O)Nc2c1OCCCCCn3cnnn3)Nc4c(C)cc(cc4Cl)C(═O)O
CN(C)CCCCCCOc1c(OCF)ccc2C(═CC(═O)Nc12)Nc3c(C)cc(cc3Cl)C(═O)O
CCN(C)CCCCCCOc1c(OC(F)(F)F)ccc2C(═CC(═O)Nc12)Nc3c(C)cc(cc3Cl)C(═O)O
COCCOCCCCCCOc1c(OC)ccc2C(═CC(═O)Nc12)Nc3c(C)cc(cc3Cl)C(═O)O
Cc1cc(cc(Cl)c1NC2=CC(═O)Nc3c(OCCCCCCOCCOCCOCCO)c(OC(F)F)ccc23)C(═O)O
CN(CC[N+](C)(C)C)C(═O)COc1c(OC(F)(F)F)ccc2C(═CC(═O)Nc12)Nc3c(C)cc(cc3C 1)C(═O)O
Cc1cc(cc(Cl)c1NC2=CC(═O)Nc3c(OCCCC(═O)N4CCNCC4)c(OC(F)F)ccc23)C(═O) O
Cc1cc(cc(Cl)c1NC2=CC(═O)Nc3c(OCCCCC(═O)NCC(═O)O)c(OC(F)F)ccc23)C(═O) O
Cc1cc(cc(Cl)c1NC2=CC(═O)Nc3c(OCCCCC(═O)N[C@@H](Cc4ccccc4)C(═O)O)c(OCF)ccc23)C(═O)O
CSCC[C@H](NC(═O)CCCCOc1c(OC(F)(F)F)ccc2C(═CC(═O)Nc12)Nc3c(C)cc(cc3C
CN1CCN(CC1)C(═O)CCCCCOc2c(OCF)ccc3C(═CC(═O)Nc23)Nc4c(C)cc(cc4Cl)C(═O)O
COc1ccc2C(═CC(═O)Nc2c1OCCCCCC(═O)N[C@@H](C0)C(═O))Nc3c(C)cc(cc3Cl)C(═O)O
CN(C)CCCCOc1c(OCF)ccc2C(═CC(═O)Nc12)Nc3c(C)cc(cc3Br)C(═O)O
COc1ccc2C(═CC(═O)Nc2c1OCCCCCN3CCOCC3)Nc4c(C)cc(cc4Br)C(═O)O
Cc1cc(cc(Br)c1NC2=CC(═O)Nc3c(OCCCCCOCCOCCO)c(OCF)ccc23)C(═O)O
Cc1cc(cc(Br)c1NC2=CC(═O)Nc3c(OCCCCCCN)c(OC(F)F)ccc23)C(═O)O
COc1ccc2C(═CC(═O)Nc2c1OCCCCCCN3CCCC3)Nc4c(C)cc(cc4Br)C(═O)O
Cc1cc(cc(Br)c1NC2=CC(═O)Nc3c(OCCCCCCn4cnnn4)c(OC(F)(F)F)ccc23)C(═O)O
COc1ccc2C(═CC(═O)Nc2c1OCC(═O)N[C@@H](Cc3c[nH]c4ccccc34)C(═O)O)Nc5c (C)cc(cc5Br)C(═O)O
Cc1cc(cc(Br)c1NC2=CC(═O)Nc3c(OCC(═O)N[C@@H](Cc4c[nH]c5ccccc45)C(═O) O)c(OCF)ccc23)C(═O)O
COc1ccc2C(═CC(═O)Nc2c1OCCCCC(═O)N3CCN(C)CC3)Nc4c(C)cc(cc4Br)C(═O) O
Cc1cc(cc(Br)c1NC2=CC(═O)Nc3c(OCCCCCC(═O)N)c(OC(F)F)ccc23)C(═O)O
COc1ccc2C(═CC(═O)Nc2c1OCCCCCC(═O)NCC(═O))Nc3c(C)cc(cc3Br)C(═O)O
C[C@H](NC(═O)CCCCCOc1c(OCF)ccc2C(═CC(═O)Nc12)Nc3c(C)cc(cc3Br)C(═O) O)C(═O)O
Cc1cc(cc(Br)c1NC2=CC(═O)Nc3c(OCCCCCC(═O)N[C@@H](C0)C(═O)O)c(OC(F) (F)F)ccc23)C(═O)O
CCN(CC)CCCCOc1c(OCF)ccc2C(═CC(═O)Nc12)Nc3c(F)cccc3F
NC(═N)NCCCCOc1c(OC(F)(F)F)ccc2C(═CC(═O)Nc12)Nc3c(F)cccc3F
COc1ccc2C(═CC(═O)Nc2c1OCCCCOCCOCCOCCO)Nc3c(F)cccc3F
FCOc1ccc2C(═CC(═O)Nc2c1OCCCCCN3CCOCC3)Nc4c(F)cccc4F
FC(F)Oc1ccc2C(═CC(═O)Nc2c1OCCCCCn3ccnn3)Nc4c(F)cccc4F
COCCOCCOCCCCCOc1c2NC(═O)C═C(Nc3c(F)cccc3F)c2ccc1OC(F)(F)F
FC(F)Oc1ccc2C(═CC(═O)Nc2c1OCCCCCCn3ccnc3)Nc4c(F)cccc4F
COCCOCCCCCCOc1c(OC)ccc2C(═CC(═O)Nc12)Nc3c(F)cccc3F
NCCCC[C@H](NC(═O)COc1c(OC(F)(F)F)ccc2C(═CC(═O)Nc12)Nc3c(F)cccc3F)C(═O)O
NC(═N)NCCC[C@H](NC(═O)COc1c(OCF)ccc2C(═CC(═O)Nc12)Nc3c(F)cccc3F)C(═O)O
NC(═O)CC[C@H](NC(═O)COc1c(OC(F)F)ccc2C(═CC(═O)Nc12)Nc3c(F)cccc3F)C(═O)O
NC(═O)CCCOc1c(OC(F)F)ccc2C(═CC(═O)Nc12)Nc3c(F)cccc3F
COc1ccc2C(═CC(═O)Nc2c1OCCCC(═O)N3CCNCC3)Nc4c(F)cccc4F
NC(═O)CCCCOc1c(OCF)ccc2C(═CC(═O)Nc12)Nc3c(F)cccc3F
C[N+](C)(C)CCNC(═O)CCCCOc1c(OC(F)F)ccc2C(═CC(═O)Nc12)Nc3c(F)cccc3F
C[N+](C)(C)CCNC(═O)CCCCOc1c(OC(F)(F)F)ccc2C(═CC(═O)Nc12)Nc3c(F)cccc3F
COc1ccc2C(═CC(═O)Nc2c1OCCCCC(═O)N(C)CC[N+](C)(C)C)Nc3c(F)cccc3F
NCCCC[C@H](NC(═O)CCCCCOc1c(OCF)ccc2C(═CC(═O)Nc12)Nc3c(F)cccc3F)C(═O)O
COc1ccc2C(═CC(═O)Nc2c1OCCCCC(═O)N[C@@H](Cc3cnc[nH]3)C(═O)O)Nc4c(F)cccc4F
NCCCCOc1c(OCF)ccc2C(═CC(═O)Nc12)Nc3c(F)cccc3Cl
Fc1cccc(Cl)c1NC2=CC(═O)Nc3c(OCCCCn4nccn4)c(OC(F)(F)F)ccc23
COCCOCCOCCOCCCCOc1c(OC(F)F)ccc2C(═CC(═O)Nc12)Nc3c(F)cccc3Cl
COc1ccc2C(═CC(═O)Nc2c1OCCCCCN(C)C)Nc3c(F)cccc3Cl
Fc1cccc(Cl)c1NC2=CC(═O)Nc3c(OCCCCCN4CCCC(F)(F)C4)c(OC(F)(F)F)ccc23
FCOc1ccc2C(═CC(═O)Nc2c1OCCCCCOc3ccccn3)Nc4c(F)cccc4Cl
OCCOCCOCCCCCCOc1c(OC(F)(F)F)ccc2C(═CC(═O)Nc12)Nc3c(F)cccc3Cl
CN(CCC[N+](C)(C)C)C(═O)COc1c(OCF)ccc2C(═CC(═O)Nc12)Nc3c(F)cccc3Cl
CN1CCN(CC1)C(═O)COc2c(OC(F)(F)F)ccc3C(═CC(═O)Nc23)Nc4c(F)cccc4Cl
COc1ccc2C(═CC(═O)Nc2c1OCC(═O)N[C@@H](CCCCN)C(═O))Nc3c(F)cccc3Cl
COc1ccc2C(═CC(═O)Nc2c1OCCCCC(═O)N[C@@H](C)C(═O))Nc3c(F)cccc3C1
C(═O)[C@H](Cc1ccc(O)cc1)NC(═O)CCCOc2c(OCF)ccc3C(═CC(═O)Nc23)Nc4c(F)cccc4Cl
C[C@@H](O)[C@H](NC(═O)CCCOc1c(OC(F)F)ccc2C(═CC(═O)Nc12)Nc3c(F)cccc 3Cl)C(═O)O
CN(CC[N+](C)(C)C)C(═O)CCCCOc1c(OCF)ccc2C(═CC(═O)Nc12)Nc3c(F)cccc3Cl
OC(═O)CNC(═O)CCCCOc1c(OC(F)F)ccc2C(═CC(═O)Nc12)Nc3c(F)cccc3Cl
C[C@@H](O)[C@H](NC(═O)CCCCOc1c(OC(F)(F)F)ccc2C(═CC(═O)Nc12)Nc3c(F) cccc3Cl)C(═O)O
OC(═O)[C@H](Cc1cnc[nH]1)NC(═O)CCCCCOc2c(OC(F)(F)F)ccc3C(═CC(═O)Nc23)Nc4c(F)cccc4Cl
NC(═O)C[C@H](NC(═O)CCCCCOc1c(OC(F)F)ccc2C(═CC(═O)Nc12)Nc3c(F)cccc3Cl)C(═O)O
COc1ccc2C(═CC(═O)Nc2c1OCCCCOc3cccnc3)Nc4c(F)cccc4Br
FC(F)Oc1ccc2C(═CC(═O)Nc2c1OCCCCCN3CCC(F)CC3)Nc4c(F)cccc4Br
FCOc1ccc2C(═CC(═O)Nc2c1OCCCCCOc3ccccn3)Nc4c(F)cccc4Br
CNCCCCCCOc1c(OC(F)F)ccc2C(═CC(═O)Nc12)Nc3c(F)cccc3Br
COCCN(C)CCCCCCOc1c(OC(F)(F)F)ccc2C(═CC(═O)Nc12)Nc3c(F)cccc3Br
FCOc1ccc2C(═CC(═O)Nc2c1OCCCCCN3CCC(F)(F)C3)Nc4c(F)cccc4Br
NC(═O)COc1c(OC(F)F)ccc2C(═CC(═O)Nc12)Nc3c(F)cccc3Br
CC[C@H](C)[C@H](NC(═O)COc1c(OCF)ccc2C(═CC(═O)Nc12)Nc3c(F)cccc3Br)C(═O)O
NC(═O)CCCOc1c(OC(F)(F)F)ccc2C(═CC(═O)Nc12)Nc3c(F)cccc3Br
CN(C)CCNC(═O)CCCOc1c(OCF)ccc2C(═CC(═O)Nc12)Nc3c(F)cccc3Br
OC(═O)[C@H](Cc1ccccc1)NC(═O)CCCCOc2c(OCF)ccc3C(═CC(═O)Nc23)Nc4c(F)c ccc4Br
COc1ccc2C(═CC(═O)Nc2c1OCCCCC(═O)N[C@@H](CO)C(═O))Nc3c(F)cccc3Br
CN(C)CCNC(═O)CCCCCOc1c(OC(F)F)ccc2C(═CC(═O)Nc12)Nc3c(F)cccc3Br
OC(═O)[C@H](Cc1c[nH]c2ccccc12)NC(═O)CCCCCOc3c(OCF)ccc4C(═CC(═O)Nc3 4)Nc5c(F)cccc5Br
CCCN(C)CCCCOc1c(OC(F)F)ccc2C(═CC(═O)Nc12)Nc3c(F)cc(F)cc3F
Fc1cc(F)c(NC2=CC(═O)Nc3c(OCCCCn4ccnc4)c(OC(F)(F)F)ccc23)c(F)c1
CCCNCCCCCOc1c(OC)ccc2C(═CC(═O)Nc12)Nc3c(F)cc(F)cc3F
NC(═N)NCCCCCOc1c(OCF)ccc2C(═CC(═O)Nc12)Nc3c(F)cc(F)cc3F
NC(═N)NCCCCCOc1c(OC(F)F)ccc2C(═CC(═O)Nc12)Nc3c(F)cc(F)cc3F
CCCNCCCCCCOc1c(OC(F)(F)F)ccc2C(═CC(═O)Nc12)Nc3c(F)cc(F)cc3F
FC(F)Oc1ccc2C(═CC(═O)Nc2c1OCCCCCCN3CCCC(F)C3)Nc4c(F)cc(F)cc4F
COc1ccc2C(═CC(═O)Nc2c1OCCCCCCn3ccnc3)Nc4c(F)cc(F)cc4F
CN(C)CCOC(═O)COc1c(OCF)ccc2C(═CC(═O)Nc12)Nc3c(F)cc(F)cc3F
COc1ccc2C(═CC(═O)Nc2c1OCC(═O)NCC[N+](C)(C)C)Nc3c(F)cc(F)cc3F
CN(C)CCCN(C)C(═O)COc1c(OC(F)F)ccc2C(═CC(═O)Nc12)Nc3c(F)cc(F)cc3F
CN(C)CCNC(═O)CCCOc1c(OCF)ccc2C(═CC(═O)Nc12)Nc3c(F)cc(F)cc3F
COc1ccc2C(═CC(═O)Nc2c1OCCCC(═O)N[C@@H](CC(C)C)C(═O)O)Nc3c(F)cc(F) cc3F
C[N+](C)(C)CCOC(═O)CCCCOc1c(OC(F)F)ccc2C(═CC(═O)Nc12)Nc3c(F)cc(F)cc3F
COc1ccc2C(═CC(═O)Nc2c1OCCCCC(═O)N[C@@H](Cc3ccc(O)cc3)C(═O)O)Nc4c(F)cc(F)cc4F
NC(═O)C[C@H](NC(═O)CCCCOc1c(OC(F)(F)F)ccc2C(═CC(═O)Nc12)Nc3c(F)cc(F)cc3F)C(═O)O
C[N+](C)(C)CCOC(═O)CCCCCOc1c(OCF)ccc2C(═CC(═O)Nc12)Nc3c(F)cc(F)cc3F
OC(═O)[C@H](Cc1c[nH]c2ccccc12)NC(═O)CCCCCOc3c(OC(F)F)ccc4C(═CC(═O) Nc34)Nc5c(F)cc(F)cc5F
NC(═N)NCCC[C@H](NC(═O)CCCCCOc1c(OC(F)(F)F)ccc2C(═CC(═O)Nc12)Nc3c(F)cc(F)cc3F)C(═O)O
CCCN(C)CCCCOc1c(OCF)ccc2C(═CC(═O)Nc12)Nc3c(F)cc(F)cc3Cl
FC(F)Oc1ccc2C(═CC(═O)Nc2c1OCCCCN3CCCCC3)Nc4c(F)cc(F)cc4Cl
COc1ccc2C(═CC(═O)Nc2c1OCCCCN3CCCC(F)C3)Nc4c(F)cc(F)cc4Cl
CCN(C)CCCCCOc1c(OCF)ccc2C(═CC(═O)Nc12)Nc3c(F)cc(F)cc3Cl
COc1ccc2C(═CC(═O)Nc2c1OCCCCCN3CCC(F)(F)CC3)Nc4c(F)cc(F)cc4Cl
COCCOCCOCCCCCOc1c(OC(F)(F)F)ccc2C(═CC(═O)Nc12)Nc3c(F)cc(F)cc3Cl
Fc1cc(F)c(NC2=CC(═O)Nc3c(OCCCCCCN4CCCCC4)c(OC(F)(F)F)ccc23)c(Cl)c1
COc1ccc2C(═CC(═O)Nc2c1OCCCCCCN3CCCC3)Nc4c(F)cc(F)cc4Cl
NC(═O)COc1c(OC(F)F)ccc2C(═CC(═O)Nc12)Nc3c(F)cc(F)cc3Cl
COc1ccc2C(═CC(═O)Nc2c1OCC(═O)N(C)CCN(C)C)Nc3c(F)cc(F)cc3Cl
CN(C)CCN(C)C(═O)COc1c(OC(F)(F)F)ccc2C(═CC(═O)Nc12)Nc3c(F)cc(F)cc3Cl
C[N+](C)(C)CCNC(═O)CCCOc1c(OC(F)F)ccc2C(═CC(═O)Nc12)Nc3c(F)cc(F)cc3Cl
NC(═O)CCCCOc1c(OC(F)(F)F)ccc2C(═CC(═O)Nc12)Nc3c(F)cc(F)cc3Cl
COc1ccc2C(═CC(═O)Nc2c1OCCCCC(═O)N(C)CCC[N+](C)(C)C)Nc3c(F)cc(F)cc3Cl
C[C@H](NC(═O)CCCCOc1c(OC(F)F)ccc2C(═CC(═O)Nc12)Nc3c(F)cc(F)cc3Cl)C(═O)O
CN(C)CCOC(═O)CCCCCOc1c(OC(F)F)ccc2C(═CC(═O)Nc12)Nc3c(F)cc(F)cc3Cl
CN(CCC[N+](C)(C)C)C(═O)CCCCCOc1c(OCF)ccc2C(═CC(═O)Nc12)Nc3c(F)cc(F)c c3Cl
OC(═O)[C@H](Cc1c[nH]c2ccccc12)NC(═O)CCCCCOc3c(OC(F)(F)F)ccc4C(═CC(═O)Nc34)Nc5c(F)cc(F)cc5Cl
FC(F)Oc1ccc2C(═CC(═O)Nc2c1OCCCCN3CCCC3)Nc4c(F)cc(F)cc4Br
FCOc1ccc2C(═CC(═O)Nc2c1OCCCCOc3ccccn3)Nc4c(F)cc(F)cc4Br
CCNCCCCCOc1c(OC(F)F)ccc2C(═CC(═O)Nc12)Nc3c(F)cc(F)cc3Br
FCOc1ccc2C(═CC(═O)Nc2c1OCCCCCCOc3cccnc3)Nc4c(F)cc(F)cc4Br
OC[C@H]1O[C@@H](OCCCCCCOc2c(OC(F)F)ccc3C(═CC(═O)Nc23)Nc4c(F)cc(F)cc4Br)[C@H](O)[C@@H](O)[C@H]1O
COCCOCCOCCCCCCOc1c(OC)ccc2C(═CC(═O)Nc12)Nc3c(F)cc(F)cc3Br
OCCOCCOCCOCCCCCCOc1c(OC(F)(F)F)ccc2C(═CC(═O)Nc12)Nc3c(F)cc(F)cc3Br
COc1ccc2C(═CC(═O)Nc2c1OCC(═O)N[C@@H]([C@@H](C)O)C(═O))Nc3c(F)cc(F)cc3Br
COc1ccc2C(═CC(═O)Nc2c1OCCCC(═O)N[C@@H](CCSC)C(═O)O)Nc3c(F)cc(F)cc 3Br
NC(═N)NCCC[C@H](NC(═O)CCCOc1c(OC(F)F)ccc2C(═CC(═O)Nc12)Nc3c(F)cc(F)cc3Br)C(═O)O
CN(C)CCNC(═O)CCCCOc1c(OCF)ccc2C(═CC(═O)Nc12)Nc3c(F)cc(F)cc3Br
CN(CCC[N+](C)(C)C)C(═O)CCCCOc1c(OC(F)F)ccc2C(═CC(═O)Nc12)Nc3c(F)cc(F)cc3Br
CN(C)CCCN(C)C(═O)CCCCCOc1c(OC(F)(F)F)ccc2C(═CC(═O)Nc12)Nc3c(F)cc(F)c c3Br
OC(═O)[C@H](Cc1c[nH]c2ccccc12)NC(═O)CCCCCOc3c(OCF)ccc4C(═CC(═O)Nc3 4)Nc5c(F)cc(F)cc5Br
COc1ccc2C(═CC(═O)Nc2c1OCCCCCC(═O)N[C@@H](CCCCN)C(═O)O)Nc3c(F)cc (F)cc3Br
Oc1cc(F)c(NC2=CC(═O)Nc3c(OCCCCN4CCCC(F)(F)C4)c(OC(F)(F)F)ccc23)c(F)c1
COc1ccc2C(═CC(═O)Nc2c1OCCCCOCCO)Nc3c(F)cc(O)cc3F
COCCOCCOCCOCCCCOc1c(OC(F)F)ccc2C(═CC(═O)Nc12)Nc3c(F)cc(O)cc3F
CN(C)CCCCCOc1c(OCF)ccc2C(═CC(═O)Nc12)Nc3c(F)cc(O)cc3F
COc1ccc2C(═CC(═O)Nc2c1OCCCCCN3CCCC(F)C3)Nc4c(F)cc(O)cc4F
Oc1cc(F)c(NC2=CC(═O)Nc3c(OCCCCCN4CCCC(F)C4)c(OC(F)(F)F)ccc23)c(F)c1
Oc1cc(F)c(NC2=CC(═O)Nc3c(OCCCCCn4ccnc4)c(OC(F)F)ccc23)c(F)c1
Oc1cc(F)c(NC2=CC(═O)Nc3c(OCCCCCCN4CCC(F)(F)CC4)c(OC(F)F)ccc23)c(F)c1
COc1ccc2C(═CC(═O)Nc2c1OCCCCCCOc3ccccn3)Nc4c(F)cc(O)cc4F
C[N+](C)(C)CCOC(═O)COc1c(OCF)ccc2C(═CC(═O)Nc12)Nc3c(F)cc(O)cc3F
COc1ccc2C(═CC(═O)Nc2c1OCC(═O)N[C@@H](Cc3ccccc3)C(═O)O)Nc4c(F)cc(O)c c4F
C[N+](C)(C)CCNC(═O)CCCOc1c(OCF)ccc2C(═CC(═O)Nc12)Nc3c(F)cc(O)cc3F
C[N+](C)(C)CCNC(═O)CCCOc1c(OC(F)F)ccc2C(═CC(═O)Nc12)Nc3c(F)cc(O)cc3F
CN(CC[N+](C)(C)C)C(═O)CCCOc1c(OC(F)(F)F)ccc2C(═CC(═O)Nc12)Nc3c(F)cc(O)cc3F
COc1ccc2C(═CC(═O)Nc2c1OCCCCC(═O)OCCN(C)C)Nc3c(F)cc(O)cc3F
C[N+](C)(C)CCNC(═O)CCCCOc1c(OC(F)(F)F)ccc2C(═CC(═O)Nc12)Nc3c(F)cc(O)c c3F
CN(C)CCOC(═O)CCCCCOc1c(OC(F)(F)F)ccc2C(═CC(═OCC(═O)Nc12)Nc3c(F)cc(O)cc3F
COc1ccc2C(═CC(═O)Nc2c1OCCCCCC(═O)N[C@@H](CC(═O)N)C(═O)O)Nc3c(F)c c(O)cc3F
NC(═O)CC[C@H](NC(═O)CCCCCOc1c(OCF)ccc2C(═CC(═O)Nc12)Nc3c(F)cc(O)cc 3F)C(═O)O
COc1ccc2C(═CC(═O)Nc2c1OCCCCN)Nc3c(F)cc(O)cc3Cl
Oc1cc(F)c(NC2=CC(═O)Nc3c(OCCCCn4cccc4)c(OC(F)(F)F)ccc23)c(Cl)c1
Oc1cc(F)c(NC2=CC(═O)Nc3c(OCCCCCn4cnnc4)c(OC(F)F)ccc23)c(Cl)c1
COc1ccc2C(═CC(═O)Nc2c1OCCCCCOc3cccnc3)Nc4c(F)cc(O)cc4Cl
OCCOCCOCCOCCCCCOc1c(OCF)ccc2C(═CC(═O)Nc12)Nc3c(F)cc(O)cc3Cl
CCCCCN(C)CCCCCCOc1c(OC)ccc2C(═CC(═O)Nc12)Nc3c(F)cc(O)cc3Cl
Oc1cc(F)c(NC2=CC(═O)Nc3c(OCCCCCCn4ncnn4)c(OC(F)(F)F)ccc23)c(Cl)c1
CN(C)CCOC(═O)COc1c(OCF)ccc2C(═CC(═O)Nc12)Nc3c(F)cc(O)cc3Cl
NC(═N)NCCC[C@H](NC(═O)COc1c(OC(F)(F)F)ccc2C(═CC(═O)Nc12)Nc3c(F)cc(O)cc3Cl)C(═O)O
CSCC[C@H](NC(═O)CCCOc1c(OC(F)(F)F)ccc2C(═CC(═O)Nc12)Nc3c(F)cc(O)cc3Cl)C(═O)O
COc1ccc2C(═CC(═O)Nc2c1OCCCC(═O)N[C@@H](CO)C(═O)O)Nc3c(F)cc(O)cc3C 1
C(═O)[C@H](Cc1cnc[nH]1)NC(═O)CCCOc2c(OCF)ccc3C(═CC(═O)Nc23)Nc4c(F) cc(O)cc4Cl
CN(C)CCCN(C)C(═O)CCCCOc1c(OC(F)(F)F)ccc2C(═CC(═O)Nc12)Nc3c(F)cc(O)cc 3Cl
COc1ccc2C(═CC(═O)Nc2c1OCCCCCC(═O)N[C@@H](Cc3c[nH]c4ccccc34)C(═O)O) Nc5c(F)cc(O)cc5Cl
COc1ccc2C(═CC(═O)Nc2c1OCCCCCC(═O)N[C@@H](CCSC)C(═O)O)Nc3c(F)cc(O)cc3Cl
C[C@@H](O)[C@H](NC(═O)CCCCCOc1c(OC(F)(F)F)ccc2C(═CC(═O)Nc12)Nc3c(F)cc(O)cc3Cl)C(═O)O
NC(═N)NCCC[C@H](NC(═O)CCCCCOc1c(OCF)ccc2C(═CC(═O)Nc12)Nc3c(F)cc(O)cc3Cl)C(═O)O
CCNCCCCOc1c(OC(F)F)ccc2C(═CC(═O)Nc12)Nc3c(F)cc(O)cc3Br
NC(═N)NCCCCOc1c(OC(F)(F)F)ccc2C(═CC(═O)Nc12)Nc3c(F)cc(O)cc3Br
Oc1cc(F)c(NC2=CC(═O)Nc3c(OCCCCn4cnnn4)c(OCF)ccc23)c(Br)c1
COc1ccc2C(═CC(═O)Nc2c1OCCCCCn3nccn3)Nc4c(F)cc(O)cc4Br
OCCOCCOCCCCCOc1c(OCF)ccc2C(═CC(═O)Nc12)Nc3c(F)cc(O)cc3Br
COc1ccc2C(═CC(═O)Nc2c1OCCCCCCN3CCC(F)C3)Nc4c(F)cc(O)cc4Br
C[N+](C)(C)CCOC(═O)COc1c(OC(F)(F)F)ccc2C(═CC(═O)Nc12)Nc3c(F)cc(O)cc3Br
OC(═O)[C@H](Cc1ccccc1)NC(═O)COc2c(OCF)ccc3C(═CC(═O)Nc23)Nc4c(F)cc(O)cc4Br
COc1ccc2C(═CC(═O)Nc2c1OCC(═O)N[C@@H](Cc3ccc(O)cc3)C(═O)O)Nc4c(F)cc(O)cc4Br
OC(═O)CC[C@H](NC(═O)COc1c(OC(F)F)ccc2C(═CC(═O)Nc12)Nc3c(F)cc(O)cc3B r)C(═O)O
NCCCC[C@H](NC(═O)CCCOc1c(OC(F)F)ccc2C(═CC(═O)Nc12)Nc3c(F)cc(O)cc3Br)C(═O)O
OC(═O)CC[C@H](NC(═O)CCCOc1c(OC(F)(F)F)ccc2C(═CC(═O)Nc12)Nc3c(F)cc(O)cc3Br)C(═O)O
CN(CC[N+](C)(C)C)C(═O)CCCCOc1c(OCF)ccc2C(═CC(═O)Nc12)Nc3c(F)cc(O)cc3Br
COc1ccc2C(═CC(═O)Nc2c1OCCCCC(═O)N[C@@H](CCCNC(═N)N)C(═O)O)Nc3c (F)cc(O)cc3Br
COc1ccc2C(═CC(═O)Nc2c1OCCCCCC(═O)N)Nc3c(F)cc(O)cc3Br
Oc1cc(F)c(NC2=CC(═O)Nc3c(OCCCCCC(═O)N4CCNCC4)c(OCF)ccc23)c(Br)c1
CN1CCN(CC1)C(═O)CCCCCOc2c(OC(F)(F)F)ccc3C(═CC(═O)Nc23)Nc4c(F)cc(O)c c4Br
CSCC[C@H](NC(═O)CCCCCOc1c(OC(F)F)ccc2C(═CC(═O)Nc12)Nc3c(F)cc(O)cc3Br)C(═O)O
CCCCN(C)CCCCOc1c(OC(F)(F)F)ccc2C(═CC(═O)Nc12)Nc3c(F)cc(cc3F)C(═O)O
COc1ccc2C(═CC(═O)Nc2c1OCCCCOCCOCCO)Nc3c(F)cc(cc3F)C(═O)O
COCCOCCOCCOCCCCOc1c(OCF)ccc2C(═CC(═O)Nc12)Nc3c(F)cc(cc3F)C(═O)O
OC(═O)c1cc(F)c(NC2=CC(═O)Nc3c(OCCCCCn4ccnc4)c(OC(F)F)ccc23)c(F)c1
C(═O)c1cc(F)c(NC2=CC(═O)Nc3c(OCCCCCn4cnnc4)c(OCF)ccc23)c(F)c1
COCCOCCOCCOCCCCCOc1c(OC)ccc2C(═CC(═O)Nc12)Nc3c(F)cc(cc3F)C(═O)O
COc1ccc2C(═CC(═O)Nc2c1OCCCCCCN3CCOCC3)Nc4c(F)cc(cc4F)C(═O)O
OC(═O)c1cc(F)c(NC2=CC(═O)Nc3c(OCCCCCCN4CCOCC4)c(OC(F)F)ccc23)c(F)c1
COc1ccc2C(═CC(═O)Nc2c1OCC(═O)NCC[N+](C)(C)C)Nc3c(F)cc(cc3F)C(═O)O
CN(C)CCN(C)C(═O)COc1c(OCF)ccc2C(═CC(═O)Nc12)Nc3c(F)cc(cc3F)C(═O)O
CN(CCC[N+](C)(C)C)C(═O)CCCOc1c(OC(F)(F)F)ccc2C(═CC(═O)Nc12)Nc3c(F)cc(cc3F)C(═O)O
OC(═O)c1cc(F)c(NC2=CC(═O)Nc3c(OCCCC(═O)N4CCNCC4)c(OC(F)F)ccc23)c(F) c1
COc1ccc2C(═CC(═O)Nc2c1OCCCC(═O)N[C@@H](CO)C(═O)O)Nc3c(F)cc(cc3F)C (═O)O
OC(═O)[C@@H]1CCCN1C(═O)CCCCOc2c(OC(F)F)ccc3C(═CC(═O)Nc23)Nc4c(F) cc(cc4F)C(═O)O
C[C@@H](O)[C@H](NC(═O)CCCCOc1c(OC(F)(F)F)ccc2C(═CC(═O)Nc12)Nc3c(F) cc(cc3F)C(═O)O)C(═O)O
COc1ccc2C(═CC(═O)Nc2c1OCCCCCC(═O)N[C@@H](CC(C)C)C(═O)O)Nc3c(F)cc (cc3F)C(═O)O
CSCC[C@H](NC(═O)CCCCCOc1c(OC(F)F)ccc2C(═CC(═O)Nc12)Nc3c(F)cc(cc3F)
CSCC[C@H](NC(═O)CCCCCOc1c(OC(F)(F)F)ccc2C(═CC(═O)Nc12)Nc3c(F)cc(cc3
COc1ccc2C(═CC(═O)Nc2c1OCCCCn3nccn3)Nc4c(F)cc(cc4Cl)C(═O)O
OCCOCCCCOc1c(OC(F)(F)F)ccc2C(═CC(═O)Nc12)Nc3c(F)cc(cc3Cl)C(═O)O
OC(═O)c1cc(F)c(NC2=CC(═O)Nc3c(OCCCCCN4CCCC(F)(F)CC4)c(OC(F)(F)F)ccc23)(C)c1
COc1ccc2C(═CC(═O)Nc2c1OCCCCCn3cccn3)Nc4c(F)cc(cc4Cl)C(═O)O
OC(═O)c1cc(F)c(NC2=CC(═O)Nc3c(OCCCCCOc4ccncc4)c(OCF)ccc23)c(Cl)c1
C(═O)c1cc(F)c(NC2=CC(═O)Nc3c(OCCCCCCN4CCCC(F)(F)C4)c(OC(F)(F)F)ccc 23)c(Cl)c1
C(═O)c1cc(F)c(NC2=CC(═O)Nc3c(OCCCCCCn4ncnn4)c(OC(F)F)ccc23)c(Cl)c1
COCCOCCOCCCCCCOc1c(OCF)ccc2C(═CC(═O)Nc12)Nc3c(F)cc(cc3Cl)C(═O)O
COc1ccc2C(═CC(═O)Nc2c1OCC(═O)N3CCNCC3)Nc4c(F)cc(cc4Cl)C(═O)O
CSCC[C@H](NC(═O)COc1c(OC(F)F)ccc2C(═CC(═O)Nc12)Nc3c(F)cc(cc3Cl)C(═O) O)C(═O)O
COc1ccc2C(═CC(═O)Nc2c1OCCCC(═O)NCC(═O)O)Nc3c(F)cc(cc3Cl)C(═O)O
C[C@@H](O)[C@H](NC(═O)CCCOc1c(OCF)ccc2C(═CC(═O)Nc12)Nc3c(F)cc(cc3Cl)C(═O)O)C(═O)O
NC(═N)NCCC[C@H](NC(═O)CCCOc1c(OC(F)(F)F)ccc2C(═CC(═O)Nc12)Nc3c(F)c c(cc3Cl)C(═O)O)C(═O)O
OC(═O)[C@H](Cc1cnc[nH]1)NC(═O)CCCOc2c(OC(F)F)ccc3C(═CC(═O)Nc23)Nc4c (F)cc(cc4Cl)C(═O)O
C[N+](C)(C)CCNC(═O)CCCCOc1c(OC(F)F)ccc2C(═CC(═O)Nc12)Nc3c(F)cc(cc3Cl) C(═O)O
CN(C)CCCN(C)C(═O)CCCCOc1c(OC(F)(F)F)ccc2C(═CC(═O)Nc12)Nc3c(F)cc(cc3C 1)C(═O)O
CCCN(C)CCCCOc1c(OC(F)(F)F)ccc2C(═CC(═O)Nc12)Nc3c(F)cc(cc3Br)C(═O)O
COc1ccc2C(═CC(═O)Nc2c1OCCCCN3CCOCC3)Nc4c(F)cc(cc4Br)C(═O)O
OC(═O)c1cc(F)c(NC2=CC(═O)Nc3c(OCCCCn4cccc4)c(OC(F)F)ccc23)c(Br)c1
CCN(CC)CCCCCOc1c(OCF)ccc2C(═CC(═O)Nc12)Nc3c(F)cc(cc3Br)C(═O)O
OC(═O)c1cc(F)c(NC2=CC(═O)Nc3c(OCCCCCn4ccnn4)c(OC(F)F)ccc23)c(Br)c1
C(═O)c1cc(F)c(NC2=CC(═O)Nc3c(OCCCCCn4cncn4)c(OC(F)(F)F)ccc23)c(Br)c1
C(═O)c1cc(F)c(NC2=CC(═O)Nc3c(OCCCCCCn4ccnn4)c(OC(F)(F)F)ccc23)c(Br)c1
COc1ccc2C(═CC(═O)Nc2c1OCCCCCCOCCOCCO)Nc3c(F)cc(cc3Br)C(═O)O
CC(C)C[C@H](NC(═O)COc1c(OCF)ccc2C(═CC(═O)Nc12)Nc3c(F)cc(cc3Br)C(═O) O)C(═O)O
COc1ccc2C(═CC(═O)Nc2c1OCC(═O)N[C@@H](Cc3ccc(O)cc3)C(═O)O)Nc4c(F)cc(cc4Br)C(═O)O
NC(═O)C[C@H](NC(═O)COc1c(OC(F)F)ccc2C(═CC(═O)Nc12)Nc3c(F)cc(cc3Br)C(═O)OC(═O)O
CC(C)[C@H](NC(═O)CCCOc1c(OC)(F)(F)F)ccc2C(═CC(═O)Nc12Nc3c(F)cc(cc3Br)
NCCCC[C@H](NC(═O)CCCOc1c(OC(F)F)ccc2C(═CC(═O)Nc12)Nc3c(F)cc(cc3Br)
COc1ccc2C(═CC(═O)Nc2c1OCCCC(═O)N[C@@H](CC(═O)N)C(═O)O)Nc3c(F)cc(c c3Br)C(═O)O
COc1ccc2C(═CC(═O)Nc2c1OCCCCC(═O)N3CCNCC3)Nc4c(F)cc(cc4Br)C(═O)O
CC(C)C[C@H](NC(═O)CCCCOc1c(OC(F)F)ccc2C(═CC(═O)Nc12)Nc3c(F)cc(cc3Br) C(═O)O)C(═O)O
CC[C@H](C)[C@H](NC(═O)CCCCOc1c(OC(F)(F)F)ccc2C(═CC(═O)Nc12)Nc3c(F)c c(cc3Br)C(═O)O)C(═O)O
CN(C)CCOC(═O)CCCCCOc1c(OC(F)(F)F)ccc2C(═CC(═O)Nc12)Nc3c(F)cc(cc3Br)C (═O)O
C[C@@H](O)[C@H](NC(═O)CCCCCOc1c(OCF)ccc2C(═CC(═O)Nc12)Nc3c(F)cc(c c3Br)C(═O)O)C(═O)O
COc1ccc2C(═CC(═O)Nc2c1OCCCCCC(═O)N[C@@H](CC(═O)N)C(═O)O)Nc3c(F)c c(cc3Br)C(═O)O
CCCNCCCCOc1c(OC)ccc2C(═CC(═O)Nc12)Nc3c(Cl)cccc3Cl
CCCNCCCCOc1c(OC(F)F)ccc2C(═CC(═O)Nc12)Nc3c(Cl)cccc3Cl
CCCNCCCCOc1c(OC(F)(F)F)ccc2C(═CC(═O)Nc12)Nc3c(Cl)cccc3Cl
OCCOCCOCCOCCCCOc1c(OCF)ccc2C(═CC(═O)Nc12)Nc3c(Cl)cccc3Cl
COc1ccc2C(═CC(═O)Nc2c1OCCCCCNC(═N)N)Nc3c(Cl)cccc3Cl
FC(F)(F)Oc1ccc2C(═CC(═O)Nc2c1OCCCCCN3CCOCC3)Nc4c(Cl)cccc4Cl
FCOc1ccc2C(═CC(═O)Nc2c1OCCCCCn3cccc3)Nc4c(Cl)cccc4Cl
CCCCN(C)CCCCCCOc1c(OC(F)F)ccc2C(═CC(═O)Nc12)Nc3c(Cl)cccc3Cl
FCOc1ccc2C(═CC(═O)Nc2c1OCCCCCCOc3cccnc3)Nc4c(Cl)cccc4Cl
CN(C)CCOC(═O)COc1c(OCF)ccc2C(═CC(═O)Nc12)Nc3c(Cl)cccc3Cl
COc1ccc2C(═CC(═O)Nc2c1OCC(═O)N(C)CCCN(C)C)Nc3c(Cl)cccc3C1
C(═O)[C@H](Cc1ccccc1)NC(═O)COc2c(OC(F)F)ccc3C(═CC(═O)Nc23)Nc4c(Cl)cc cc4Cl
NC(═O)CC[C@H](NC(═O)COc1c(OC(F)(F)F)ccc2C(═CC(═O)Nc12)Nc3c(Cl)cccc3C 1)C(═O)O
C[C@@H](O)[C@H](NC(═O)CCCOc1c(OC(F)(F)F)ccc2C(═CC(═O)Nc12)Nc3c(Cl) cccc3Cl)C(═O)O
C[C@H](NC(═O)CCCCOc1c(OC(F)F)ccc2C(═CC(═O)Nc12)Nc3c(Cl)cccc3Cl)C(═O) O
CC[C@H](C)[C@H](NC(═O)CCCCCOc1c(OC(F)F)ccc2C(═CC(═O)Nc12)Nc3c(Cl)c ccc3Cl)C(═O)O
OC(═O)[C@H](Cc1ccc(O)cc1)NC(═O)CCCCCOc2c(OCF)ccc3C(═CC(═O)Nc23)Nc4c(Cl)cccc4Cl
NCCCC[C@H](NC(═O)CCCCCOc1c(OC(F)(F)F)ccc2C(═CC(═O)Nc12)Nc3c(Cl)ccc c3Cl)C(═O)O
COc1ccc2C(═CC(═O)Nc2c1OCCCCCC(═O)N[C@@H](CCC(═O)O)C(═O))Nc3c(C 1)cccc3Cl
NC(═N)NCCCCOc1c(OC(F)F)ccc2C(═CC(═O)Nc12)Nc3c(Cl)cccc3Br
FC1CCCN(CCCCOc2c(OC(F)(F)F)ccc3C(═CC(═O)Nc23)Nc4c(Cl)cccc4Br)C1
COc1ccc2C(═CC(═O)Nc2c1OCCCCn3ccnc3)Nc4c(Cl)cccc4Br
FCOc1ccc2C(═CC(═O)Nc2c1OCCCCCN3CCC(F)CC3)Nc4c(Cl)cccc4Br
COc1ccc2C(═CC(═O)Nc2c1OCCCCCn3cnnn3)Nc4c(Cl)cccc4Br
FC(F)(F)Oc1ccc2C(═CC(═O)Nc2c1OCCCCCn3cnnn3)Nc4c(Cl)cccc4Br
CNCCCCCCOc1c(OC(F)(F)F)ccc2C(═CC(═O)Nc12)Nc3c(Cl)cccc3Br
CCCCN(C)CCCCCCOc1c(OC(F)F)ccc2C(═CC(═O)Nc12)Nc3c(Cl)cccc3Br
FCOc1ccc2C(═CC(═O)Nc2c1OCCCCCCn3cnnc3)Nc4c(Cl)cccc4Br
COCCOCCCCCCOc1c(OC)ccc2C(═CC(═O)Nc12)Nc3c(Cl)cccc3Br
C[N+](C)(C)CCNC(═O)COc1c(OCF)ccc2C(═CC(═O)Nc12)Nc3c(Cl)cccc3Br
COc1ccc2C(═CC(═O)Nc2c1OCC(═O)NCC(═O)O)Nc3c(Cl)cccc3Br
NC(═O)CC[C@H](NC(═O)COc1c(OC(F)F)ccc2C(═CC(═O)Nc12)Nc3c(Cl)cccc3Br) C(═O)O
CN(C)CCNC(═O)CCCOc1c(OCF)ccc2C(═CC(═O)Nc12)Nc3c(Cl)cccc3Br
CC(C)C[C@H](NC(═O)CCCOc1c(OC(F)F)ccc2C(═CC(═O)Nc12)Nc3c(Cl)cccc3Br) C(═O)O
COc1ccc2C(═CC(═O)Nc2c1OCCCCCC(═O)N[C@@H](C)C(═O))Nc3c(Cl)cccc3Br
C[C@H](NC(═O)CCCCOc1c(OC(F)F)ccc2C(═CC(═O)Nc12)Nc3c(Cl)cccc3Br)C(═O) O
OC(═O)C[C@H](NC(═O)CCCCOc1c(OCF)ccc2C(═CC(═O)Nc12)Nc3c(Cl)cccc3Br) C(═O)O
CN(CC[N+](C)(C)C)C(═O)CCCCCOc1c(OC(F)(F)F)ccc2C(═CC(═O)Nc12)Nc3c(Cl) cccc3Br
COCCOCCOCCCCOc1c(OCF)ccc2C(═CC(═O)Nc12)Nc3c(Cl)cc(F)cc3Cl
COc1ccc2C(═CC(═O)Nc2c1OCCCCOCCOCCOCCO)Nc3c(Cl)cc(F)cc3Cl
Fc1cc(Cl)c(NC2=CC(═O)Nc3c(OCCCCCN4CCC(F)(F)C4)c(OC(F)(F)F)ccc23)c(Cl)c 1
FCOc1ccc2C(═CC(═O)Nc2c1OCCCCCn3cccn3)Nc4c(Cl)cc(F)cc4Cl
OCCOCCCCCOc1c(OC(F)F)ccc2C(═CC(═O)Nc12)Nc3c(Cl)cc(F)cc3Cl
COc1ccc2C(═CC(═O)Nc2c1OCCCCCCNC(═N)N)Nc3c(Cl)cc(F)cc3Cl
FCOc1ccc2C(═CC(═O)Nc2c1OCCCCCCn3nccn3)Nc4c(Cl)cc(F)cc4Cl
Fc1cc(Cl)c(NC2=CC(═O)Nc3c(OCCCCCCn4ncnn4)c(OC(F)(F)F)ccc23)c(Cl)c1
CN(C)CCOC(═O)COc1c(OCF)ccc2C(═CC(═O)Nc12)Nc3c(Cl)cc(F)cc3Cl
OC(═O)CNC(═O)COc1c(OC(F)(F)F)ccc2C(═CC(═O)Nc12)Nc3c(Cl)cc(F)cc3Cl
OC(═O)[C@H](Cc1ccccc1)NC(═O)COc2c(OC(F)F)ccc3C(═CC(═O)Nc23)Nc4c(Cl)cc (F)cc4Cl
COc1ccc2C(═CC(═O)Nc2c1OCCCC(═O)N3CCNCC3)Nc4c(Cl)cc(F)cc4C1
C(═O)[C@@H]1CCCN1C(═O)CCCOc2c(OC(F)(F)F)ccc3C(═CC(═O)Nc23)Nc4c(C 1)cc(F)cc4Cl
OC[C@H](NC(═O)CCCOc1c(OCF)ccc2C(═CC(═O)Nc12)Nc3c(Cl)cc(F)cc3Cl)C(═O) O
Fc1cc(Cl)c(NC2=CC(═O)Nc3c(OCCCCC(═O)N4CCNCC4)c(OC(F)(F)F)ccc23)c(Cl)c 1
NC(═O)C[C@H](NC(═O)CCCCOc1c(OCF)ccc2C(═CC(═O)Nc12)Nc3c(Cl)cc(F)cc3Cl)C(═O)O
COc1ccc2C(═CC(═O)Nc2c1OCCCCCC(═O)N3CCC[C@H]3C(═O))Nc4c(Cl)cc(F)c c4Cl
OC(═O)[C@H](Cc1ccccc1)NC(═O)CCCCCOc2c(OC(F)(F)F)ccc3C(═CC(═O)Nc23)N c4c(Cl)cc(F)cc4Cl
OC(═O)[C@H](Cc1c[nH]c2ccccc12)NC(═O)CCCCCOc3c(OC(F)F)ccc4C(═CC(═O) Nc34)Nc5c(Cl)cc(F)cc5Cl
CCCN(C)CCCCOc1c(OC)ccc2C(═CC(═O)Nc12)Nc3c(Cl)cc(F)cc3Br
COCCOCCOCCOCCCCOc1c(OC(F)F)ccc2C(═CC(═O)Nc12)Nc3c(Cl)cc(F)cc3Br
CCNCCCCCOc1c(OCF)ccc2C(═CC(═O)Nc12)Nc3c(Cl)cc(F)cc3Br
COc1ccc2C(═CC(═O)Nc2c1OCCCCCN3CCCC(F)C3)Nc4c(Cl)cc(F)cc4Br
CCN(CC)CCCCCCOc1c(OC(F)F)ccc2C(═CC(═O)Nc12)Nc3c(Cl)cc(F)cc3Br
FC1CCN(CCCCCCOc2c(OC(F)(F)F)ccc3C(═CC(═O)Nc23)Nc4c(Cl)cc(F)cc4Br)CC1
COc1ccc2C(═CC(═O)Nc2c1OCCCCCCOc3ccncc3)Nc4c(Cl)cc(F)cc4Br
FCOc1ccc2C(═CC(═O)Nc2c1OCC(═O)N3CCNCC3)Nc4c(Cl)cc(F)cc4Br
NCCCC[C@H](NC(═O)COc1c(OC(F)(F)F)ccc2C(═CC(═O)Nc12)Nc3c(Cl)cc(F)cc3B r)C(═O)O
CN(C)CCOC(═O)CCCOc1c(OCF)ccc2C(═CC(═O)Nc12)Nc3c(Cl)cc(F)cc3Br
CSCC[C@H](NC(═O)CCCOc1c(OC(F)(F)F)ccc2C(═CC(═O)Nc12)Nc3c(Cl)cc(F)cc3Br)C(═O)O
C[N+](C)(C)CCNC(═O)CCCCOc1c(OC(F)(F)F)ccc2C(═CC(═O)Nc12)Nc3c(Cl)cc(F) cc3Br
COc1ccc2C(═CC(═O)Nc2c1OCCCCC(═O)N(C)CCCN(C)C)Nc3c(Cl)cc(F)cc3Br
CN(C)CCCN(C)C(═O)CCCCOc1c(OCF)ccc2C(═CC(═O)Nc12)Nc3c(Cl)cc(F)cc3Br
NC(═O)CCCCCOc1c(OC(F)F)ccc2C(═CC(═O)Nc12)Nc3c(Cl)cc(F)cc3Br
NC(═N)NCCC[C@H](NC(═O)CCCCCOc1c(OC(F)(F)F)ccc2C(═CC(═O)Nc12)Nc3c(Cl)cc(F)cc3Br)C(═O)O
Oc1cc(Cl)c(NC2=CC(═O)Nc3c(OCCCCn4nccn4)c(OCF)ccc23)c(Cl)c1
OCCOCCOCCCCOc1c(OC(F)F)ccc2C(═CC(═O)Nc12)Nc3c(Cl)cc(O)cc3Cl
NCCCCCOc1c(OCF)ccc2C(═CC(═O)Nc12)Nc3c(Cl)cc(O)cc3Cl
CCN(CC)CCCCCOc1c(OC(F)(F)F)ccc2C(═CC(═O)Nc12)Nc3c(Cl)cc(O)cc3Cl
CN(C)CCCCCCOc1c(OCF)ccc2C(═CC(═O)Nc12)Nc3c(Cl)cc(O)cc3Cl
CCCNCCCCCCOc1c(OC(F)F)ccc2C(═CC(═O)Nc12)Nc3c(Cl)cc(O)cc3Cl
COc1ccc2C(═CC(═O)Nc2c1OCCCCCCN3CCC(F)CC3)Nc4c(Cl)cc(O)cc4Cl
Oc1cc(Cl)c(NC2=CC(═O)Nc3c(OCCCCCCOc4cccnc4)c(OC(F)(F)F)ccc23)c(Cl)c1
COc1ccc2C(═CC(═O)Nc2c1OCC(═O)OCCN(C)C)Nc3c(C)cc(O)cc3Cl
OC(═O)CNC(═O)COc1c(OC(F)(F)F)ccc2C(═CC(═O)Nc12)Nc3c(Cl)cc(O)cc3Cl
COc1ccc2C(═CC(═O)Nc2c1OCCCC(═O)NCC(═O)O)Nc3c(Cl)cc(O)cc3Cl
CSCC[C@H](NC(═O)CCCOc1c(OC(F)F)ccc2C(═CC(═O)Nc12)Nc3c(Cl)cc(O)cc3Cl) C(═O)O
NCCCC[C@H](NC(═O)CCCOc1c(OC(F)(F)F)ccc2C(═CC(═O)Nc12)Nc3c(Cl)cc(O)c c3Cl)C(═O)O
OC(═O)[C@H](Cc1cnc[nH]1)NC(═O)CCCOc2c(OCF)ccc3C(═CC(═O)Nc23)Nc4c(Cl)cc(O)cc4Cl
NC(═O)CCCCOc1c(OCF)ccc2C(═CC(═O)Nc12)Nc3c(Cl)cc(O)cc3Cl
OC(═O)CNC(═O)CCCCOc1c(OC(F)F)ccc2C(═CC(═O)Nc12)Nc3c(Cl)cc(O)cc3Cl
C[N+](C)(C)CCNC(═O)CCCCCOc1c(OCF)ccc2C(═CC(═O)Nc12)Nc3c(Cl)cc(O)cc3Cl
COc1ccc2C(═CC(═O)Nc2c1OCCCCCC(═O)NCC(═O)O)Nc3c(Cl)cc(O)cc3C1
C(═O)[C@H](Cc1ccccc1)NC(═O)CCCCCOc2c(OC(F)F)ccc3C(═CC(═O)Nc23)Nc4c(Cl)cc(O)cc4Cl
CCN(CC)CCCCOc1c(OCF)ccc2C(═CC(═O)Nc12)Nc3c(Cl)cc(O)cc3Br
COc1ccc2C(═CC(═O)Nc2c1OCCCCN3CCC(F)(F)C3)Nc4c(Cl)cc(O)cc4Br
Oc1cc(Cl)c(NC2=CC(═O)Nc3c(OCCCCOc4cccnc4)c(OC(F)F)ccc23)c(Br)c1
COc1ccc2C(═CC(═O)Nc2c1OCCCCCN)Nc3c(Cl)cc(O)cc3Br
Oc1cc(Cl)c(NC2=CC(═O)Nc3c(OCCCCCN4CCCC(F)C4)c(OC(F)F)ccc23)c(Br)c1
COCCOCCCCCOc1c(OCF)ccc2C(═CC(═O)Nc12)Nc3c(Cl)cc(O)cc3Br
CCCCN(C)CCCCCCOc1c(OC(F)F)ccc2C(═CC(═O)Nc12)Nc3c(Cl)cc(O)cc3Br
Oc1cc(Cl)c(NC2=CC(═O)Nc3c(OCCCCCCOc4ccccn4)c(OCF)ccc23)c(Br)c1
Oc1cc(Cl)c(NC2=CC(═O)Nc3c(OCC(═O)N4CCNCC4)c(OC(F)(F)F)ccc23)c(Br)c1
COc1ccc2C(═CC(═O)Nc2c1OCC(═O)N[C@@H](CCSC)C(═O)O)Nc3c(Cl)cc(O)cc3Br
NC(═O)CC[C@H](NC(═O)COc1c(OC(F)F)ccc2C(═CC(═O)Nc12)Nc3c(Cl)cc(O)cc3Br)C(═O)O
CN1CCN(CC1)C(═O)CCCOc2c(OC(F)(F)F)ccc3C(═CC(═O)Nc23)Nc4c(Cl)cc(O)cc4Br
COc1ccc2C(═CC(═O)Nc2c1OCCCC(═O)N[C@@H](C)C(═O)O)Nc3c(Cl)cc(O)cc3Br
CN(C)CCCN(C)C(═O)CCCCOc1c(OC(F)F)ccc2C(═CC(═O)Nc12)Nc3c(Cl)cc(O)cc3Br
OC(═O)CNC(═O)CCCCOc1c(OCF)ccc2C(═CC(═O)Nc12)Nc3c(Cl)cc(O)cc3Br
COc1ccc2C(═CC(═O)Nc2c1OCCCCC(═O)N[C@@H](CCC(═O)O)C(═O))Nc3c(Cl) cc(O)cc3Br
CN(C)CCN(C)C(═O)CCCCCOc1c(OCF)ccc2C(═CC(═O)Nc12)Nc3c(Cl)cc(O)cc3Br
CN(CCC[N+](C)(C)C)C(═O)CCCCCOc1c(OC(F)(F)F)ccc2C(═CC(═O)Nc12)Nc3c(Cl)cc(O)cc3Br
OC(═O)[C@H](Cc1ccccc1)NC(═O)CCCCCOc2c(OC(F)F)ccc3C(═CC(═O)Nc23)Nc4c(Cl)cc(O)cc4Br
COc1ccc2C(═CC(═O)Nc2c1OCCCCN(C)C)Nc3c(Cl)cc(cc3Cl)C(═O)O
OC(═O)c1cc(Cl)c(NC2=CC(═O)Nc3c(OCCCCOc4cccnc4)c(OC(F)(F)F)ccc23)c(Cl)c1
COCCOCCOCCOCCCCOc1c(OC(F)F)ccc2C(═CC(═O)Nc12)Nc3c(Cl)cc(cc3Cl)C(═O)O
NC(═N)NCCCCCOc1c(OCF)ccc2C(═CC(═O)Nc12)Nc3c(Cl)cc(cc3Cl)C(═O)O
COc1ccc2C(═CC(═O)Nc2c1OCCCCCN3CCCC(F)C3)Nc4c(Cl)cc(cc4Cl)C(═O)O
OC(═O)c1cc(Cl)c(NC2=CC(═O)Nc3c(OCCCCCN4CCC(F)(F)C4)c(OC(F)F)ccc23)c(Cl)c1
CNCCCCCCOc1c(OCF)ccc2C(═CC(═O)Nc12)Nc3c(Cl)cc(cc3Cl)C(═O)O
CCN(C)CCCCCCOc1c(OC(F)(F)F)ccc2C(═CC(═O)Nc12)Nc3c(Cl)cc(cc3Cl)C(═O)O
COCCOCCCCCCOc1c(OC)ccc2C(═CC(═O)Nc12)Nc3c(Cl)cc(cc3Cl)C(═O)O
COc1ccc2C(═CC(═O)Nc2c1OCC(═O)N3CCC[C@H]3C(═O))Nc4c(Cl)cc(cc4Cl)C(═O)O
COc1ccc2C(═CC(═O)Nc2c1OCCCC(═O)NCC(═O)O)Nc3c(Cl)cc(cc3Cl)C(═O)O
OC(═O)[C@H](Cc1ccccc1)NC(═O)CCCOc2c(OCF)ccc3C(═CC(═O)Nc23)Nc4c(Cl)c c(cc4Cl)C(═O)O
CN(C)CCCN(C)C(═O)CCCCOc1c(OCF)ccc2C(═CC(═O)Nc12)Nc3c(Cl)cc(cc3Cl)C(═O)O
COc1ccc2C(═CC(═O)Nc2c1OCCCCC(═O)N[C@@H](CC(C)C)C(═O)O)Nc3c(Cl)cc(cc3Cl)C(═O)O
C[C@H](NC(═O)CCCCCOc1c(OCF)ccc2C(═CC(═O)Nc12)Nc3c(Cl)cc(cc3Cl)C(═O) O)C(═O)O
COc1ccc2C(═CC(═O)Nc2c1OCCCCNC(═N)N)Nc3c(Cl)cc(cc3Br)C(═O)O
OC(═O)c1cc(Cl)c(NC2=CC(═O)Nc3c(OCCCCN4CCC(F)(F)C4)c(OC(F)F)ccc23)c(Br)c1
CN(C)CCCCCOc1c(OC(F)(F)F)ccc2C(═CC(═O)Nc12)Nc3c(Cl)cc(cc3Br)C(═O)O
COc1ccc2C(═CC(═O)Nc2c1OCCCCCN3CCOCC3)Nc4c(Cl)cc(cc4Br)C(═O)O
OC(═O)c1cc(Cl)c(NC2=CC(═O)Nc3c(OCCCCCCn4ncnn4)c(OCF)ccc23)c(Br)c1
COCCOCCCCCCOc1c(OC(F)F)ccc2C(═CC(═O)Nc12)Nc3c(Cl)cc(cc3Br)C(═O)O
NC(═O)COc1c(OC(F)F)ccc2C(═CC(═O)Nc12)Nc3c(C)cc(cc3Br)C(═O)O
CN(CCC[N+](C)(C)C)C(═O)COc1c(OCF)ccc2C(═CC(═O)Nc12)Nc3c(Cl)cc(cc3Br)C (═O)O
CN1CCN(CC1)C(═O)COc2c(OC(F)(F)F)ccc3C(═CC(═O)Nc23)Nc4c(Cl)cc(cc4Br)C(═O)O
C[N+](C)(C)CCNC(═O)CCCOc1c(OC(F)F)ccc2C(═CC(═O)Nc12)Nc3c(Cl)cc(cc3Br) C(═O)O
CN(C)CCCN(C)C(═O)CCCCOc1c(OC(F)F)ccc2C(═CC(═O)Nc12)Nc3c(Cl)cc(cc3Br) C(═O)O
OC(═O)[C@@H]1CCCN1C(═O)CCCCCOc2c(OCF)ccc3C(═CC(═O)Nc23)Nc4c(Cl)c c(cc4Br)C(═O)O
COc1ccc2C(═CC(═O)Nc2c1OCCCCCC(═O)N[C@@H](CCCNC(═N)N)C(═O)O)Nc3c(Cl)cc(cc3Br)C(═O)O
CCCN(C)CCCCOc1c(OC)ccc2C(═CC(═O)Nc12)Nc3c(Br)cccc3Br
FC(F)(F)Oc1ccc2C(═CC(═O)Nc2c1OCCCCn3cncn3)Nc4c(Br)cccc4Br
COCCOCCOCCOCCCCOc1c(OC(F)F)ccc2C(═CC(═O)Nc12)Nc3c(Br)cccc3Br
FCOc1ccc2C(═CC(═O)Nc2c1OCCCCCN3CCOCC3)Nc4c(Br)cccc4Br
COCCOCCCCCOc1c(OC)ccc2C(═CC(═O)Nc12)Nc3c(Br)cccc3Br
CCN(CC)CCCCCCOc1c(OC(F)F)ccc2C(═CC(═O)Nc12)Nc3c(Br)cccc3Br
FC(F)(F)Oc1ccc2C(═CC(═O)Nc2c1OCCCCCCN3CCOCC3)Nc4c(Br)cccc4Br
FCOc1ccc2C(═CC(═O)Nc2c1OCCCCCCn3cccn3)Nc4c(Br)cccc4Br
CN1CCN(CC1)C(═O)COc2c(OC(F)F)ccc3C(═CC(═O)Nc23)Nc4c(Br)cccc4Br
C[C@H](NC(═O)COc1c(OCF)ccc2C(═CC(═O)Nc12)Nc3c(Br)cccc3Br)C(═O)O
C[N+](C)(C)CCNC(═O)CCCOc1c(OCF)ccc2C(═CC(═O)Nc12)Nc3c(Br)cccc3Br
C[C@H](NC(═O)CCCOc1c(OC(F)F)ccc2C(═CC(═O)Nc12)Nc3c(Br)cccc3Br)C(═O) O
C(═O)[C@H](Cc1c[nH]c2ccccc12)NC(═O)CCCOc3c(OC(F)(F)F)ccc4C(═CC(═O)N c34)Nc5c(Br)cccc5Br
C[N+](C)(C)CCNC(═O)CCCCOc1c(OCF)ccc2C(═CC(═O)Nc12)Nc3c(Br)cccc3Br
COc1ccc2C(═CC(═O)Nc2c1OCCCCC(═O)N[C@@H](CC(C)C)C(═O)O)Nc3c(Br)ccc c3Br
CN(C)CCN(C)C(═O)CCCCCOc1c(OC(F)(F)F)ccc2C(═CC(═O)Nc12)Nc3c(Br)cccc3Br
CN(CC[N+](C)(C)C)C(═O)CCCCCOc1c(OC(F)F)ccc2C(═CC(═O)Nc12)Nc3c(Br)ccc c3Br
FCOc1ccc2C(═CC(═O)Nc2c1OCCCCCC(═O)N3CCNCC3)Nc4c(Br)cccc4Br
COc1ccc2C(═CC(═O)Nc2c1OCCCCCC(═O)N[C@@H](CC(═O)N)C(═O)O)Nc3c(Br) cccc3Br
FCOc1ccc2C(═CC(═O)Nc2c1OCCCCN3CCCC(F)(F)C3)Nc4c(Br)cc(F)cc4Br
FC(F)Oc1ccc2C(═CC(═O)Nc2c1OCCCCN3CCC(F)(F)CC3)Nc4c(Br)cc(F)cc4Br
COCCN(C)CCCCCOc1c(OC(F)F)ccc2C(═CC(═O)Nc12)Nc3c(Br)cc(F)cc3Br
COc1ccc2C(═CC(═O)Nc2c1OCCCCCN3CCOCC3)Nc4c(Br)cc(F)cc4Br
Fc1cc(Br)c(NC2=CC(═O)Nc3c(OCCCCCn4cccc4)c(OC(F)(F)F)ccc23)c(Br)c1
FCOc1ccc2C(═CC(═O)Nc2c1OCCCCCn3cnnn3)Nc4c(Br)cc(F)cc4Br
CNCCCCCCOc1c(OCF)ccc2C(═CC(═O)Nc12)Nc3c(Br)cc(F)cc3Br
FC(F)Oc1ccc2C(═CC(═O)Nc2c1OCCCCCCn3ccnn3)Nc4c(Br)cc(F)cc4Br
NCCCC[C@H](NC(═O)COc1c(OCF)ccc2C(═CC(═O)Nc12)Nc3c(Br)cc(F)cc3Br)C(═O)O
C(═O)[C@H](Cc1cnc[nH]1)NC(═O)COc2c(OC(F)(F)F)ccc3C(═CC(═O)Nc23)Nc4c (Br)cc(F)cc4Br
COc1ccc2C(═CC(═O)Nc2c1OCCCC(═O)NCCN(C)C)Nc3c(Br)cc(F)cc3Br
CN(CC[N+](C)(C)C)C(═O)CCCOc1c(OC(F)F)ccc2C(═CC(═O)Nc12)Nc3c(Br)cc(F)c c3Br
OC(═O)[C@H](Cc1ccccc1)NC(═O)CCCOc2c(OC(F)(F)F)ccc3C(═CC(═O)Nc23)Nc4c(Br)cc(F)cc4Br
NC(═O)CCCCOc1c(OC(F)F)ccc2C(═CC(═O)Nc12)Nc3c(Br)cc(F)cc3Br
CN(C)CCNC(═O)CCCCOc1c(OC(F)(F)F)ccc2C(═CC(═O)Nc12)Nc3c(Br)cc(F)cc3Br
COc1ccc2C(═CC(═O)Nc2c1OCCCCC(═O)N3CCN(C)CC3)Nc4c(Br)cc(F)cc4Br
OC(═O)CNC(═O)CCCCOc1c(OCF)ccc2C(═CC(═O)Nc12)Nc3c(Br)cc(F)cc3Br
COc1ccc2C(═CC(═O)Nc2c1OCCCCCC(═O)N3CCNCC3)Nc4c(Br)cc(F)cc4Br
C[C@@H](O)[C@H](NC(═O)CCCCCOc1c(OC(F)F)ccc2C(═CC(═O)Nc12)Nc3c(Br) cc(F)cc3Br)C(═O)O
OC(═O)[C@H](Cc1cnc[nH]1)NC(═O)CCCCCOc2c(OCF)ccc3C(═CC(═O)Nc23)Nc4c (Br)cc(F)cc4Br
CCNCCCCOc1c(OC(F)F)ccc2C(═CC(═O)Nc12)Nc3c(Br)cc(O)cc3Br
Oc1cc(Br)c(NC2=CC(═O)Nc3c(OCCCCN4CCCC4)c(OC(F)(F)F)ccc23)c(Br)c1
COc1ccc2C(═CC(═O)Nc2c1OCCCCN3CCC(F)CC3)Nc4c(Br)cc(O)cc4Br
CCCNCCCCCOc1c(OC(F)F)ccc2C(═CC(═O)Nc12)Nc3c(Br)cc(O)cc3Br
Oc1cc(Br)c(NC2=CC(═O)Nc3c(OCCCCCN4CCCC4)c(OC(F)(F)F)ccc23)c(Br)c1
COc1ccc2C(═CC(═O)Nc2c1OCCCCCN3CCC(F)(F)C3)Nc4c(Br)cc(O)cc4Br
Oc1cc(Br)c(NC2=CC(═O)Nc3c(OCCCCCOc4ccccn4)c(OCF)ccc23)c(Br)c1
CN(C)CCCCCCOc1c(OC(F)F)ccc2C(═CC(═O)Nc12)Nc3c(Br)cc(O)cc3Br
Oc1cc(Br)c(NC2=CC(═O)Nc3c(OCCCCCCN4CCOCC4)c(OCF)ccc23)c(Br)c1
Oc1cc(Br)c(NC2=CC(═O)Nc3c(OCCCCCCn4cccc4)c(OC(F)(F)F)ccc23)c(Br)c1
COc1ccc2C(═CC(═O)Nc2c1OCC(═O)N(C)CCCN(C)C)Nc3c(Br)cc(O)cc3Br
CN1CCN(CC1)C(═O)COc2c(OC(F)(F)F)ccc3C(═CC(═O)Nc23)Nc4c(Br)cc(O)cc4Br
NCCCC[C@H](NC(═O)COc1c(OC(F)F)ccc2C(═CC(═O)Nc12)Nc3c(Br)cc(O)cc3Br) C(═O)O
OC(═O)[C@H](Cc1cnc[nH]1)NC(═O)COc2c(OCF)ccc3C(═CC(═O)Nc23)Nc4c(Br)cc (O)cc4Br
OC(═O)CNC(═O)CCCOc1c(OC(F)(F)F)ccc2C(═CC(═O)Nc12)Nc3c(Br)cc(O)cc3Br
CSCC[C@H](NC(═O)CCCOc1c(OCF)ccc2C(═CC(═O)Nc12)Nc3c(Br)cc(O)cc3Br)C(═O)O
NC(═N)NCCC[C@H](NC(═O)CCCOc1c(OC(F)F)ccc2C(═CC(═O)Nc12)Nc3c(Br)cc(O)cc3Br)C(═O)O
COc1ccc2C(═CC(═O)Nc2c1OCCCCC(═O)N[C@@H](CC(═O)N)C(═O))Nc3c(Br)cc(O)cc3Br
CN(C)CCNC(═O)CCCCOc1c(OC(F)F)ccc2C(═CC(═O)Nc12)Nc3c(Br)cc(O)cc3Br
C[C@@H](O)[C@H](NC(═O)CCCCCOc1c(OCF)ccc2C(═CC(═O)Nc12)Nc3c(Br)cc(O)cc3Br)C(═O)O
OC(═O)[C@H](Cc1cnc[nH]1)NC(═O)CCCCCOc2c(OC(F)(F)F)ccc3C(═CC(═O)Nc23)Nc4c(Br)cc(O)cc4Br
CNCCCCOc1c(OCF)ccc2C(═CC(═O)Nc12)Nc3c(Br)cc(cc3Br)C(═O)O
CCN(C)CCCCOc1c(OC)ccc2C(═CC(═O)Nc12)Nc3c(Br)cc(cc3Br)C(═O)O
OC(═O)c1cc(Br)c(NC2=CC(═O)Nc3c(OCCCCN4CCC(F)C4)c(OC(F)(F)F)ccc23)c(Br)c1
C(═O)c1cc(Br)c(NC2=CC(═O)Nc3c(OCCCCCN4CCCC(F)(F)C4)c(OCF)ccc23)c(B r)c1
C(═O)c1cc(Br)c(NC2=CC(═O)Nc3c(OCCCCCn4cnnn4)c(OC(F)F)ccc23)c(Br)c1
COCCOCCOCCCCCOc1c(OC(F)(F)F)ccc2C(═CC(═O)Nc12)Nc3c(Br)cc(cc3Br)C(═O)O
CNCCCCCCOc1c(OC(F)(F)F)ccc2C(═CC(═O)Nc12)Nc3c(Br)cc(cc3Br)C(═O)O
COCCN(C)CCCCCCOc1c(OC(F)F)ccc2C(═CC(═O)Nc12)Nc3c(Br)cc(cc3Br)C(═O)O
COc1ccc2C(═CC(═O)Nc2c1OCCCCCCN3CCCC(F)(F)C3)Nc4c(Br)cc(cc4Br)C(═O) O
OCCOCCCCCCOc1c(OCF)ccc2C(═CC(═O)Nc12)Nc3c(Br)cc(cc3Br)C(═O)O
CN(C)CCNC(═O)CCCOc1c(OCF)ccc2C(═CC(═O)Nc12)Nc3c(Br)cc(cc3Br)C(═O)O
OC(═O)c1cc(Br)c(NC2=CC(═O)Nc3c(OCCCC(═O)N4CCNCC4)c(OC(F)F)ccc23)c(B r)c1
COc1ccc2C(═CC(═O)Nc2c1OCCCC(═O)N[C@@H](CO)C(═O))Nc3c(Br)cc(cc3Br) C(═O)O
CN(C)CCNC(═O)CCCCOc1c(OCF)ccc2C(═CC(═O)Nc12)Nc3c(Br)cc(cc3Br)C(═O)O
CN(CC[N+](C)(C)C)C(═O)CCCCCOc1c(OC(F)(F)F)ccc2C(═CC(═O)Nc12)Nc3c(Br) cc(cc3Br)C(═O)O
C[C@H](NC(═O)CCCCCOc1c(OCF)ccc2C(═CC(═O)Nc12)Nc3c(Br)cc(cc3Br)C(═O) O)C(═O)O
COc1ccc2C(═CC(═O)Nc2c1CCCCCC(═O)N[C@@H](CCCCN)C(═O)O)Nc3c(Br)c c(cc3Br)C(═O)O
CCN(CC)CCCCOc1c(OC(F)F)ccc2C(═CC(═O)Nc12)Nc3c(C)cncc3C
CCCCN(C)CCCCOc1c(OCF)ccc2C(═CC(═O)Nc12)Nc3c(C)cncc3C
COc1ccc2C(═CC(═O)Nc2c1OCCCCN3CCCC(F)(F)C3)Nc4c(C)cncc4C
Cc1cncc(C)c1NC2=CC(═O)Nc3c(OCCCCn4nccn4)c(OC(F)F)ccc23
Cc1cncc(C)c1NC2=CC(═O)Nc3c(OCCCCn4cncn4)c(OC(F)(F)F)ccc23
CNCCCCCOc1c(OC(F)F)ccc2C(═CC(═O)Nc12)Nc3c(C)cncc3C
COCCN(C)CCCCCOc1c(OCF)ccc2C(═CC(═O)Nc12)Nc3c(C)cncc3C
COc1ccc2C(═CC(═O)Nc2c1OCCCCCN3CCC(F)CC3)Nc4c(C)cncc4C
Cc1cncc(C)c1NC2=CC(═O)Nc3c(OCCCCCn4nccn4)c(OC(F)(F)F)ccc23
Cc1cncc(C)c1NC2=CC(═O)Nc3c(OCCCCCOc4cccnc4)c(OC(F)F)ccc23
CCNCCCCCCOc1c(OCF)ccc2C(═CC(═O)Nc12)Nc3c(C)cncc3C
CCN(C)CCCCCCOc1c(OC(F)F)ccc2C(═CC(═O)Nc12)Nc3c(C)cncc3C
CCCCN(C)CCCCCCOc1c(OC(F)(F)F)ccc2C(═CC(═O)Nc12)Nc3c(C)cncc3C
C[C@H](NC(═O)COc1c(OC(F)F)ccc2C(═CC(═O)Nc12)Nc3c(C)cncc3C)C(═O)O
Cc1cncc(C)c1NC2=CC(═O)Nc3c(OCC(═O)N4CCC[C@H]4C(═O)O)c(OC(F)(F)F)cc c23
COc1ccc2C(═CC(═O)Nc2c1OCC(═O)N[C@@H](Cc3ccccc3)C(═O)O)Nc4c(C)cncc4C
C[C@@H](O)[C@H](NC(═O)COc1c(OCF)ccc2C(═CC(═O)Nc12)Nc3c(C)cncc3C)C(═O)O
Cc1cncc(C)c1NC2=CC(═O)Nc3c(OCCCC(═O)OCC[N+](C)(C)C)c(OC(F)F)ccc23
CN(CC[N+](C)(C)C)C(═O)CCCOc1c(OC(F)(F)F)ccc2C(═CC(═O)Nc12)Nc3c(C)cncc 3C
CN(C)CCCN(C)C(═O)CCCOc1c(OCF)ccc2C(═CC(═O)Nc12)Nc3c(C)cncc3C
COc1ccc2C(═CC(═O)Nc2c1OCCCC(═O)N3CCN(C)CC3)Nc4c(C)cncc4C
Cc1cncc(C)c1NC2=CC(═O)Nc3c(OCCCCC(═O)N)c(OC(F)F)ccc23
COc1ccc2C(═CC(═O)Nc2c1OCCCCC(═O)N3CCNCC3)Nc4c(C)cncc4C
Cc1cncc(C)c1NC2=CC(═O)Nc3c(OCCCCC(═O)N[C@@H](CO)C(═O)O)c(OCF)ccc 23
C[C@@H](O)[C@H](NC(═O)CCCCOc1c(OC(F)(F)F)ccc2C(═CC(═O)Nc12)Nc3c(C) cncc3C)C(═O)O
Cc1cncc(C)c1NC2=CC(═O)Nc3c(OCCCCCC(═O)N4CCNCC4)c(OCF)ccc23
COc1ccc2C(═CC(═O)Nc2c1OCCCCCC(═O)NCC(═O)O)Nc3c(C)cncc3C
Cc1cncc(C)c1NC2=CC(═O)Nc3c(OCCCCCC(═O)N4CCC[C@H]4C(═O)O)c(OC(F)F)ccc23
CSCC[C@H](NC(═O)CCCCCOc1c(OC(F)(F)F)ccc2C(═CC(═O)Nc12)Nc3c(C)cncc3C)C(═O)O
Cc1cncc(F)c1NC2=CC(═O)Nc3c(OCCCCN4CCCCC4)c(OC(F)F)ccc23
COc1ccc2C(═CC(═O)Nc2c1OCCCCN3CCC(F)(F)CC3)Nc4c(C)cncc4F
COCCOCCOCCOCCCCOc1c2NC(═O)C═C(Nc3c(C)cncc3F)c2ccc1OCF
COCCOCCOCCOCCCCOc1c(OC(F)(F)F)ccc2C(═CC(═O)Nc12)Nc3c(C)cncc3F
CN(C)CCCCCOc1c(OC(F)F)ccc2C(═CC(═O)Nc12)Nc3c(C)cncc3F
Cc1cncc(F)c1NC2=CC(═O)Nc3c(OCCCCCN4CCC(F)(F)CC4)c(OCF)ccc23
Cc1cncc(F)c1NC2=CC(═O)Nc3c(OCCCCCn4cccn4)c(OC(F)(F)F)ccc23
COc1ccc2C(═CC(═O)Nc2c1OCCCCCOCCO)Nc3c(C)cncc3F
CNCCCCCCOc1c(OC(F)F)ccc2C(═CC(═O)Nc12)Nc3c(C)cncc3F
CCN(CC)CCCCCCOc1c(OCF)ccc2C(═CC(═O)Nc12)Nc3c(C)cncc3F
Cc1cncc(F)c1NC2=CC(═O)Nc3c(OCCCCCCN4CCCCC4)c(OC(F)(F)F)ccc23
COCCOCCOCCCCCCOc1c(OC)ccc2C(═CC(═O)Nc12)Nc3c(C)cncc3F
Cc1cncc(F)c1NC2=CC(═O)Nc3c(OCC(═O)OCC[N+](C)(C)C)c(OCF)ccc23
CN1CCN(CC1)C(═O)COc2c(OC(F)F)ccc3C(═CC(═O)Nc23)Nc4c(C)cncc4F
COc1ccc2C(═CC(═O)Nc2c1OCC(═O)N[C@@H](C(C)C)C(═O)O)Nc3c(C)cncc3F
Cc1cncc(F)c1NC2=CC(═O)Nc3c(OCC(═O)N[C@@H](Cc4c[nH]c5ccccc45)C(═O)O) c(OC(F)(F)F)ccc23
Cc1cncc(F)c1NC2=CC(═O)Nc3c(OCCCC(═O)NCC[N+](C)(C)C)c(OC(F)F)ccc23
CN1CCN(CC1)C(═O)CCCOc2c(OCF)ccc3C(═CC(═O)Nc23)Nc4c(C)cncc4F
CN1CCN(CC1)C(═O)CCCOc2c(OC(F)(F)F)ccc3C(═CC(═O)Nc23)Nc4c(C)cncc4F
COc1ccc2C(═CC(═O)Nc2c1OCCCCC(═O)N[C@@H](C)C(═O)O)Nc3c(C)cncc3F
CN(C)CCN(C)C(═O)CCCCOc1c(OCF)ccc2C(═CC(═O)Nc12)Nc3c(C)cncc3F
CN(CC[N+](C)(C)C)C(═O)CCCCOc1c(OC(F)(F)F)ccc2C(═CC(═O)Nc12)Nc3c(C)cn cc3F
CN1CCN(CC1)C(═O)CCCCOc2c(OC(F)(F)F)ccc3C(═CC(═O)Nc23)Nc4c(C)cncc4F
CSCC[C@H](NC(═O)CCCCOc1c(OC(F)F)ccc2C(═CC(═O)Nc12)Nc3c(C)cncc3F)C(═O)O
COc1ccc2C(═CC(═O)Nc2c1OCCCCC(═O)N[C@@H](CCC(═O)N)C(═O)O)Nc3c(C) cncc3F
CN(C)CCN(C)C(═O)CCCCCOc1c(OC(F)F)ccc2C(═CC(═O)Nc12)Nc3c(C)cncc3F
Cc1cncc(F)c1NC2=CC(═O)Nc3c(OCCCCCC(═O)N[C@@H](CO)C(═O)O)c(OCF)cc c23
Cc1cncc(F)c1NC2=CC(═O)Nc3c(OCCCCCC(═O)N[C@@H](CO)C(═O)O)c(OC(F)(F)F)ccc23
COc1ccc2C(═CC(═O)Nc2cOCCCCCC(═O)N[C@@H](CCCCN)C(═O)O)Nc3c(C)cn cc3F
Cc1cncc(Cl)c1NC2=CC(═O)Nc3c(OCCCCN4CCOCC4)c(OC(F)(F)F)ccc23
Cc1cncc(Cl)c1NC2=CC(═O)Nc3c(OCCCCN4CCC(F)CC4)c(OC(F)F)ccc23
COc1ccc2C(═CC(═O)Nc2c1OCCCCN3CCC(F)(F)C3)Nc4c(C)cncc4Cl
Cc1cncc(Cl)c1NC2=CC(═O)Nc3c(OCCCCOc4cccnc4)c(OCF)ccc23
CCN(CC)CCCCCOc1c(OC(F)(F)F)ccc2C(═CC(═O)Nc12)Nc3c(C)cncc3Cl
Cc1cncc(Cl)c1NC2=CC(═O)Nc3c(OCCCCCn4ccnc4)c(OC(F)F)ccc23
COc1ccc2C(═CC(═O)Nc2c1OCCCCCn3ccnn3)Nc4c(C)cncc4Cl
Cc1cncc(Cl)c1NC2=CC(═O)Nc3c(OCCCCCOc4ccncc4)c(OCF)ccc23
CNCCCCCCOc1c(OC)ccc2C(═CC(═O)Nc12)Nc3c(C)cncc3Cl
Cc1cncc(Cl)c1NC2=CC(═O)Nc3c(OCCCCCCN4CCCC(F)(F)C4)c(OC(F)(F)F)ccc23
Cc1cncc(Cl)c1NC2=CC(═O)Nc3c(OCCCCCCOc4ccncc4)c(OC(F)F)ccc23
Cc1cncc(Cl)c1NC2=CC(═O)Nc3c(OCCCCCCOCCO)c(OCF)ccc23
COc1ccc2C(═CC(═O)Nc2c1OCC(═O)OCCN(C)C)Nc3c(C)cncc3Cl
Cc1cncc(Cl)c1NC2=CC(═O)Nc3c(OCC(═O)OCC[N+](C)(C)C)c(OC(F)F)ccc23
Cc1cncc(Cl)c1NC2=CC(═O)Nc3c(OCC(═O)OCC[N+](C)(C)C)c(OC(F)(F)F)ccc23
C[C@@H](O)[C@H](NC(═O)COc1c(OCF)ccc2C(═CC(═O)Nc12)Nc3c(C)cncc3Cl)C (═O)O
CN(C)CCCN(C)C(═O)CCCOc1c(OC(F)(F)F)ccc2C(═CC(═O)Nc12)Nc3c(C)cncc3Cl
CN(CCC[N+](C)(C)C)C(═O)CCCOc1c(OC(F)F)ccc2C(═CC(═O)Nc12)Nc3c(C)cncc3Cl
COc1ccc2C(═CC(═O)Nc2c1OCCCC(═O)N[C@@H](C(C)C)C(═O)O)Nc3c(C)cncc3C 1
Cc1cncc(Cl)c1NC2=CC(═O)Nc3c(OCCCC(═O)N[C@@H](CCC(═O)N)C(═O)O)c(O CF)ccc23
CN(C)CCNC(═O)CCCCOc1c(OC(F)(F)F)ccc2C(═CC(═O)Nc12)Nc3c(C)cncc3Cl
COc1ccc2C(═CC(═O)Nc2c1OCCCCC(═O)NCC[N+](C)(C)C)Nc3c(C)cncc3Cl
Cc1cncc(Cl)c1NC2=CC(═O)Nc3c(OCCCCC(═O)N[C@@H](CCCNC(═N)N)C(═O)O)c(OCF)ccc23
Cc1cncc(Cl)c1NC2=CC(═O)Nc3c(OCCCCC(═O)N[C@@H](CC(═O)O)C(═O)O)c(O C(F)F)ccc23
CN(C)CCOC(═O)CCCCCOc1c(OC(F)F)ccc2C(═CC(═O)Nc12)Nc3c(C)cncc3Cl
Cc1cncc(Cl)c1NC2=CC(═O)Nc3c(OCCCCCC(═O)NCC(═O)O)c(OCF)ccc23
C[C@H](NC(═O)CCCCCOc1c(OC(F)(F)F)ccc2C(═CC(═O)Nc12)Nc3c(C)cncc3Cl)C(═O)O
Cc1cncc(Cl)c1NC2=CC(═O)Nc3c(OCCCCCC(═O)N[C@@H](Cc4c[nH]c5ccccc45)C (═O)O)c(OCF)ccc23
COc1ccc2C(═CC(═O)Nc2c1OCCCCCC(═O)N[C@@H](Cc3cnc[nH]3)C(═O)O)Nc4c(C)cncc4Cl
Cc1cncc(Cl)c1NC2=CC(═O)Nc3c(OCCCCCC(═O)N[C@@H](Cc4cnc[nH]4)C(═O)O)c(OC(F)F)ccc23
Cc1cncc(Br)c1NC2=CC(═O)Nc3c(OCCCCN4CCCC(F)C4)c(OC(F)(F)F)ccc23
Cc1cncc(Br)c1NC2=CC(═O)Nc3c(OCCCCN4CCCC(F)(F)C4)c(OC(F)F)ccc23
Cc1cncc(Br)c1NC2=CC(═O)Nc3c(OCCCCN4CCC(F)(F)C4)c(OCF)ccc23
Cc1cncc(Br)c1NC2=CC(═O)Nc3c(OCCCOc4ccncc4)c(OCF)ccc23
COc1ccc2C(═CC(═O)Nc2c1OCCCCOCCO)Nc3c(C)cncc3Br
CN(C)CCCCCOc1c(OC(F)F)ccc2C(═CC(═O)Nc12)Nc3c(C)cncc3Br
CCN(CC)CCCCCOc1c(OCF)ccc2C(═CC(═O)Nc12)Nc3c(C)cncc3Br
CCCCCN(C)CCCCCOc1c(OC)ccc2C(═CC(═O)Nc12)Nc3c(C)cncc3Br
Cc1cncc(Br)c1NC2=CC(═O)Nc3c(OCCCCCN4CCOCC4)c(OC(F)(F)F)ccc23
COCCOCCOCCOCCCCCOc1c(OC(F)F)ccc2C(═CC(═O)Nc12)Nc3c(C)cncc3Br
Cc1cncc(Br)c1NC2=CC(═O)Nc3c(OCCCCCCN4CCC(F)CC4)c(OCF)ccc23
Cc1cncc(Br)c1NC2=CC(═O)Nc3c(OCCCCCCN4CCCC(F)(F)C4)c(OC(F)F)ccc23
COc1ccc2C(═CC(═O)Nc2c1OCCCCCCn3ccnn3)Nc4c(C)cncc4Br
Cc1cncc(Br)c1NC2=CC(═O)Nc3c(OCCCCCCOc4ccncc4)c(OC(F)F)ccc23
COCCOCCOCCCCCCOc1c(OC(F)(F)F)ccc2C(═CC(═O)Nc12)Nc3c(C)cncc3Br
CN(C)CCOC(═O)COc1c(OCF)ccc2C(═CC(═O)Nc12)Nc3c(C)cncc3Br
COc1ccc2C(═CC(═O)Nc2c1OCC(═O)N(C)CC[N+](C)(C)C)Nc3c(C)cncc3Br
Cc1cncc(Br)c1NC2=CC(═O)Nc3c(OCC(═O)N[C@@H](Cc4c[nH]c5ccccc45)C(═O)O)c(OC(F)(F)F)ccc23
Cc1cncc(Br)c1NC2=CC(═O)Nc3c(OCC(═O)N[C@@H](CC(═O)O)C(═O)O)c(OC(F) F)ccc23
CC[C@H](C)[C@H](NC(═O)CCCOc1c(OC(F)F)ccc2C(═CC(═O)Nc12)Nc3c(C)cncc 3Br)C(═O)O
CC[C@H](C)[C@H](NC(═O)CCCOc1c(OC(F)(F)F)ccc2C(═CC(═O)Nc12)Nc3c(C)cn cc3Br)C(═O)O
COc1ccc2C(═CC(═O)Nc2c1OCCCC(═O)N3CCC[C@H]3C(═O)O)Nc4c(C)cncc4Br
Cc1cncc(Br)c1NC2=CC(═O)Nc3c(OCCCC(═O)N[C@@H](CCCNC(═N)N)C(═O)O)c (OCF)ccc23
CC(C)[C@H](NC(═O)CCCCOc1c(OCF)ccc2C(═CC(═O)Nc12)Nc3c(C)cncc3Br)C(═O)O
C[C@@H](O)[C@H](NC(═O)CCCCOc1c(OC(F)F)ccc2C(═CC(═O)Nc12)Nc3c(C)cn cc3Br)C(═O)O
C[C@@H](O)[C@H](NC(═O)CCCCOc1c(OC(F)(F)F)ccc2C(═CC(═O)Nc12)Nc3c(C) cncc3Br)C(═O)O
COc1ccc2C(═CC(═O)Nc2c1OCCCCCC(═O)N[C@@H](Cc3cnc[nH]3)C(═O)O)Nc4c(C)cncc4Br
CN(C)CCNC(═O)CCCCCOc1c(OC(F)(F)F)ccc2C(═CC(═O)Nc12)Nc3c(C)cncc3Br
CN(CCC[N+](C)(C)C)C(═O)CCCCCOc1c(OC(F)F)ccc2C(═CC(═O)Nc12)Nc3c(C)cn cc3Br
Cc1cncc(Br)c1NC2=CC(═O)Nc3c(OCCCCCC(═O)N4CCNCC4)c(OCF)ccc23
COc1ccc2C(═CC(═O)Nc2c1OCCCCCC(═O)N3CCC[C@H]3C(═O))Nc4c(C)cncc4Br
FCOc1ccc2C(═CC(═O)Nc2c1OCCCCN3CCCCC3)Nc4c(F)cncc4F
FC(F)Oc1ccc2C(═CC(═O)Nc2c1OCCCCN3CCCC(F)C3)Nc4c(F)cncc4F
FC1CCN(CCCCOc2c(OC(F)(F)F)ccc3C(═CC(═O)Nc23)Nc4c(F)cncc4F)C1
COc1ccc2C(═CC(═O)Nc2c1OCCCCn3nccn3)Nc4c(F)cncc4F
CCCNCCCCCOc1c(OC(F)(F)F)ccc2C(═CC(═O)Nc12)Nc3c(F)cncc3F
CCN(C)CCCCCOc1c(OC(F)F)ccc2C(═CC(═O)Nc12)Nc3c(F)cncc3F
COCCN(C)CCCCCOc1c(OC)ccc2C(═CC(═O)Nc12)Nc3c(F)cncc3F
FCOc1ccc2C(═CC(═O)Nc2c1OCCCCCn3cnnc3)Nc4c(F)cncc4F
COCCN(C)CCCCCCOc1c(OC)ccc2C(═CC(═O)Nc12)Nc3c(F)cncc3F
FCOc1ccc2C(═CC(═O)Nc2c1OCCCCCCN3CCC(F)CC3)Nc4c(F)cncc4F
Fc1cncc(F)c1NC2=CC(═O)Nc3c(OCCCCCCn4ncnn4)c(OC(F)(F)F)ccc23
FC(F)Oc1ccc2C(═CC(═O)Nc2c1OCCCCCCOc3ccccn3)Nc4c(F)cncc4F
COCCOCCCCCCOc1c(OC)ccc2C(═CC(═O)Nc12)Nc3c(F)cncc3F
C[N+](C)(C)CCNC(═O)COc1c(OCF)ccc2C(═CC(═O)Nc12)Nc3c(F)cncc3F
COc1ccc2C(═CC(═O)Nc2c1OCC(═O)N(C)CCC[N+](C)(C)C)Nc3c(F)cncc3F
CC(C)C[C@H](NC(═O)COc1c(OC(F)(F)F)ccc2C(═CC(═O)Nc12)Nc3c(F)cncc3F)C(═O)O
C(═O)[C@H](Cc1c[nH]c2ccccc12)NC(═O)COc3c(OCF)ccc4C(═CC(═O)Nc34)Nc5c(F)cncc5F
OC(═O)[C@H](Cc1cnc[nH]1)NC(═O)COc2c(OC(F)(F)F)ccc3C(═CC(═O)Nc23)Nc4c (F)cncc4F
NC(═O)CC[C@H](NC(═O)COc1c(OC(F)F)ccc2C(═CC(═O)Nc12)Nc3c(F)cncc3F)C(═O)O
CC(C)[C@H](NC(═O)CCCOc1c(OC(F)F)ccc2C(═CC(═O)Nc12)Nc3c(F)cncc3F)C(═O)O
COc1ccc2C(═CC(═O)Nc2c1OCCCC(═O)N3CCC[C@H]3C(═O)O)Nc4c(F)cncc4F
OC(═O)[C@H](Cc1c[nH]c2ccccc12)NC(═O)CCCOc3c(OCF)ccc4C(═CC(═O)Nc34)N c5c(F)cncc5F
OC(═O)[C@H](Cc1cnc[nH]1)NC(═O)CCCOc2c(OC(F)(F)F)ccc3C(═CC(═O)Nc23)N c4c(F)cncc4F
NC(═O)CC[C@H](NC(═O)CCCOc1c(OCF)ccc2C(═CC(═O)Nc12)Nc3c(F)cncc3F)C(═O)O
COc1ccc2C(═CC(═O)Nc2c1OCCCCC(═O)NCCN(C)C)Nc3c(F)cncc3F
CC[C@H](C)[C@H](NC(═O)CCCCOc1c(OC(F)F)ccc2C(═CC(═O)Nc12)Nc3c(F)cnc c3F)C(═O)O
CSCC[C@H](NC(═O)CCCCOc1c(OCF)ccc2C(═CC(═O)Nc12)Nc3c(F)cncc3F)C(═O)
CSCC[C@H](NC(═O)CCCCOc1c(OC(F)(F)F)ccc2C(═CC(═O)Nc12)Nc3c(F)cncc3F) C(═O)O
NC(═O)CCCCCOc1c(OC(F)F)ccc2C(═CC(═O)Nc12)Nc3c(F)cncc3F
CN(C)CCN(C)C(═O)CCCCCOc1c(OCF)ccc2C(═CC(═O)Nc12)Nc3c(F)cncc3F
COc1ccc2C(═CC(═O)Nc2c1OCCCCCC(═O)N(C)CCCN(C)C)Nc3c(F)cncc3F
NCCCC[C@H](NC(═O)CCCCCOc1c(OC(F)(F)F)ccc2C(═CC(═O)Nc12)Nc3c(F)cncc 3F)C(═O)O
CCCCN(C)CCCCOc1c(OCF)ccc2C(═CC(═O)Nc12)Nc3c(F)cncc3Cl
CCCCCN(C)CCCCOc1c(OCF)ccc2C(═CC(═O)Nc12)Nc3c(F)cncc3Cl
FC(F)Oc1ccc2C(═CC(═O)Nc2c1OCCCCn3cnnn3)Nc4c(F)cncc4Cl
COCCOCCCCOc1c(OC)ccc2C(═CC(═O)Nc12)Nc3c(F)cncc3Cl
OCCOCCOCCCCOc1c(OC(F)(F)F)ccc2C(═CC(═O)Nc12)Nc3c(F)cncc3Cl
COc1ccc2C(═CC(═O)Nc2c1OCCCCCCN(C)C)Nc3c(F)cncc3Cl
CN(C)CCCCCOc1c(OC(F)(F)F)ccc2C(═CC(═O)Nc12)Nc3c(F)cncc3Cl
CCN(C)CCCCCOc1c(OCF)ccc2C(═CC(═O)Nc12)Nc3c(F)cncc3Cl
CCN(C)CCCCCOc1c(OC(F)F)ccc2C(═CC(═O)Nc12)Nc3c(F)cncc3Cl
NC(═N)NCCCCCCOc1c(OC(F)F)ccc2C(═CC(═O)Nc12)Nc3c(F)cncc3Cl
COc1ccc2C(═CC(═O)Nc2c1OCCCCCCn3cccc3)Nc4c(F)cncc4Cl
Fc1cncc(Cl)c1NC2=CC(═O)Nc3c(OCCCCCCOc4cccnc4)c(OC(F)(F)F)ccc23
COCCOCCCCCCOc1c(OCF)ccc2C(═CC(═O)Nc12)Nc3c(F)cncc3Cl
C[C@H](NC(═O)COc1c(OCF)ccc2C(═CC(═O)Nc12)Nc3c(F)cncc3Cl)C(═O)O
COc1ccc2C(═CC(═O)Nc2c1OCC(═O)N[C@@H](CC(C)C)C(═O)O)Nc3c(F)cncc3Cl
NCCCC[C@H](NC(═O)COc1c(OC(F)F)ccc2C(═CC(═O)Nc12)Nc3c(F)cncc3Cl)C(═O)O
NC(═N)NCCC[C@H](NC(═O)COc1c(OC(F)F)ccc2C(═CC(═O)Nc12)Nc3c(F)cncc3C 1)C(═O)O
NC(═N)NCCC[C@H](NC(═O)COc1c(OC(F)(F)F)ccc2C(═CC(═O)Nc12)Nc3c(F)cncc 3Cl)C(═O)O
CN(C)CCN(C)C(═O)CCCOc1c(OC(F)(F)F)ccc2C(═CC(═O)Nc12)Nc3c(F)cncc3Cl
COc1ccc2C(═CC(═O)Nc2c1OCCCC(═O)N[C@@H](C)C(═O)O)Nc3c(F)cncc3C1
C(═O)[C@@H]1CCCN1C(═O)CCCOc2c(OCF)ccc3C(═CC(═O)Nc23)Nc4c(F)cncc 4Cl
NC(═O)C[C@H](NC(═O)CCCOc1c(OC(F)F)ccc2C(═CC(═O)Nc12)Nc3c(F)cncc3Cl) C(═O)O
OC(═O)[C@@H]1CCCN1C(═O)CCCCOc2c(OC(F)(F)F)ccc3C(═CC(═O)Nc23)Nc4c(F)cncc4Cl
OC(═O)[C@H](Cc1ccccc1)NC(═O)CCCCOc2c(OC(F)F)ccc3C(═CC(═O)Nc23)Nc4c(F)cncc4Cl
OC(═O)[C@H](Cc1cnc[nH]1)NC(═O)CCCCOc2c(OCF)ccc3C(═CC(═O)Nc23)Nc4c(F)cncc4Cl
COc1ccc2C(═CC(═O)Nc2c1OCCCCC(═O)N[C@@H](CC(═O)N)C(═O)O)Nc3c(F)cn cc3Cl
NC(═O)CCCCCOc1c(OCF)ccc2C(═CC(═O)Nc12)Nc3c(F)cncc3Cl
COc1ccc2C(═CC(═O)Nc2c1OCCCCCC(═O)N[C@@H](CC(C)C)C(═O)O)Nc3c(F)cn cc3Cl
OC(═O)[C@H](Cc1c[nH]c2ccccc12)NC(═O)CCCCCOc3c(OC(F)(F)F)ccc4C(═CC(═O)Nc34)Nc5c(F)cncc5Cl
OC(═O)C[C@H](NC(═O)CCCCCOc1c(OC(F)F)ccc2C(═CC(═O)Nc12)Nc3c(F)cncc3Cl)C(═O)O
Fc1cncc(Br)c1NC2=CC(═O)Nc3c(OCCCCN4CCCCC4)c(OC(F)(F)F)ccc23
COc1ccc2C(═CC(═O)Nc2c1OCCCCn3nccn3)Nc4c(F)cncc4Br
COCCOCCOCCCCOc1c(OC(F)F)ccc2C(═CC(═O)Nc12)Nc3c(F)cncc3Br
OCCOCCOCCOCCCCOc1c(OCF)ccc2C(═CC(═O)Nc12)Nc3c(F)cncc3Br
COc1ccc2C(═CC(═O)Nc2c1OCCCCCN(C)C)Nc3c(F)cncc3Br
Fc1cncc(Br)c1NC2=CC(═O)Nc3c(OCCCCCN4CCCC4)c(OC(F)(F)F)ccc23
FCOc1ccc2C(═CC(═O)Nc2c1OCCCCCN3CCC(F)(F)C3)Nc4c(F)cncc4Br
FC(F)Oc1ccc2C(═CC(═O)Nc2c1OCCCCCOc3ccncc3)Nc4c(F)cncc4Br
OCCOCCOCCCCCOc1c(OCF)ccc2C(═CC(═O)Nc12)Nc3c(F)cncc3Br
NCCCCCCOc1c(OC(F)F)ccc2C(═CC(═O)Nc12)Nc3c(F)cncc3Br
COc1ccc2C(═CC(═O)Nc2c1OCCCCCCNC(═N)N)Nc3c(F)cncc3Br
Fc1cncc(Br)c1NC2=CC(═O)Nc3c(OCCCCCCN4CCCCC4)c(OC(F)(F)F)ccc23
FCOc1ccc2C(═CC(═O)Nc2c1OCCCCCCOc3ccncc3)Nc4c(F)cncc4Br
COc1ccc2C(═CC(═O)Nc2c1OCC(═O)NCC[N+](C)(C)C)Nc3c(F)cncc3Br
CC(C)C[C@H](NC(═O)COc1c(OCF)ccc2C(═CC(═O)Nc12)Nc3c(F)cncc3Br)C(═O)O
CC[C@H](C)[C@H](NC(═O)COc1c(OC(F)(F)F)ccc2C(═CC(═O)Nc12)Nc3c(F)cncc3Br)C(═O)O
OC(═O)[C@H](Cc1ccccc1)NC(═O)COc2c(OC(F)F)ccc3C(═CC(═O)Nc23)Nc4c(F)cn cc4Br
NCCCC[C@H](NC(═O)COc1c(OCF)ccc2C(═CC(═O)Nc12)Nc3c(F)cncc3Br)C(═O)O
CN(CC[N+](C)(C)C)C(═O)CCCOc1c(OCF)ccc2C(═CC(═O)Nc12)Nc3c(F)cncc3Br
CC(C)[C@H](NC(═O)CCCOc1c(OC(F)F)ccc2C(═CC(═O)Nc12)Nc3c(F)cncc3Br)C(═O)O
COc1ccc2C(═CC(═O)Nc2c1OCCCCC(═O)N[C@@H](CCCNC(═N)N)C(═O)O)Nc3c(F)cncc3Br
NC(═O)C[C@H](NC(═O)CCCOc1c(OC(F)(F)F)ccc2C(═CC(═O)Nc12)Nc3c(F)cncc3Br)C(═O)O
COc1ccc2C(═CC(═O)Nc2c1OCCCCC(═O)N(C)CCC[N+](C)(C)C)Nc3c(F)cncc3Br
C[C@H](NC(═O)CCCCOc1c(OCF)ccc2C(═CC(═O)Nc12)Nc3c(F)cncc3Br)C(═O)O
C[C@H](NC(═O)CCCCOc1c(OC(F)F)ccc2C(═CC(═O)Nc12)Nc3c(F)cncc3Br)C(═O) O
CC(C)C[C@H](NC(═O)CCCCOc1c(OC(F)(F)F)ccc2C(═CC(═O)Nc12)Nc3c(F)cncc3Br)C(═O)O
C[N+](C)(C)CCOC(═O)CCCCCOc1c(OCF)ccc2C(═CC(═O)Nc12)Nc3c(F)cncc3Br
CN(C)CCNC(═O)CCCCCOc1c(OC(F)F)ccc2C(═CC(═O)Nc12)Nc3c(F)cncc3Br
COc1ccc2C(═CC(═O)Nc2c1OCCCCCC(═O)NCC[N+](C)(C)C)Nc3c(F)cncc3Br
C[N+](C)(C)CCNC(═O)CCCCCOc1c(OC(F)(F)F)ccc2C(═CC(═O)Nc12)Nc3c(F)cncc 3Br
CN(C)CCCCOc1c(OC(F)F)ccc2C(C(═CC(═O)Nc12)Nc3c(Cl)cncc3Cl
COc1ccc2C(═CC(═O)Nc2c1OCCCCOCCO)Nc3c(Cl)cncc3Cl
OCCOCCCCOc1c(OCF)ccc2C(═CC(═O)Nc12)Nc3c(Cl)cncc3Cl
OCCOCCCCOc1c(OC(F)(F)F)ccc2C(C(═CC(═O)Nc12)Nc3c(Cl)cncc3Cl
COc1ccc2C(═CC(═O)Nc2c1OCCCCCN)Nc3c(Cl)cncc3Cl
FC(F)(F)Oc1ccc2C(═CC(═O)Nc2c1OCCCCCN3CCCC(F)(F)C3)Nc4c(Cl)cncc4Cl
FC(F)Oc1ccc2C(═CC(═O)Nc2c1OCCCCCN3CCC(F)C3)Nc4c(Cl)cncc4Cl
FCOc1ccc2C(═CC(═O)Nc2c1OCCCCCn3nccn3)Nc4c(Cl)cncc4Cl
COc1ccc2C(═CC(═O)Nc2c1OCCCCCn3cnnc3)Nc4c(Cl)cncc4Cl
FC1CCN(CCCCCCOc2c(OC(F)(F)F)ccc3C(═CC(═O)Nc23)Nc4c(Cl)cncc4Cl)CC1
FC(F)Oc1ccc2C(═CC(═O)Nc2c1OCCCCCCN3CCC(F)(F)CC3)Nc4c(Cl)cncc4Cl
OCCOCCOCCCCCCOc1c(OCF)ccc2C(═CC(═O)Nc12)Nc3c(Cl)cncc3Cl
COc1ccc2C(═CC(═O)Nc2c1OCCCCCCOCCOCCOCCO)Nc3c(Cl)cncc3Cl
CN(C)CCOC(═O)COc1c(OC(F)(F)F)ccc2C(═CC(═OCC(═O)Nc12)Nc3c(Cl)cncc3Cl
C[N+](C)(C)CCNC(═O)COc1c(OC(F)F)ccc2C(═CC(═O)Nc12)Nc3c(Cl)cncc3Cl
FCOc1ccc2C(═CC(═O)Nc2c1OCC(═O)N3CCNCC3)Nc4c(Cl)cncc4Cl
COc1ccc2C(═CC(═O)Nc2c1OCC(═O)N[C@@H](Cc3c[nH]c4ccccc34)C(═O)O)Nc5c (Cl)cncc5C1
C(═O)[C@H](Cc1c[nH]c2ccccc12)NC(═O)COc3c(OC(F)F)ccc4C(═CC(═O)Nc34)N c5c(Cl)cncc5C1
C(═O)[C@H](Cc1c[nH]c2ccccc12)NC(═O)CCCOc3c(OCF)ccc4C(═CC(═O)Nc34)N c5c(Cl)cncc5Cl
OC[C@H](NC(═O)CCCOc1c(OC(F)(F)F)ccc2C(═CC(═O)Nc12)Nc3c(Cl)cncc3Cl)C(═O)O
COc1ccc2C(═CC(═O)Nc2c1OCCCC(═O)N[C@@H]([C@@H](C)O)C(═O)O)Nc3c(C 1)cncc3Cl
OC(═O)C[C@H](NC(═O)CCCOc1c(OC(F)F)ccc2C(═CC(═O)Nc12)Nc3c(Cl)cncc3Cl)C(═O)O
OC(═O)CNC(═O)CCCCOc1c(OC(F)(F)F)ccc2C(═CC(═O)Nc12)Nc3c(Cl)cncc3Cl
NCCCC[C@H](NC(═O)CCCCOc1c(OCF)ccc2C(═CC(═O)Nc12)Nc3c(Cl)cncc3Cl)C(═O)O
C(═O)CC[C@H](NC(═O)CCCCOc1c(OC(F)F)ccc2C(═CC(═O)Nc12)Nc3c(Cl)cncc 3Cl)C(═O)O
COc1ccc2C(═CC(═O)Nc2c1OCCCCC(═O)N[C@@H](CCC(═O)N)C(═O))Nc3c(Cl) cncc3Cl
NC(═O)CCCCCOc1c(OCF)ccc2C(═CC(═O)Nc12)Nc3c(Cl)cncc3Cl
COc1ccc2C(═CC(═O)Nc2c1OCCCCCC(═O)OCCN(C)C)Nc3c(Cl)cncc3Cl
C[C@@H](O)[C@H](NC(═O)CCCCCOc1c(OC(F)F)ccc2C(═CC(═O)Nc12)Nc3c(Cl) cncc3Cl)C(═O)O
NC(═O)C[C@H](NC(═O)CCCCCOc1c(OC(F)(F)F)ccc2C(═CC(═O)Nc12)Nc3c(Cl)cn cc3Cl)C(═O)O
CCCCN(C)CCCCOc1c(OC(F)F)ccc2C(═CC(═O)Nc12)Nc3c(Cl)cncc3Br
FC1CCN(CCCCOc2c(OC(F)(F)F)ccc3C(═CC(═O)Nc23)Nc4c(Cl)cncc4Br)C1
COc1ccc2C(═CC(═O)Nc2c1OCCCCn3cccc3)Nc4c(Cl)cncc4Br
FCOc1ccc2C(═CC(═O)Nc2c1OCCCCn3ccnc3)Nc4c(Cl)cncc4Br
FC1CCN(CCCCCOc2c(OC(F)(F)F)ccc3C(═CC(═O)Nc23)Nc4c(Cl)cncc4Br)C1
COc1ccc2C(═CC(═O)Nc2c1OCCCCCn3cnnc3)Nc4c(Cl)cncc4Br
FCOc1ccc2C(═CC(═O)Nc2c1OCCCCCn3ncnn3)Nc4c(Cl)cncc4Br
COCCOCCOCCCCCOc1c(OC(F)F)ccc2C(═CC(═O)Nc12)Nc3c(Cl)cncc3Br
NCCCCCCOc1c(OCF)ccc2C(═CC(═O)Nc12)Nc3c(Cl)cncc3Br
COCCN(C)CCCCCCOc1c(OC(F)(F)F)ccc2C(═CC(═O)Nc12)Nc3c(Cl)cncc3Br
FC(F)Oc1ccc2C(═CC(═O)Nc2c1OCCCCCCN3CCCCC3)Nc4c(Cl)cncc4Br
COCCOCCCCCCOc1c(OC)ccc2C(═CC(═O)Nc12)Nc3c(Cl)cncc3Br
NC(═O)COc1c(OC(F)(F)F)ccc2C(═CC(═O)Nc12)Nc3c(Cl)cncc3Br
COc1ccc2C(═CC(═O)Nc2c1OCC(═O)OCC[N+](C)(C)C)Nc3c(Cl)cncc3Br
CC(C)[C@H](NC(═O)COc1c(OCF)ccc2C(═CC(═O)Nc12)Nc3c(Cl)cncc3Br)C(═O)O
OC(═O)[C@H](Cc1ccc(O)cc1)NC(═O)COc2c(OC(F)F)ccc3C(═CC(═O)Nc23)Nc4c(C 1)cncc4Br
COc1ccc2C(═CC(═O)Nc2c1OCCCC(═O)N[C@@H](C)C(═O)O)Nc3c(Cl)cncc3Br
OC(═O)[C@H](Cc1ccc(O)cc1)NC(═O)CCCOc2c(OC(F)(F)F)ccc3C(═CC(═O)Nc23) Nc4c(Cl)cncc4Br
OC(═O)C[C@H](NC(═O)CCCOc1c(OCF)ccc2C(═CC(═O)Nc12)Nc3c(Cl)cncc3Br)C(═O)O
C(═O)CC[C@H](NC(═O)CCCOc1c(OC(F)F)ccc2C(═CC(═O)Nc12)Nc3c(Cl)cncc3Br)C(═O)O
COc1ccc2C(═CC(═O)Nc2c1OCCCCC(═O)OCC[N+](C)(C)C)Nc3c(Cl)cncc3Br
CN(C)CCCN(C)C(═O)CCCCOc1c(OCF)ccc2C(═CC(═O)Nc12)Nc3c(Cl)cncc3Br
CSCC[C@H](NC(═O)CCCCOc1c(OC(F)F)ccc2C(═CC(═O)Nc12)Nc3c(Cl)cncc3Br)C (═O)O
OC[C@H](NC(═O)CCCCOc1c(OC(F)(F)F)ccc2C(═CC(═O)Nc12)Nc3c(Cl)cncc3Br) C(═O)O
COc1ccc2C(═CC(═O)Nc2c1OCCCCCC(═O)N[C@@H](Cc3ccc(O)cc3)C(═O)O)Nc4c (Cl)cncc4Br
CSCC[C@H](NC(═O)CCCCCOc1c(OCF)ccc2C(═CC(═O)Nc12)Nc3c(Cl)cncc3Br)C(═O)O
C(═O)C[C@H](NC(═O)CCCCCOc1c(OC(F)(F)F)ccc2C(═CC(═O)Nc12)Nc3c(Cl)cn cc3Br)C(═O)O
NC(═O)CC[C@H](NC(═O)CCCCCOc1c(OC(F)F)ccc2C(═CC(═O)Nc12)Nc3c(Cl)cnc c3Br)C(═O)O
NC(═N)NCCCCOc1c(OC(F)(F)F)ccc2C(═CC(═O)Nc12)Nc3c(Br)cncc3Br
FC(F)Oc1ccc2C(═CC(═O)Nc2c1OCCCCN3CCC(F)CC3)Nc4c(Br)cncc4Br
FCOc1ccc2C(═CC(═O)Nc2c1OCCCCn3ccnn3)Nc4c(Br)cncc4Br
COCCOCCOCCCCOc1c(OC)ccc2C(═CC(═O)Nc12)Nc3c(Br)cncc3Br
CCCNCCCCCOc1c(OC)ccc2C(═CC(═O)Nc12)Nc3c(Br)cncc3Br
CCCCN(C)CCCCCOc1c(OC(F)F)ccc2C(═CC(═O)Nc12)Nc3c(Br)cncc3Br
FC1CCN(CCCCCOc2c(OC(F)(F)F)ccc3C(═CC(═O)Nc23)Nc4c(Br)cncc4Br)C1
FCOc1ccc2C(═CC(═O)Nc2c1OCCCCCn3ccnn3)Nc4c(Br)cncc4Br
CCCCN(C)CCCCCCOc1c(OC)ccc2C(═CC(═O)Nc12)Nc3c(Br)cncc3Br
FC(F)Oc1ccc2C(═CC(═O)Nc2c1OCCCCCCN3CCCCC3)Nc4c(Br)cncc4Br
FC(F)(F)Oc1ccc2C(═CC(═O)Nc2c1OCCCCCCn3cccc3)Nc4c(Br)cncc4Br
FCOc1ccc2C(═CC(═O)Nc2c1OCCCCCCn3ncnn3)Nc4c(Br)cncc4Br
OC(═O)CNC(═O)COc1c(OCF)ccc2C(═CC(═O)Nc12)Nc3c(Br)cncc3Br
COc1ccc2C(═CC(═O)Nc2c1OCC(═O)N[C@@H](C)C(═O)O)Nc3c(Br)cncc3Br
OC(═O)[C@H](Cc1ccccc1)NC(═O)COc2c(OC(F)(F)F)ccc3C(═CC(═O)Nc23)Nc4c(B r)cncc4Br
OC(═O)[C@H](Cc1ccc(O)cc1)NC(═O)COc2c(OC(F)F)ccc3C(═CC(═O)Nc23)Nc4c(B r)cncc4Br
COc1ccc2C(═CC(═O)Nc2c1OCCCC(═O)NCCN(C)C)Nc3c(Br)cncc3Br
CN(C)CCCN(C)C(═O)CCCOc1c(OC(F)F)ccc2C(═CC(═O)Nc12)Nc3c(Br)cncc3Br
OC(═O)[C@H](Cc1ccc(O)cc1)NC(═O)CCCOc2c(OC(F)(F)F)ccc3C(═CC(═O)Nc23) Nc4c(Br)cncc4Br
OC[C@H](NC(═O)CCCOc1c(OCF)ccc2C(═CC(═O)Nc12)Nc3c(Br)cncc3Br)C(═O)O
OC[C@H](NC(═O)CCCOc1c(OC(F)(F)F)ccc2C(═CC(═O)Nc12)Nc3c(Br)cncc3Br)C(═O)O
COc1ccc2C(═CC(═O)Nc2c1OCCCCC(═O)N(C)CC[N+](C)(C)C)Nc3c(Br)cncc3Br
CC(C)C[C@H](NC(═O)CCCCOc1c(OC(F)(F)F)ccc2C(═CC(═O)Nc12)Nc3c(Br)cncc 3Br)C(═O)O
OC(═O)[C@@H]1CCCN1C(═O)CCCCOc2c(OCF)ccc3C(═CC(═O)Nc23)Nc4c(Br)cn cc4Br
C[C@@H](O)[C@H](NC(═O)CCCCOc1c(OC(F)F)ccc2C(═CC(═O)Nc12)Nc3c(Br)c ncc3Br)C(═O)O
CN(C)CCN(C)C(═O)CCCCCOc1c(OCF)ccc2C(═CC(═O)Nc12)Nc3c(Br)cncc3Br
C[C@H](NC(═O)CCCCCOc1c(OC(F)(F)F)ccc2C(═CC(═O)Nc12)Nc3c(Br)cncc3Br) C(═O)O
CC(C)[C@H](NC(═O)CCCCCOc1c(OC(F)F)ccc2C(═CC(═O)Nc12)Nc3c(Br)cncc3Br)C(═O)O
COc1ccc2C(═CC(═O)Nc2c1OCCCCCC(═O)N[C@@H](Cc3ccccc3)C(═O)O)Nc4c(Br)cncc4Br
NC(═O)CC[C@H](NC(═O)CCCCCOc1c(OC(F)(F)F)ccc2C(═CC(═O)Nc12)Nc3c(Br) cncc3Br)C(═O)O
COC1=CC═C(C(NC2=C(C)C═NC═C2C)═CC(N3)=O)C3=C1CCCCCCN(CCO)
COC1=CC═C(C(NC2=C(C)C═NC═C2C)═CC(N3)=O)C3=C1CCCCCCN(CCO)CC O
COC1=CC═C(C(NC2=C(C)C═NC═C2C)═CC(N3)=O)C3=C1CCCCCCNCCCO
COC1=CC═C(C(NC2=C(C)C═NC═C2C)═CC(N3)=O)C3=C1CCCCCCN4CCNCC4
COC1=CC═C(C(NC2=C(C)C═NC═C2C)═CC(N3)=O)C3=C1OCCCCCCN4CCCNC C4
COC1=CC═C(C(NC2=C(C)C═NC═C2C)═CC(N3)=O)C3=C1CCCCCCN4CCCN(C)CC4
COC1=CC═C(C(NC2=C(C)C═NC═C2C)═CC(N3)=O)C3=C1CCCCCCNCCCN(C) C
COC1=CC═C(C(NC2=C(C)C═NC═C2Cl)═CC(N3)=O)C3=C1OCCCCCCN4CCOCC 4
COC1=CC═C(C(NC2=C(C)C═NC═C2Cl)═CC(N3)=O)C3=C1OCCCCCCN4CCCCC 4
COC1=CC═C(C(NC2=C(C)C═NC═C2Cl)═CC(N3)=O)C3=C1OCCCCCCN4CCCC4
COC1=CC═C(C(NC2=C(C)C═NC═C2Cl)═CC(N3)=O)C3=C1OCCCCCCN(C)C
COC1=CC═C(C(NC2=C(C)C═NC═C2Cl)═CC(N3)=O)C3=C1OCCCCCCNCCO
COC1=CC═C(C(NC2=C(C)C═NC═C2Cl)═CC(N3)=O)C3=C1OCCCCCCN(C)CCO
COC1=CC═C(C(NC2=C(C)C═NC═C2Cl)═CC(N3)=O)C3=C1OCCCCCCN(CCO)CC O
COC1=CC═C(C(NC2=C(C)C═NC═C2Cl)═CC(N3)=O)C3=C1OCCCCCCNCCCO
COC1=CC═C(C(NC2=C(C)C═NC═C2Cl)═CC(N3)=)C3=C1CCCCCCN(C)CCCO
COC1=CC═C(C(NC2=C(C)C═NC═C2Cl)═CC(N3)=O)C3=C1OCCCCCCN4CCCNC C4
COC1=CC═C(C(NC2=C(C)C═NC═C2Cl)═CC(N3)=O)C3=C1OCCCCCCN4CCCN(C)CC4
COC1=CC═C(C(NC2=C(C)C═NC═C2Cl)═CC(N3)=O)C3=C1OCCCCCCNCCCN(C) C
COC1=CC═C(C(NC2=C(C)C═NC═C2Cl)═CC(N3)=O)C3=C1OCCCCCCN4CCN(C) CC4

The activity of the compounds in Examples 1-98 PDE4 inhibitors is illustrated in the following assay. The other compounds listed above, which have not yet been made and/or tested, are predicted to have activity in this assay as well.

### PDE/AK Biological Activity Assay Protocol

Phosphodiesterases regulate the activity of the second messenger cyclic adenosine monophosphate (cAMP) by hydrolyzing cAMP to adenosine monophosphate (AMP). We developed a coupled PDE/AK bioluminescent high-throughput assay to measure the activity of phosphodiesterases. In the PDE/AK assay, the AMP produced from the hydrolysis of cAMP by phosphodiesterase is converted to adenosine diphosphate (ADP) by the phosphotransferase activity of adenylate kinase (also known as ADK or myokinase). Adenylate kinase will catalyze the conversion of 1 mole of AMP and 1 mole of ATP into 2 moles of ADP. Thus the conversion of AMP to ADP will result in the subsequent loss of ATP. The decrease in ATP concentration can be monitored as light output using luciferase in a reaction that requires ATP to produce light. The PDE/AK assay uses luciferase, which catalyzes the production of light from ATP present in the reaction mix. The amount of emitted light is inversely proportional to the level of AMP present in the reaction. Test compounds are incubated with the phosphodiesterases and phosphodiesterase inhibitors will decrease the rate of cAMP hydrolysis and decrease the subsequent production of AMP. Thus phosphodiesterase inhibitors will decrease the conversion of ATP to ADP and result in the greatest emission of light.

Compounds may be screened for functional potency in in vitro biochemical assays for their ability to inhibit the activity of phosphodiesterase (PDE) enzymes. A coupled PDE/AK assay was utilized to measure the inhibitory activity of compounds on PDE isoforms. The catalytic domain of PDE4B was fused to an amino-terminus hex-histidine affinity tag and purified. PDE4B enzyme was incubated with compounds for 10 minutes. Cyclic AMP substrate, ATP and adenylate kinase were then added to the PDE enzyme mix and incubated for 45 minutes at room temperature. A commercial ATP detection assay, Perkin Elmer's easyLite ATP detection reagent, was then added to measure the relative amount of ATP present in the reaction. After 10 minute incubation, the luminescent signal was quantitated with either the Perkin Elmer Viewlux or Molecular Devices Acquest or Analyst. Roflumilast and Rolipram are the positive controls used in the assay.

The PDE/AK assay can be performed with any cAMP dependent phosphodiesterase, and can be performed in 96-, 384-, or 1536-well plates. Unlike other phosphodiesterase activity assays, the PDE/AK assay does not require radioactive substrates, modified substrates, beads, or antibodies, which each have disadvantages. Specifically, the use of radioactive substrates in a phosphodiesterase activity assay requires special protocols for handling and waste disposal, and reduced throughput. Modified substrates add the additional complexity of potentially altering the binding to the phosphodiesterase catalytic site. Fluorescently modified substrates may cause fluorescence artifacts that may interfere in the interpretation of assay results. The use of beads that selectively bind to either the cAMP or AMP may cause an additional complexity in reagent handling. The use of antibodies that selectively bind to either cAMP or AMP can be extremely costly with a relatively small signal to noise ratio. Therefore, the PDE/AK assay represents a simple, cost-effective alternative to known methods.

Additionally, the PDE/AK assay's mechanism offers advantages which make it useful for comparison to other phosphodiesterase activity assays. In conventional assays in which cAMP concentration is inversely proportional to the output of bioluminescence (such as Cambrex Biosciences's PDELight), phosphodiesterase catalyzes the hydrolysis of cAMP to AMP that is subsequently converted to ATP, consumed by a luciferase enzyme, and results in an increased bioluminescent output. A phosphodiesterase inhibitor prevents the hydrolysis of cAMP to AMP and consequently results in a decreased bioluminescent output. However, compounds which exhibit luciferase inhibition activity would score as a false positive for PDE inhibition in the conventional assay. In contrast, the PDK/AK assay would not score luciferase inhibitors as false positive for PDE inhibition because inhibition of PDE activity would result in increased bioluminescence. However, compounds that are capable of inhibiting both PDE and luciferase would be false negatives. Compounds that inhibit adenylate kinase would score as false positives in the PDE/AK assay. Use of both types of assay, and comparison of the data from both, would yield a richer and more reliable data set.

Accordingly, contemplated herein is a method for the detection and quantitation of PDE inhibition which uses adenylate kinase coupled to an ATP-depleting assay.

In certain embodiments, said method comprises the steps of:
1. A first incubation, of PDE, or an isolated isoform thereof, with a compound to be tested;
2. Addition of ATP, cAMP, and AK substrates;
3. A second incubation of said PDE;
4. Coupling to a means for readout of ATP-depletion; and
5. Readout.

In further embodiments, said first incubation is for between about 5 and about 15 minutes; in yet further embodiments, said first incubation is for about 10 minutes.

In further embodiments, said second incubation is for longer than about 30 minutes; in yet further embodiments, said sufficient length of time is between about 30 minutes and about one hour; in even further embodiments, sufficient length of time is about 45 minutes.

In certain embodiments, said readout means is a luciferase assay. In further embodiments, said luciferase assay is a Perkin Elmer easyLite assay.

In further embodiments, said readout is done on a machine for detecting a luminescent signal. In other embodiments, said machine is selected from the group consisting of a Perkin Elmer Viewlux, a Molecular Devices Acquest, and a Molecular Devices Analyst.

Results are shown below in Table 1.

Data reported as NT refers to the example having been not tested. It is expected that these compounds when tested will be active and will have utility similar to those that have been tested.

TABLE 1
Biological Activity

| | PDE4 Enzyme IC50, |
|---|---|
| | + indicates <1 μM, |
| Example | − indicates >1 μM |
| 1 | + |
| 2 | NT |
| 3 | NT |
| 4 | NT |
| 5 | + |
| 6 | NT |
| 7 | NT |
| 8 | + |
| 9 | NT |
| 10 | NT |
| 11 | + |
| 12 | + |
| 13 | + |
| 14 | + |
| 15 | + |
| 16 | + |
| 17 | NT |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 35 | + |
| 36 | + |
| 37 | + |
| 38 | + |
| 39 | NT |
| 40 | NT |
| 41 | + |
| 42 | + |
| 43 | NT |
| 44 | + |
| 45 | + |
| 46 | NT |
| 47 | + |
| 48 | NT |
| 49 | + |
| 50 | + |
| 51 | + |
| 52 | + |
| 53 | + |
| 54 | + |
| 55 | + |
| 56 | + |
| 57 | + |
| 58 | + |
| 59 | NT |
| 60 | NT |
| 61 | + |
| 62 | + |
| 63 | + |
| 64 | + |
| 65 | NT |
| 66 | + |
| 67 | + |
| 68 | + |
| 69 | + |
| 70 | + |
| 71 | + |
| 72 | + |
| 73 | + |
| 74 | + |
| 75 | + |
| 76 | + |
| 77 | + |
| 78 | + |
| 79 | + |
| 80 | + |
| 81 | + |
| 82 | + |
| 83 | + |
| 84 | + |
| 85 | + |
| 86 | + |
| 87 | + |
| 88 | + |
| 89 | NT |
| 90 | NT |
| 91 | NT |
| 92 | NT |
| 93 | NT |
| 94 | + |
| 95 | + |
| 96 | + |
| 97 | + |
| 98 | + |

### In Vivo Assay Number One

### Lacrimal Gland Inflammation-Induced Dry Eye in Rabbits

A more detailed description of the assay used herein may be found in Naglehout, T J et al. 2005, “Preservation of Tear Film Integrity and Inhibition of Corneal Injury by Dexamethasone in a Rabbit Model of Lacrimal Gland Inflammation-Induced Dry Eye,” J. Ocular Pharm. Ther., 21:2. 139-148, the disclosure of which is hereby incorporated by reference as if written herein in its entirety.

### Initiation of Dry Eye and Treatment

Rabbits (n=4/group) were randomized into treatment groups and dosed topically BID with maxidex vehicle or test drug beginning one day prior to initiation of dry eye. Each rabbit received bilateral lacrimal gland injections of saline or ConA (300 μg/30 μl) and topical ocular dosing was continued BID throughout the study.

### Corneal Staining

Desiccation was initiated 1-2 hours following lacrimal gland injection by placing conscious rabbits in the environmental chamber (10-15% humidity/72° F.). Following 72 consecutive hours of exposure to this dry environment, the animals were anesthetized and sutures were placed in each upper and lower eyelid and lifted to form a corneal/conjunctival cup. Methylene blue dye (1 mL, 1% in dH₂O) was added to the cup for three minutes and the excess removed by washing with 200 mL BSS®. The contralateral eye was then stained using the same procedure. Rabbits were euthanized immediately following the staining procedure and the eyes were excised. The corneas were isolated with a trephine and the 9.5-mm punch of the cornea was placed in 2 mL acetone/saturated sodium sulfate (7:3, vol./vol.). Absorbance of the extracted dye was determined at 660 nm.

### Tear Breakup Time (TBUT)

TBUT was quantified in a separate group of treated animals. TBUT was determined by instilling 5 μL sodium fluorescein into the cul de sac and manually blinking the lids to distribute the fluorescein within the tear film. Under slit lamp observation, the eye was held open and the time whereby one or more black spots or streaks appeared in the precorneal tear film was recorded. The rabbits were euthanized 3 days following ConA injection.

Data reported as NT refers to the example having been not tested.

TABLE 2
In Vivo Activity

| Example | Activity at Percent Concentration Tested | | | | | | |
|---|---|---|---|---|---|---|---|
| No. | 1 × 10⁻⁶ | 1 × 10⁻⁵ | 1 × 10⁻⁴ | 1 × 10⁻³ | 1 × 10⁻² | 1 × 10⁻¹ | 1 |
| 1 | NT | NT | NT | NT | Inactive | Inactive | Inactive |
| 5 | Inactive | NT | Inactive | NT | Inactive | NT | NT |
| 8 | Inactive | NT | Inactive | NT | Inactive | NT | NT |

### In Vivo Assay Number Two

### Guinea Pig Passive Conjunctival Anaphylaxis

Male Hartley VAF outbred guinea pigs were passively sensitized to ovalbumin by a single OD subconjunctival injection of undiluted guinea pig anti-ovalbumin antiserum 24 hours before OD topical challenge with 500 μg ovalbumin in saline. Control animals were injected with saline only and challenged with ovalbumin. To determine acute phase drug efficacy, 30 min after challenge animals were clinically scored by a masked observer for severity of signs of conjunctivitis based on a standard scale. Test compounds were administered as a 20 μL topical drop 1 hour prior to challenge (QD), or 1 hour prior to challenge and again 8 hours after challenge (BID). Positive control compound was dexamethasone (Dex) at 0.1% w/v, formulated similarly. Twenty-four hours after challenge, animals were euthanized and conjunctivae were harvested for determination of tissue eosinophil peroxidase (EPO) concentration as a marker of allergic inflammation. Homogenates of freshly collected tissues were prepared by shaking the tissues in 2 mL round-bottom tubes containing 0.5 mL of homogenization buffer (50 mM Tris HCl, pH 8.0, 6 mM KBr) and one 5-mm stainless steel bead on a Qiagen TissueLyser at 30 Hz for 5 min. Homogenates were frozen and thawed once, then centrifuged at 10,000 rpm for 5 min. EPO activity in supernatants was measured by reacting diluted homogenates with a solution of 6 mM o-phenylenediamine substrate and 8.8 mM H₂O₂ in homogenization buffer for 3 min. The reaction was stopped with 4M H₂SO₄ and absorbances were measured at 490 nm on a spectrophotometric plate reader. Total EPO in samples was calculated from a standard curve of recombinant human EPO in each assay. EPO activity was normalized to total protein concentration (Pierce BCA assay) in supernatants. Background EPO activity was determined from the unsensitized, antigen-challenged control group. Percent inhibition was calculated from the sensitized, antigen-challenged vehicle-treated control group in each experiment. Groups were compared by ANOVA with Dunnett's or Tukey's post-hoc tests where appropriate with significance assigned at the 95% confidence level.

Data reported as NT refers to the example having been not tested. It is expected that these compounds when tested will be active and will have utility similar to those that have been tested.

TABLE 3
In Vivo Activity

| | ++: test compound at 0.01% w/v is statistically |
|---|---|
| | equivalent to Dex |
| | +: test compound at 0.1% w/v is statistically |
| | equivalent to Dex |
| | −: test compound at 0.1% w/v is not statistically |
| Example | equivalent to Dex |
| 1 | ++ |
| 2 | NT |
| 3 | NT |
| 4 | NT |
| 5 | ++ |
| 6 | NT |
| 7 | NT |
| 8 | ++ |
| 9 | NT |
| 10 | NT |
| 11 | + |
| 12 | NT |
| 13 | NT |
| 14 | + |
| 15 | ++ |
| 16 | ++ |
| 17 | NT |
| 18 | + |
| 19 | + |
| 20 | ++ |
| 21 | + |
| 22 | + |
| 23 | NT |
| 24 | NT |
| 25 | + |
| 26 | + |
| 27 | ++ |
| 28 | ++ |
| 29 | NT |
| 30 | ++ |
| 31 | ++ |
| 32 | + |
| 33 | ++ |
| 34 | NT |
| 35 | + |
| 36 | NT |
| 37 | + |
| 38 | ++ |
| 39 | NT |
| 40 | NT |
| 41 | NT |
| 42 | NT |
| 43 | NT |
| 44 | NT |
| 45 | + |
| 46 | NT |
| 47 | NT |
| 48 | NT |
| 49 | + |
| 50 | + |
| 51 | ++ |
| 52 | ++ |
| 53 | NT |
| 54 | NT |
| 55 | ++ |
| 56 | NT |
| 57 | NT |
| 58 | NT |
| 59 | NT |
| 60 | NT |
| 61 | NT |
| 62 | NT |
| 63 | NT |
| 64 | NT |
| 65 | NT |
| 66 | NT |
| 67 | NT |
| 68 | NT |
| 69 | NT |
| 70 | NT |
| 71 | NT |
| 72 | ++ |
| 73 | ++ |
| 74 | NT |
| 75 | NT |
| 76 | NT |
| 77 | NT |
| 78 | NT |
| 79 | NT |
| 80 | NT |
| 81 | NT |
| 82 | NT |
| 83 | NT |
| 84 | NT |
| 85 | NT |
| 86 | NT |
| 87 | NT |
| 88 | NT |
| 89 | NT |
| 90 | NT |
| 91 | NT |
| 92 | NT |
| 93 | NT |
| 94 | NT |
| 95 | NT |
| 96 | NT |
| 97 | NT |
| 98 | NT |

### Solubility Protocol

About 5 mg of a compound was mixed with 500 μL of pH 7.4, 0.1 M sodium phosphate buffer. The mixture was adjusted to the original pH of 7.4 and then mixed overnight or longer via rotary mixing. The sample was checked for pH and then filtered through a 0.45 m filter. If the pH drifted away, the sample was adjusted to its original pH and mixed for at least 15 minutes before filtration. The filtrate was analyzed using HPLC.

Data reported as NT refers to the example having been not tested. It is expected that these compounds when tested will be active and will have utility similar to those that have been tested.

TABLE 4
Solubility

| | Solubility, pH 7.4, |
|---|---|
| | + indicates ≧1 mg/mL, |
| Example | − indicates <1 mg/mL |
| 1 | + |
| 2 | NT |
| 3 | NT |
| 4 | NT |
| 5 | + |
| 6 | NT |
| 7 | NT |
| 8 | NT |
| 9 | NT |
| 10 | NT |
| 11 | + |
| 12 | + |
| 13 | NT |
| 14 | + |
| 15 | + |
| 16 | + |
| 17 | NT |
| 18 | + |
| 19 | + |
| 20 | + |
| 21 | + |
| 22 | + |
| 23 | NT |
| 24 | NT |
| 25 | + |
| 26 | + |
| 27 | + |
| 28 | + |
| 29 | + |
| 30 | + |
| 31 | NT |
| 32 | + |
| 33 | + |
| 34 | NT |
| 35 | + |
| 36 | + |
| 37 | + |
| 38 | + |
| 39 | NT |
| 40 | NT |
| 41 | + |
| 42 | + |
| 43 | NT |
| 44 | NT |
| 45 | + |
| 46 | NT |
| 47 | + |
| 48 | NT |
| 49 | + |
| 50 | + |
| 51 | + |
| 52 | + |
| 53 | + |
| 54 | + |
| 55 | + |
| 56 | + |
| 57 | + |
| 58 | + |
| 59 | NT |
| 60 | NT |
| 61 | NT |
| 62 | + |
| 63 | NT |
| 64 | + |
| 65 | NT |
| 66 | + |
| 67 | + |
| 68 | + |
| 69 | NT |
| 70 | + |
| 71 | + |
| 72 | + |
| 73 | + |
| 74 | + |
| 75 | + |
| 76 | − |
| 77 | − |
| 78 | NT |
| 79 | NT |
| 80 | + |
| 81 | NT |
| 82 | NT |
| 83 | NT |
| 84 | NT |
| 85 | NT |
| 86 | NT |
| 87 | NT |
| 88 | NT |
| 89 | NT |
| 90 | NT |
| 91 | NT |
| 92 | NT |
| 93 | NT |
| 94 | NT |
| 95 | NT |
| 96 | NT |
| 97 | NT |
| 98 | NT |

### Stability Protocol

About 3 mg of a compound was dissolved in 3 mL of acetonitrile/water mixture (50/50). 100 μL of the stock solution was added to 20 mL each of 0.025 M pH 5.0 sodium acetate buffer (A5.0), 0.025 M pH 5.0 citrate buffer (C5.0) and 0.025 M pH 7.4 sodium phosphate buffer (P7.4). A two mL aliquot of each solution was kept in a glass vial equipped a cap lined with Teflon. Two vials containing the solution were stored at 4° C. or −20° C. as control. Other aliquots were stressed at RT, 45° C. or 75° C. for specified time check points, e.g. 1 day, 1 w, 2 w, 4 w, 8 w, 12 w. The stressed samples in duplicate were assayed against the control using HPLC. The results were reported in % degradation. Preliminary shelf life (T90) was estimated assuming the rate of chemical degradation is double when the temperature is up 10° C.

Data reported as NT refers to the example having been not tested. It is expected that these compounds when tested will be active and will have utility similar to those that have been tested.

TABLE 5
Stability

| | Stability, pH 7.4 |
|---|---|
| | + indicates ≧1 Year, |
| Example | − indicates <1 Year |
| 1 | + |
| 2 | NT |
| 3 | NT |
| 4 | NT |
| 5 | + |
| 6 | NT |
| 7 | NT |
| 8 | + |
| 9 | NT |
| 10 | NT |
| 11 | + |
| 12 | NT |
| 13 | NT |
| 14 | + |
| 15 | + |
| 16 | + |
| 17 | NT |
| 18 | + |
| 19 | + |
| 20 | + |
| 21 | + |
| 22 | + |
| 23 | NT |
| 24 | NT |
| 25 | NT |
| 26 | NT |
| 27 | NT |
| 28 | NT |
| 29 | NT |
| 30 | NT |
| 31 | NT |
| 32 | NT |
| 33 | NT |
| 34 | NT |
| 35 | + |
| 36 | NT |
| 37 | NT |
| 38 | + |
| 39 | NT |
| 40 | NT |
| 41 | NT |
| 42 | NT |
| 43 | NT |
| 44 | NT |
| 45 | NT |
| 46 | NT |
| 47 | NT |
| 48 | NT |
| 49 | NT |
| 50 | NT |
| 51 | NT |
| 52 | + |
| 53 | + |
| 54 | + |
| 55 | NT |
| 56 | + |
| 57 | + |
| 58 | + |
| 59 | NT |
| 60 | NT |
| 61 | + |
| 62 | NT |
| 63 | NT |
| 64 | NT |
| 65 | NT |
| 66 | NT |
| 67 | NT |
| 68 | NT |
| 69 | + |
| 70 | NT |
| 71 | NT |
| 72 | NT |
| 73 | NT |
| 74 | NT |
| 75 | NT |
| 76 | NT |
| 77 | NT |
| 78 | + |
| 79 | + |
| 80 | NT |
| 81 | NT |
| 82 | NT |
| 83 | NT |
| 84 | NT |
| 85 | NT |
| 86 | NT |
| 87 | NT |
| 88 | NT |
| 89 | NT |
| 90 | NT |
| 91 | NT |
| 92 | NT |
| 93 | NT |
| 94 | NT |
| 95 | NT |
| 96 | NT |
| 97 | NT |
| 98 | NT |

### COMPOSITIONS

The following are examples of compositions which may be used to topically deliver compounds disclosed herein, for example to the eye or nasal passages.

### Composition Example 1

| Ingredients | Concentration (w/v %) |
|---|---|
| Compound of Formula (VI) | 0.1% |
| Dibasic Sodium Phosphate (Anhydrous) | 0.5% |
| Sodium Chloride | 0.65% |
| Benzalkonium chloride | 0.01% |
| Sodium Hydroxide/Hydrochloric Acid | For adjusting pH to 7.0 ± 0.2 |
| Purified Water | q.s. to 100% |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula VI 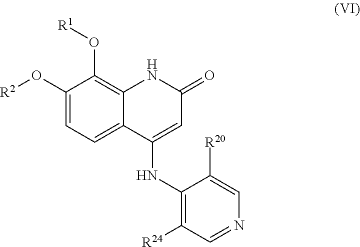
or a salt thereof, wherein:
R¹ and R² are independently selected from the group consisting of —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, heteroaryl, heteroaralkyl, and hydroxyalkyl, any of which may be optionally substituted;
s is 1-8;
G¹ is selected from the group consisting of alkoxy, amino, amido, carbonyl, hydroxy, ether, an amino acid, and null;
G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;
G³ is selected from the group consisting of alkyl, alkoxy, amino, hydroxy, ether, carboxyl, hydroxamic acid, an amino acid, phosphonate, phosphoramide, and null, any of which may be optionally substituted; and
R²⁰ and R²⁴ are independently selected from the group consisting of hydrogen, halogen, hydroxyl, lower alkyl, lower alkoxy, lower haloalkyl, lower haloalkoxy, amino, and carboxyl.

2. The compound as recited in claim 1, wherein R²⁰ and R²⁴ are independently selected from the group consisting of hydrogen, halogen, and lower alkyl.

3. The compound as recited in claim 2, wherein:
R¹ and R² are independently selected from the group consisting of —(CH₂)ₛG¹G²G³, acyl, acylalkyl, C₁-C₁₀ alkyl, aminoalkyl, alkenyl, alkynyl, carboxyalkyl, heteroalkyl, cycloalkyl, cycloalkylalkyl, heterocycloalkyl, heterocycloalkylalkyl, aralkyl, aryl, heteroaryl, and heteroaralkyl, any of which may be optionally substituted; and
G² is selected from the group consisting of alkyl, alkoxy, amino, aryl, halo, heterocycloalkyl, heteroaryl, carboxylalkylamino, an amino acid, and null, any of which may be optionally substituted with a substituent selected from the group consisting of lower alkyl, amino, alkylamino, alkylaminoalkyl, aminoalkylamino, aminoalkyl, hydroxylalkyl, hydroxylalkylamino, cycloalkyl, heterocycloalkyl, and heterocycloalkylalkyl.

4. The compound as recited in claim 3, wherein R²⁰ and R²⁴ are independently selected from the group consisting of halogen and lower alkyl.

5. The compound as recited in claim 4, wherein R² is lower alkyl.

6. The compound as recited in claim 5, wherein:
R¹ is selected from the group consisting of —(CH₂)ₛG¹G²G³, lower alkyl, cycloalkyl, cycloalkylalkyl, and heterocycloalkyl, any of which may be optionally substituted;
s is 1-6;
G¹ is selected from the group consisting of amino, amido, and null;
G² is selected from the group consisting of alkoxy, amino, aryl, halo, heterocycloalkyl, and null; and
G³ is selected from the group consisting of alkyl, carboxyl, and null, any of which may be optionally substituted.

7. The compound as recited in claim 6, wherein R² is methyl.

8. The compound as recited in claim 7, wherein R¹ is —(CH₂)ₛG².

9. The compound as recited in claim 8, wherein s is 6.

10. The compound as recited in claim 9, wherein R²⁰ and R²⁴ are independently selected from the group consisting of halogen and methyl.

11. The compound as recited in claim 10, wherein said halogen is chlorine.

12. The compound as recited in claim 11, wherein G² is selected from the group consisting of amino and heterocycloalkyl.

13. The compound as recited in claim 12, wherein said heterocycloalkyl is selected from the group consisting of a five-, six-, and seven-membered ring containing one or two nitrogens.

14. The compound as recited in claim 13, wherein G² has a formula selected from the group consisting of 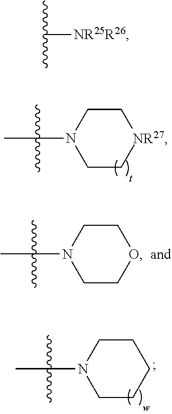
t is 1 or 2;
w is 0 or 1;
R²⁵ and R²⁶ are independently selected from the group consisting of hydrogen, methyl, —(CH₂)ₓOH, and —(CH₂)₃N(CH₃)₂;
x is 2 or 3; and
R²⁷ is selected from the group consisting of hydrogen and methyl.

15. The compound as recited in claim 14, wherein G² has a formula selected from the group consisting of 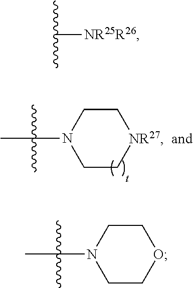
and
R²⁵ and R²⁶ are independently selected from the group consisting of methyl and —(CH₂)ₓOH.

16. The compound as recited in claim 15, wherein R²⁰ and R²⁴ are each chlorine.

17. The compound as recited in claim 16, wherein G² is

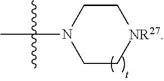

18. The compound as recited in claim 17, wherein R²⁷ is methyl.

19. The compound as recited in claim 18, wherein t is 1.

20. The compound as recited in claim 18, wherein t is 2.

21. The compound as recited in claim 16, wherein G² is

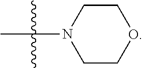 [SMILES: *N1CCOCC1]

22. The compound as recited in claim 16, wherein G² is —NR²⁵R²⁶.

23. The compound as recited in claim 22, wherein:
R²⁵ is methyl; and
R²⁶ is —(CH₂)ₓOH.

24. The compound as recited in claim 23, wherein x is 2.

25. The compound as recited in claim 15, wherein R²⁰ and R²⁴ are each methyl.

26. The compound as recited in claim 25, wherein G² has a formula selected from the group consisting of

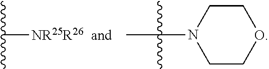

27. The compound as recited in claim 26, wherein G² is —NR²⁵R²⁶.

28. The compound as recited in claim 27, wherein R²⁵ and R²⁶ are each methyl.

29. The compound as recited in claim 26 wherein G² is

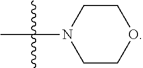 [SMILES: *N1CCOCC1]

30. A compound selected from the group consisting of Examples 1 to 98.

31. A compound as recited in claim 1 for use as a medicament.

32. A compound as recited in claim 1 for use in the manufacture of a medicament for the prevention or treatment of a disease or condition ameliorated by the inhibition of PDE4.

33. The compound as recited in claim 32, wherein said PDE4 is the PDE4B subtype.

34. A pharmaceutical composition comprising a compound as recited in claim 1 together with a pharmaceutically acceptable carrier.

35. A method of inhibition of PDE4 comprising contacting PDE4 with a compound as recited in claim 1.

36. A method of treatment of a PDE4-mediated disease comprising the administration of a therapeutically effective amount of a compound as recited in claim 1 to a patient in need thereof.

37. The method as recited in claim 36 wherein said PDE4-mediated disease is an ophthalmic disease.

38. The method as recited in claim 37, wherein said ophthalmic disease is selected from the group consisting of allergic conjunctivitis, dry eye, glaucoma, corneal neovascularization, optic neuritis, Sjogren's syndrome, retinal ganglion degeneration, ocular ischemia, retinitis, retinopathy, uveitis, ocular photophobia, and inflammation and pain associated with acute injury to the eye tissue.

39. A method of treatment of allergic conjunctivitis to a patient in need thereof comprising the administration of a compound selected from the group consisting of Examples 15, 16, 26, 30, 35, and 38.

40. A method for achieving an effect in a patient comprising the administration of a therapeutically effective amount of a compound as recited in claim 1 to a patient, wherein the effect is selected from the group consisting of treatment of nasal allergies, treatment of ocular allergies, ophthalmic disorders, treatment of dry eye, and analgesia of eye tissue.
